(19) United States
(12) Patent Application Publication
(10) Pub. No.: US 2010/0105680 A1
(43) Pub. Date: Apr. 29, 2010

(54) SUBSTITUTED DIMETHYLCYCLOBUTYL COMPOUNDS, THEIR PREPARATION AND USE IN MEDICAMENTS

(76) Inventors: Maria Rosa Cuberes Altisent, San Cugat del Valles (ES); Jordi Corbera Arjona, Terrasa (ES); Jorg Holenz, Enhoma (SE); Rosa Maria Ortuno Mingarro, Barcelona (ES); Sandra Izquierdo Salado, Sabadell (ES)

Correspondence Address: KNOBBE MARTENS OLSON & BEAR LLP, 2040 MAIN STREET, FOURTEENTH FLOOR, IRVINE, CA, 92614, US

(21) Appl. No.: 12/376,283

(22) PCT Filed: Aug. 3, 2007
(86) PCT No.: PCT/EP2007/058047

§ 371 Date: Oct. 26, 2009

(30) Foreign Application Priority Data
Aug. 4, 2006 (EP) 06384013.6

(51) Int. Cl. A61K 31/5377; A61K 31/4545; A61K 31/454; C07D 413/08; C07D 401/08; A61P 25/00

(52) U.S. Cl. 514/235.2; 514/235.5; 514/235.8; 514/236.5; 514/316; 514/323; 514/326; 544/129; 544/139; 544/140; 544/144; 544/398; 546/191; 546/201; 546/210

(57) ABSTRACT

The present invention relates to substituted dimethylcyclobutyl compounds of general formula I, a process for their preparation, medicaments comprising said substituted dimethylcyclobutyl compounds as well as the use of said substituted dimethylcyclobutyl compounds for the preparation of medicaments, which are particularly suitable for the prophylaxis and/or treatment of disorders or diseases that are at least partially mediated via sigma receptors.

The present invention relates to substituted dimethylcyclobutyl compounds of general formula I,

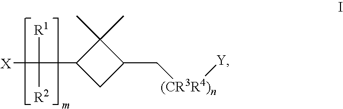

a process for their preparation, medicaments comprising said substituted dimethylcyclobutyl compounds as well as the use of said substituted dimethylcyclobutyl compounds for the preparation of medicaments, which are particularly suitable for the prophylaxis and/or treatment of disorders or diseases that are at least partially mediated via sigma receptors.

The search for new therapeutic agents has been greatly aided in recent years by better understanding of the structure of proteins and other biomolecules associated with target diseases. One important class of these proteins is the sigma receptor, a cell surface receptor of the central nervous system (CNS) which may be related to the dysphoric, hallucinogenic and cardiac stimulant effects of opioids.

From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease (Walker, J. M. et al, Pharmacological Reviews, 1990, 42, 355). It has been reported that the known sigma receptor ligand rimcazole clinically shows effects in the treatment of psychosis (Snyder, S. H., Largent, B. L. J. Neuropsychiatry 1989, 1, 7). The sigma binding sites have preferential affinity for the dextrorotatory isomers of certain opiate benzomorphans, such as (+)SKF 10047, (+)cyclazocine, and (+)pentazocine and also for some narcoleptics such as haloperidol.

The sigma receptor has at least two subtypes, which may be discriminated by stereoselective isomers of these pharmacoactive drugs. Possible sigma-site-mediated drug effects include modulation of glutamate receptor function, neurotransmitter response, neuroprotection, behaviour, and cognition (Quirion, R. et al. Trends Pharmacol. Sci., 1992, 13:85-86). Most studies have implied that sigma binding sites (receptors) are plasmalemmal elements of the signal transduction cascade. Drugs reported to be selective sigma ligands have been evaluated as antipsychotics (Hanner, M. et al. Proc. Natl. Acad. Sci., 1996, 93:8072-8077). The existence of sigma receptors in the CNS, immune and endocrine systems have suggested a likelihood that it may serve as link between the three systems.

Thus, there is a need to find compounds that have pharmacological activity towards the sigma receptor, being both effective and selective, and having good “drugability” properties, i.e. good pharmaceutical properties related to administration, distribution, metabolism and excretion.

Therefore an object of the present invention was to provide compounds that are particularly suitable as active ingredients in medicaments, especially in medicaments for the prophylaxis and/or treatment of disorders or diseases related to sigma receptors, preferably sigma-1 receptors, such as food intake related disorders or pain.

Surprisingly, it has been found that the substituted dimethylcyclobutyl compounds of general formula I given below show good to excellent affinity for sigma receptors, in particular they show good to excellent affinity to sigma-1 receptors. These compounds are therefore particularly suitable as pharmacologically active agents in a medicament for the prophylaxis and/or treatment of disorders or diseases related to sigma receptors, preferably related to sigma-1 receptors.

Thus, in one of its aspects the present invention relates to a dimethylcyclobutyl compound of general formula I,

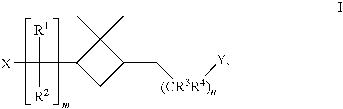

wherein

m is 1, 2, 3 or 4;

n is 0, 1, 2 or 3;

X represents a —OR⁵ moiety or a —NR⁶R⁷ moiety;

a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

Y represents a —OR⁸ moiety; a —NR⁸R¹⁰ moiety; a —C(═O)—OR¹¹ moiety;

a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

R¹ represents a hydrogen atom or a linear or branched, saturated or unsaturated, unsubstituted or at least mono-substituted aliphatic radical;

R² represents a hydrogen atom;

a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

R³ and R⁴, independently of one another, each represent a hydrogen atom or a linear or branched, saturated or unsaturated, unsubstituted or at least mono-substituted aliphatic radical;

R⁵ and R⁸, independently of one another, each represent a hydrogen atom or a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

or a —C(═O)—R¹² moiety;

R⁶, R⁷, R⁹ and R¹⁰, independently of one another, each represent a hydrogen atom or a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;

or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;

and

R¹¹ and R¹², independently of one another, represent a hydrogen atom or a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Preferred is a compound of general formula I, wherein

X and Y are different; wherein

if R⁶ represents hydrogen and m is 1,

R¹ represents a linear or branched, saturated or unsaturated, unsubstituted or at least mono-substituted aliphatic radical;

R² represents a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system; or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

wherein

R⁶, R⁷, R⁹ and R¹⁰, independently of one another, each represent a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;

or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;

or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;

R11 represents a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system; and

R¹² represents a hydrogen atom or a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system.

If any of the substituents in any of the above defined formulae represents or comprises a 3-, 4-, 5-, 6-, 7-, 8-, 9-, 10-, 11-, 12-, 13- or 14-membered heterocyclic ring, 3- to 9-membered (hetero)cycloaliphatic radical, C₃₋₉ cycloalkyl radical or C₄₋₉ cycloalkenyl radical, said heterocyclic ring, (hetero)cycloaliphatic radical, C₃₋₉ cycloalkyl radical or C₄₋₉ cycloalkenyl radical may—if not defined otherwise—be unsubstituted or substituted by one or more substituents, preferably unsubstituted or optionally substituted with 1, 2, 3, 4 or 5 substituent(s). Said substituent(s) may preferably be selected independently from the group consisting of oxo (═O), thioxo (═S), C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂—phenyl, —O—CFH₂, —O—CF₂H, —S—CFH₂, —S—CF₂H, —SO₃H, —NH—C(═O)—C₁₋₅-alkyl, —N(C₁₋₅-alkyl)—C(═O)—C₁₋₅-alkyl, —C(═O)—C₁₋₅-perfluoroalkyl, —S(═O)₂—NH₂, —S(═O)₂—NH—C₁₋₅-alkyl, —S(═O)₂—NH-phenyl, —NH—S(═O)₂—C₁₋₅-alkyl, cyclobutyl, cyclopropyl, cyclopentyl, cyclohexyl, pyrrolidinyl, piperidinyl, morpholinyl, phenyl, phenethyl, phenoxy and benzyl; whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, —S(═O)₂-phenyl, phenyl, phenethyl, phenoxy and benzyl; whereby in each occurrence said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

If any of the substituents in any of the above defined formulae represents or comprises a cycloaliphatic radical, C₃₋₉ cycloalkyl radical or C₄₋₉ cycloalkenyl radical which contains one or more, preferably 1, 2 or 3 heteroatom(s) as ring member(s), unless defined otherwise, each of these heteroatom(s) may preferably be selected independently from the group consisting of N, O and S.

If any of the substituents in any of the above defined formulae represents or comprises a heterocyclic ring which contains one or more, preferably 1, 2 or 3, additional heteroatom(s) as ring member(s), unless defined otherwise, each of these heteroatom(s) may preferably be selected independently from the group consisting of N, O and S.

Suitable saturated or unsaturated, optionally at least one heteroatom as ring member containing cycloaliphatic radicals, C₃₋₉ cycloalkyl radicals or C₄₋₉ cycloalkenyl radicals may preferably be selected from the group consisting of cyclopropyl, cyclobutyl, cyclopentyl, cyclohexyl, cycloheptyl, cyclooctyl, cyclononyl, cyclopentenyl, cyclohexenyl, cycloheptenyl, cyclooctenyl, pyrrolidinyl, piperidinyl, piperazinyl, homopiperazinyl, morpholinyl, aziridinyl, azetidinyl, imidazolidinyl, thiomorpholinyl, pyrazolidinyl, tetrahydrofuranyl, tetrahydrothiophenyl, azepanyl, diazepanyl, azocanyl, (2,5)-dihydrofuranyl, (2,5)-dihydrothiophenyl, (2,3)-dihydrofuranyl, (2,3)-dihydrofuranyl, (2,5)-dihydro-1H-pyrrolyl, (2,3)-dihydro-1H-pyrrolyl, tetrahydrothiopyranyl, tetrahydropyranyl, (3,4)-dihydro-2H-pyranyl, (3,4)-dihydro-2H-thiopyranyl, (1,2,3,6)-tetrahydropyridinyl, (1,2,3,4)-tetrahydropyridinyl, (1,2,5,6)-tetrahydropyridinyl, [1,3]-oxazinanyl, hexahydropyrimidinyl, (5,6)-dihydro-4H-pyrimidinyl, oxazolidinyl, (1,3)-dioxanyl, (1,4)-dioxanyl and (1,3)-dioxolanyl.

Suitable saturated or unsaturated, optionally at least one heteroatom as ring member containing cycloaliphatic radicals, C₃₋₉ cycloalkyl radicals or C₄₋₉ cycloalkenyl radicals which are condensed with an unsubstituted or at least mono-substituted mono- or bicyclic ring system may preferably be selected from the group consisting of indolinyl, isoindolinyl, decahydronaphthyl, (1,2,3,4)-tetrahydroquinolinyl, (1,2,3,4)-tetrahydroisoquinolinyl, (1,2,3,4)-tetrahydronaphthyl, octahydro-cyclopenta[c]pyrrolyl, (1,3,4,7,9a)-hexahydro-2H-quinolizinyl, (1,2,3,5,6,8a)-hexahydro-indolizinyl, decahydroquinolinyl, dodecahydrocarbazolyl, 9H-carbazolyl, decahydroisoquinolinyl, (6,7)-dihydro-4H-thieno[3,2-c]pyridinyl, (2,3)-dihydro-1H-benzo[de]isoquinolinyl, fluorenyl and (1,2,3,4)-tetrahydroquinoxazlinyl.

Suitable saturated, unsaturated or aromatic heterocyclic rings may preferably be selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, pyrrolyl, imidazolyl, pyrazolyl and triazolyl.

Suitable saturated, unsaturated or aromatic heterocyclic rings which are condensed with an unsubstituted or at least mono-substituted mono- or bicyclic ring system may preferably be selected from the group consisting of indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl and (4,5,6,7)-tetrahydro-1H-indolyl.

If any of the substituents in any of the above defined formulae represents an alkylene group, preferably an C₁₋₆alkylene group, an alkenylene group, preferably an C₂₋₆ alkenylene group or an alkinylene group, preferably an C₂₋₆ alkinylene group, which may be substituted, said alkylene group, C₂₋₆ alkylene group, alkenylene group, C₂₋₆ alkenylene group, alkinylene group or C₂₋₆ alkinylene group may be unsubstituted or substituted by one or more substituents, preferably unsubstituted or optionally substituted with 1, 2 or 3 substituent(s). Said substituent(s) may preferably be selected independently from the group consisting of —O—C₁₋₅-alkyl, —S—C₁₋₅-alkyl, —F, Cl, Br, I, —CN, —CF₃, —OCF₃, —SCF₃, —OH, —SH, —NH₂, —NH(C₁₋₅-alkyl) and —N(C₁₋₅-alkyl)₂, whereby in each occurrence C₁₋₅-alkyl may be linear or branched. An alkenylene group comprises at least one carbon-carbon double bond, an alkinylene group comprises at least one carbon-carbon triple bond.

Suitable alkylene groups include —(CH₂)—, —CH(CH₃)—, —CH(phenyl), —(CH₂)₂—, —(CH₂)₃—, —(CH₂)₄—, —(CH₂)₅ and —(CH₂)₆—, suitable alkenylene groups include —CH═CH—, —CH₂—CH═CH— and —CH═CH—CH₂— and suitable alkinylene groups include , —CH≡C—, —CH₂—C≡C— and —C≡C—CH₂—.

If any of the substituents in any of the above defined formulae represents or comprises an aryl radical, including a 6-membered aryl radical such as phenyl or a 10-membered aryl radical such as naphthyl or a 14-membered aryl radical such as anthracenyl, said aryl radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, —O—CFH₂, —O—CF₂H, —S—CFH₂, —S—CF₂H, —SO₃H, —NH—C(═O)—C₁₋₅-alkyl, —N(C₁₋₅)alkyl)—C(═O)—C₁₋₅-alkyl, —C(═O)—C₁₋₅-perfluoroalkyl, —S(═O)₂—NH₂, —S(═O)₂—NH—C₁₋₅-alkyl, —S(═O)₂—NH-phenyl, —NH—S(═O)₂—C₁₋₅-alkyl, cyclobutyl, cyclopropyl, cyclopentyl, cyclohexyl, pyrrolidinyl, piperidinyl, morpholinyl, phenyl, phenethyl, phenoxy and benzyl; whereby in each occurrence C₁₋₅-alkyl may be linear or branched and whereby said cyclic substituent(s) may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—C₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

Preferred aryl radicals, which may optionally be at least mono-substituted, are phenyl and naphthyl.

If any of the substituents in any of the above defined formulae represents or comprises a heteroaryl radical, including a monocyclic 5- or 6-membered heteroaryl radical or a bi- or tricyclic 8-, 9-, 10-, 11-, 12-, 13- or 14-membered heteroaryl radical, said heteroaryl radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, —O—CFH₂, —O—CF₂H, —S—CFH₂, —S—CF₂H, —SO₃H, —NH—C(═O)—C₁₋₅-alkyl, —N(C₁₋₅-alkyl)—C(═O)—C₁₋₅-alkyl, —C(═O)—C₁₋₅-perfluoroalkyl, —S(═O)₂—NH₂, —S(═O)₂—NH—C₁₋₅-alkyl, —S(═O)₂—NH-phenyl, —NH—S(═O)₂—C₁₋₅-alkyl, cyclobutyl, cyclopropyl, cyclopentyl, cyclohexyl, pyrrolidinyl, piperidinyl, morpholinyl, phenyl, phenethyl, phenoxy and benzyl; whereby in each occurrence C₁₋₅-alkyl may be linear or branched and whereby said cyclic substituent(s) may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

The heteroatom(s), which are present as ring member(s) in the heteroaryl radical, may, unless defined otherwise, independently be selected from the group consisting of nitrogen, oxygen and sulphur. Preferably the heteroaryl radical comprises 1, 2, 3 or 4 heteroatom(s).

Suitable bi- or tricyclic heteroaryl radicals, which may optionally be at least mono-substituted, may preferably be selected from the group consisting of indolyl, isoindolyl, quinolinyl, isoquinolinyl, benzo[b]furanyl, benzo[b]thiophenyl, benzo[2,1,3]thiadiazolyl, [1,2,3]-benzothiadiazolyl, [2,1,3]-benzoxadiazolyl, [1,2,3]-benzoxadiazolyl, benzoxazolyl, benzothiazolyl, benzisoxazolyl, benzisothiazolyl, imidazo[2,1-b]thiazolyl, 2H-chromenyl, indazolyl and quinazolinyl.

Suitable mono-, bi- or tricyclic heteroaryl radicals, which are condensed with an unsubstituted or at least mono-substituted saturated or unsaturated mono- or bicyclic ring system, may preferably be selected from the group consisting of (4,5,6,7)-tetrahydro-1H-indolyl, [1,3]-benzodioxolyl, [1,4]-benzodioxanyl, [1,2,3,4]-tetrahydronaphthyl, (2,3)-dihydro-1H-cyclopenta[b]indolyl, [1,2,3,4]-tetrahydroquinolinyl, [1,2,3,4]-tetrahydroisoquinolinyl, [1,2,3,4]-tetrahydroquinazolinyl and [3,4]-dihydro-2H-benzo[1,4]oxazinyl.

Suitable monocyclic heteroaryl radicals, which may optionally be at least mono-substituted, may preferably be selected from the group consisting of pyridinyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, pyrimidinyl, pyrazinyl and pyranyl.

A mono- or bicyclic ring system according to the present invention—if not defined otherwise—means a mono- or bicyclic hydrocarbon ring system that may be saturated, unsaturated or aromatic. Each of its different rings may show a different degree of saturation, i.e. it may be saturated, unsaturated or aromatic. Optionally each of the rings of the mono- or bicyclic ring system may contain one or more, preferably 1, 2 or 3, heteroatom(s) as ring member(s), which may be identical or different and which can preferably be selected from the group consisting of N, O and S. The rings of the mono- or bicyclic ring system are preferably 5-, 6- or 7-membered.

Preferably a mono-or bicyclic ring system according to the present invention is a phenyl or naphthyl ring system.

The term “condensed” according to the present invention means that a ring or ring system is attached to another ring or ring system, whereby the terms “annulated” or “annelated” are also used by those skilled in the art to designate this kind of attachment.

Such a mono- or bicyclic ring system may—if not defined otherwise—be unsubstituted or substituted by one or more substituents, preferably unsubstituted or substituted with 1, 2, 3, 4 or 5 substituent(s). Said substituents may preferably be selected independently from the group consisting of C₁₋₅-alkyl, —O—C₁₋₅-alkyl, —S—C₁₋₅-alkyl, —C(═O)—OH, oxo (═O), thioxo (═S), —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, —O—CFH₂, —O—CF₂H, —S—CFH₂, —S—CF₂H, —SO₃H, —NH—C(═O)—C₁₋₅-alkyl, —N(C₁₋₅-alkyl)—C(═O)—C₁₋₅-alkyl, —C(═O)—C₁₋₅-perfluoroalkyl, —S(═O)₂—NH₂, —S(═O)₂—NH—C₁₋₅-alkyl, —S(═O)₂—NH-phenyl, —NH—S(═O)₂—C₁₋₅-alkyl, cyclobutyl, cyclopropyl, cyclopentyl, cyclohexyl, pyrrolidinyl, piperidinyl, morpholinyl, phenyl, phenethyl, phenoxy and benzyl; whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, —S(═O)₂-phenyl, phenyl, phenethyl, phenoxy and benzyl; whereby in each occurrence said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

If any of the substituents in any of the above defined formulae represents a saturated or unsaturated aliphatic radical, i.e. an alkyl radical, preferably an C₁₋₁₀alkyl radical; an alkenyl radical, preferably an C₂₋₁₀ alkenyl radical or an alkinyl radical, preferably an C₂₋₁₀alkinyl radical; said aliphatic radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of —O—C₁₋₅-alkyl, —S—C₁₋₅-alkyl, F, Cl, Br, I, —CN, —CF₃, —OCF₃, —SCF₃, —OH, —SH, —NH₂, —NH(C₁₋₅-alkyl) and —N(C₁₋₅-alkyl)₂ , whereby in each occurrence C₁₋₅-alkyl may be linear or branched. More preferably said substituent(s) may preferably be selected independently from the group consisting of —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, F, Cl, Br, I, —CN, —CF₃, —OCF₃, —SCF₃, —OH, —SH, —NH₂, NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂.

An alkenyl radical comprises at least one carbon-carbon double bond, an alkinyl radical comprises at least one carbon-carbon triple bond.

Suitable alkyl radicals, which may be substituted by one or more substituents, may preferably be selected from the group consisting of methyl, ethyl, n-propyl, isopropyl, n-butyl, isobutyl, sec-butyl, tert-butyl, n-pentyl, neo-pentyl, n-hexyl, n-heptyl, n-octyl, n-nonyl and n-decyl.

Suitable alkenyl radicals, which may be substituted by one or more substituents, may preferably be selected from the group consisting of vinyl, 1-propenyl, 2-propenyl, 1-butenyl, 2-butenyl and 3-butenyl.

Suitable alkinyl radicals, which may be substituted by one or more substituents, may preferably be selected from the group consisting of ethinyl, 1-propinyl, 2-propinyl, 1-butinyl, 2-butinyl and 3-butinyl.

Preferred is a substituted dimethylcyclobutyl compound of general formula I, wherein the stereoisomers have the general formulae Ia or Ib or Ic or Id,

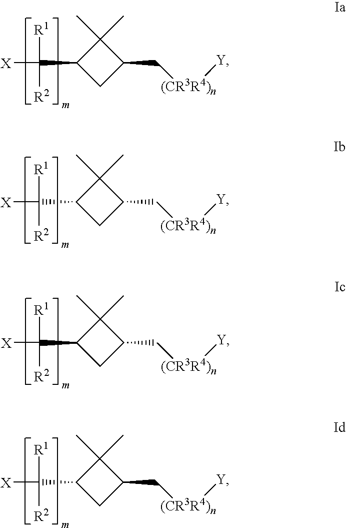

wherein

m, X, Y, R¹, R², R³, R⁴ and n have the above defined meanings.

Also preferred is a dimethylcyclobutyl compound of general formula I, wherein n is 0 or 1;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein m is 1;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

X represents a —OR⁵ moiety or a —NR⁶R⁷ moiety; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl, azepanyl (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—CH₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

Y represents a —OR⁸ moiety; a —NR⁹R¹⁰ moiety; a —C(═O)—OR¹¹ moiety; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃, or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R¹ represents a hydrogen atom or a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂CH₂—NH—C₂H₅;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R² represents a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅, a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—CH₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein R³ and R⁴ both represent a hydrogen atom;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R⁵ and R⁸, independently of one another, each represent a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of imidazolidinyl, aziridinyl, azetidinyl, pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl; which may be bonded via a —(CH₂)_{1, 2 or 3}-group and which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃ or a —C(═O)—R¹² moiety;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R⁶, R⁷, R⁹ and R¹⁰, independently of one another, each represent a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be bonded via a —(CH₂)_{1, 2 or 3}-group and which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, 13 C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R⁶ and R⁷ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl, phenoxy and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R⁹ and R¹⁹ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂—C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl, phenoxy and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Also preferred is a dimethylcyclobutyl compound of general formula I, wherein

R¹¹ and R¹², independently of one another, represent a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

and the other substituents have any of the above defined meanings, optionally in form of one of its stereoisomers, preferably enantiomers or diasteromers, a racemate or in form of a mixture of at least two of its stereoisomers, preferably enantiomers and/or diastereomers, in any mixing ratio, or a corresponding solvate thereof.

Particularly preferred is a dimethylcyclobutyl compound of general formula I, wherein

m is 1 or 2;

n is 0 or 1;

X represents a —OR⁵ moiety or a —NR⁶R⁷ moiety; or a (hetero)cycloaliphatic radical selected from the group consisting of pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH and —NH₂;

Y represents a —OR⁸ moiety; a —NR⁹R¹⁰ moiety; a —C(═O)—OR¹¹ moiety; or a (hetero)cycloaliphatic radical selected from the group consisting of pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—CH₂H₅, —C(═O)——CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH and —NH₂;

R¹ represents a hydrogen atom or a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH;

R² represents a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl and pyridinyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅ —S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H and —CFH₂;

R³ and R⁴ both represent a hydrogen atom;

R⁵ and R⁸, independently of one another, each represent a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH and —CH₂—SH; an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H, —CFH₂ and —S(═O)₂—CH₃ or a —C(═O)—R¹² moiety;

R⁶, R⁷, R⁹ and R¹⁰, independently of one another, each represent a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;

or R⁶ and R⁷ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃; phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

or R⁹ and R¹⁰ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

and R¹¹ and R¹², independently of one another, represent a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, and pyridinyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —S—CH₃, —S—C₂H₅, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

More particularly preferred is a dimethylcyclobutyl compound of general formula I, wherein

m is 1;

n is 0 or 1;

X represents a —OR⁵ moiety or a —NR⁶R⁷ moiety;

Y represents a —OR⁸ moiety; a —NR⁸R¹⁰ moiety or a —C(═O)—OR¹¹ moiety;

R¹ represents a hydrogen atom or a radical selected from the group consisting of methyl, ethyl, n-propyl, isopropyl, n-butyl, sec-butyl, isobutyl, tert-butyl and n-pentyl;

R² represents a hydrogen atom; a radical selected from the group consisting of methyl, ethyl, n-propyl, isopropyl, n-butyl, sec-butyl, isobutyl, tert-butyl and n-pentyl; or an aryl radical selected from the group consisting of phenyl and naphthyl, which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅—S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H and —CFH₂;

R³ and R⁴ both represent a hydrogen atom;

R⁵ and R⁸, independently of one another, each represent a hydrogen atom; a radical selected from the group consisting of methyl, ethyl, n-propyl, isopropyl, n-butyl, sec-butyl, isobutyl, tert-butyl and n-pentyl; an aryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅—F, Cl, Br, I, —CN, —CF₃, —OCF₃, —SCF₃, —OH, —SH, —NH₂, —NO₂, —CHO, —CF₂H, —CFH₂ and —S(═O)₂—CH₃ or a —C(═O)—R¹² moiety;

R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂and —S(═O)₂—CH₃;

or R⁶ and R⁷ together with the bridging nitrogen atom form a moiety selected from the group consisting of

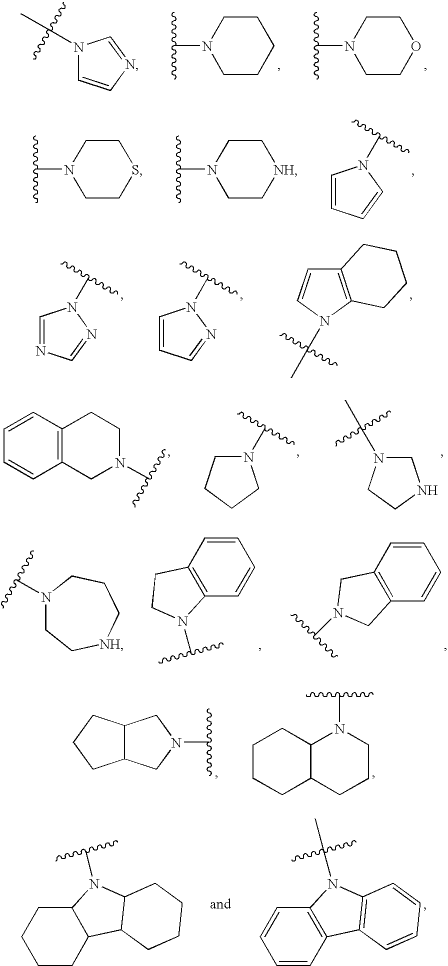

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

or R⁹ and R¹⁰ together with the bridging nitrogen atom form a moiety selected from the group consisting of

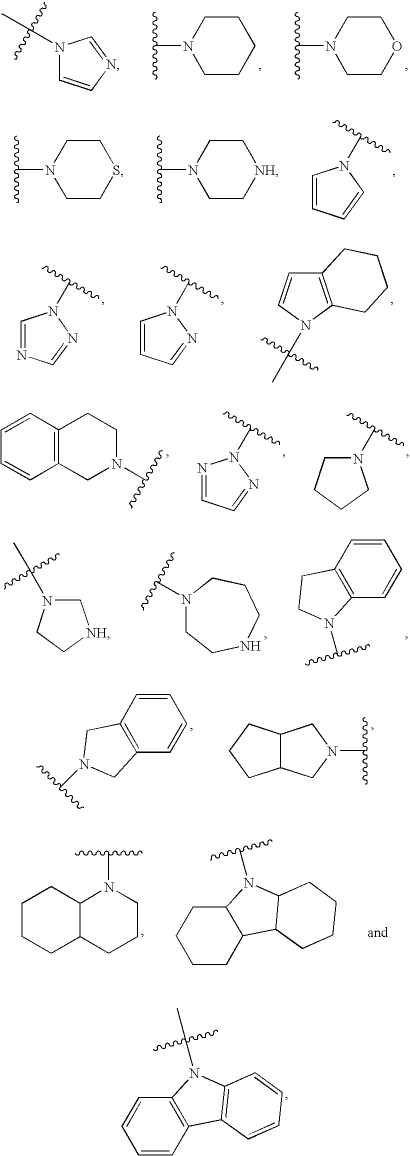

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

and R¹¹ and R¹², independently of one another, represent a hydrogen atom; or a radical selected from the group consisting of methyl, ethyl, n-propyl, isopropyl, n-butyl, sec-butyl, isobutyl, tert-butyl and n-pentyl;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Even more particularly preferred is a dimethylcyclobutyl compound of general formula I, wherein

m is 1;

n is 0 or 1;

X represents a —OR⁵ moiety or a —NR⁶R⁷ moiety;

Y represents a —OR⁸ moiety; a —NR⁹R¹⁰ moiety or a —C(═O)—OR¹¹ moiety;

R¹ represents a hydrogen atom or a radical selected from the group consisting of methyl, ethyl, n-propyl, isopropyl, n-butyl, sec-butyl, isobutyl, tert-butyl and n-pentyl;

R² represents a hydrogen atom; a radical selected from the group consisting of methyl, ethyl, n-propyl, isopropyl, n-butyl, sec-butyl, isobutyl, tert-butyl and n-pentyl; or a phenyl radical which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, F, Cl, Br, I, —CN, —CF₃, —OCF₃ and —SCF₃;

R³ and R⁴ both represent a hydrogen atom;

R⁵ and R⁸, independently of one another, each represent a hydrogen atom; a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅, F, Cl, Br, I, —CN, —CF₃ and —OCF₃or a —C(═O)—R¹² moiety;

R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, F, Cl, Br, I, —CN, —CF₃, —OCF₃ and —SCF₃;

or R⁶ and R⁷ together with the bridging nitrogen atom form a moiety selected from the group consisting of

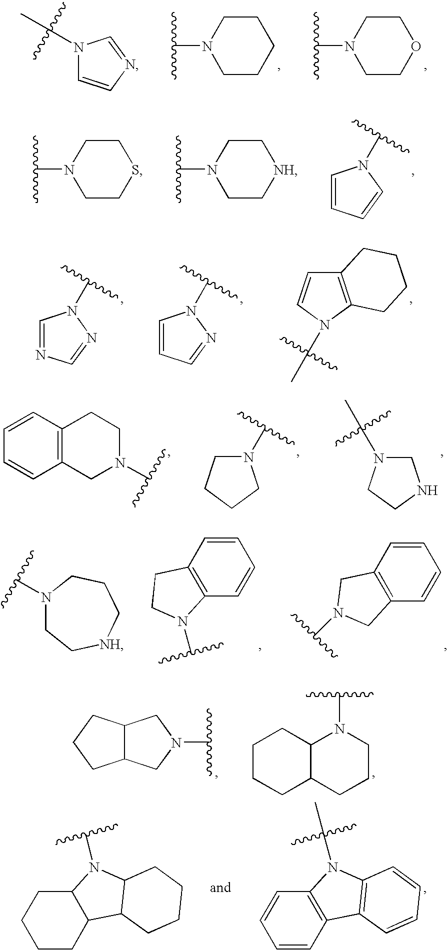

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

or R⁹ and R¹⁰ together with the bridging nitrogen atom form a moiety selected from the group consisting of

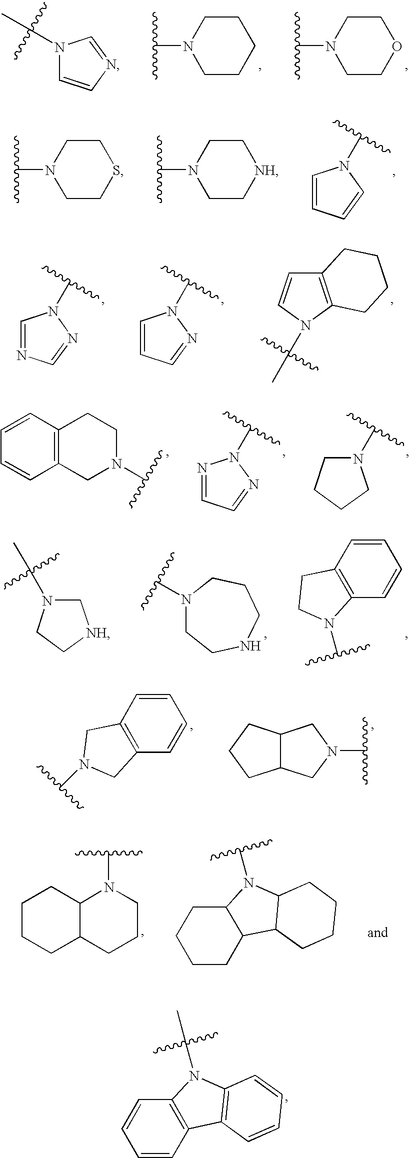

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

and R¹¹ and R¹², independently of one another, represent a radical selected from the group consisting of methyl, ethyl, n-propyl, isopropyl, n-butyl, sec-butyl, isobutyl, tert-butyl and n-pentyl;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Also even more particularly preferred is a dimethylcyclobutyl compound of general formula I, wherein

m is 1;

n is 0 or 1;

X represents a —OR⁵ moiety or a —NR⁶R⁷ moiety;

Y represents a —OR⁸ moiety; a —NR⁹R¹⁰ moiety or a —C(═O)—OR¹¹ moiety;

R¹ represents a hydrogen atom or a radical selected from the group consisting of methyl, ethyl, n-propyl, isopropyl, n-butyl, sec-butyl, isobutyl, tert-butyl and n-pentyl;

R² represents a hydrogen atom; a radical selected from the group consisting of methyl, ethyl, n-propyl, isopropyl, n-butyl, sec-butyl, isobutyl, tert-butyl and n-pentyl; or a phenyl radical which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, F, Cl, Br, I, —CN, —CF₃, —OCF₃ and —SCF₃;

R³ and R⁴ both represent a hydrogen atom;

R⁵ and R⁸, independently of one another, each represent a hydrogen atom; a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅, F, Cl, Br, I, —CN, —CF₃ and —OCF₃or a —C(═O)—R¹² moiety;

R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or a phenyl radical, which is bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, F, Cl, Br, I, —CN, —CF₃, —OCF₃ and —SCF₃;

or R⁶ and R⁷ together with the bridging nitrogen atom form a moiety selected from the group consisting of

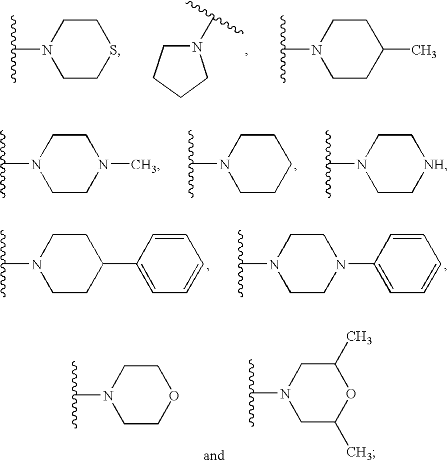

or R⁹ and R¹⁰ together with the bridging nitrogen atom form a moiety selected from the group consisting of

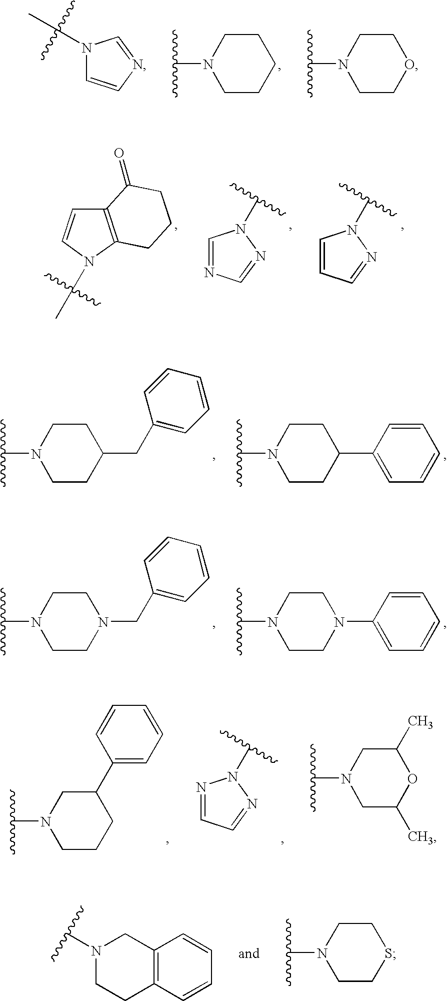

and R¹¹ and R¹², independently of one another, represent a radical selected from the group consisting of methyl, ethyl, n-propyl, isopropyl, n-butyl, sec-butyl, isobutyl, tert-butyl and n-pentyl;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Also preferred is a dimethylcyclobutyl compound of general formula Ie,

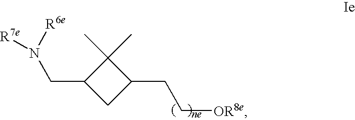

wherein

ne is 0 or 1;

R^{6e} and R^{7e}, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;

or R^{6e} and R^{7e} together with the bridging nitrogen atom form a moiety selected from the group consisting of

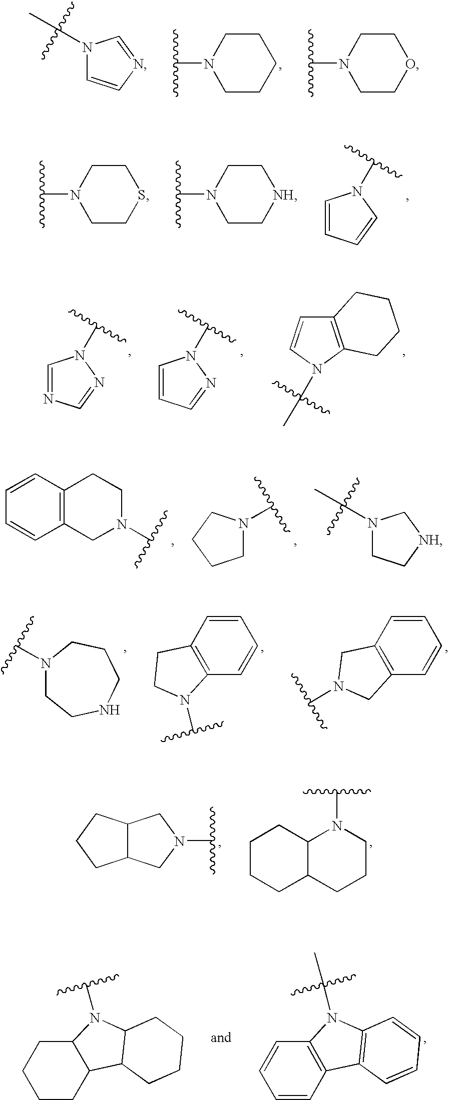

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

R^{8e} represents a hydrogen atom; a radical selected from the group consisting of methyl, ethyl, n-propyl, isopropyl, n-butyl, sec-butyl, isobutyl, tert-butyl and n-pentyl; an aryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅—F, Cl, Br, I, —CN, —CF₃, —OCF₃, —SCF₃, —OH, —SH, —NH₂, —NO₂, —CHO, —CF₂H, —CFH₂ and —S(═O)₂—CH₃ or a —C(═O)—R^{12e} moiety;

and R^{12e} represents a radical selected from the group consisting of methyl, ethyl, n-propyl, isopropyl, n-butyl, sec-butyl, isobutyl, tert-butyl and n-pentyl;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Preferred is a substituted dimethylcyclobutyl compound of general formula Ie, wherein the stereoisomers have the general formulae If or Ig or Ih or Ij,

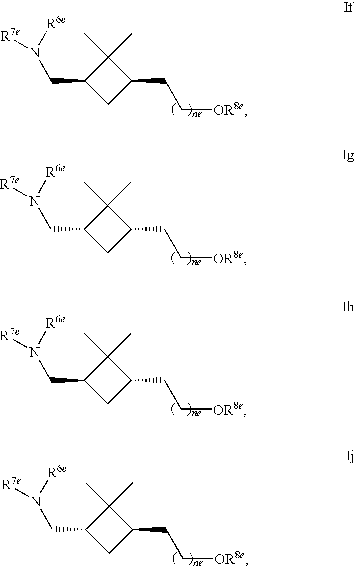

wherein ne, R^{6e}, R^{7e} and R^{8e} have the above defined meanings.

Also preferred is a dimethylcyclobutyl compound of general formula Ik,

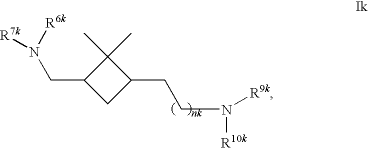

wherein

nk is 0 or 1;

R^{6k}, R^{7k}, R^{9k} and R^{10k}, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂and —S(═O)₂—CH₃;

or R^{6k} and R^{7k} together with the bridging nitrogen atom form a moiety selected from the group consisting of

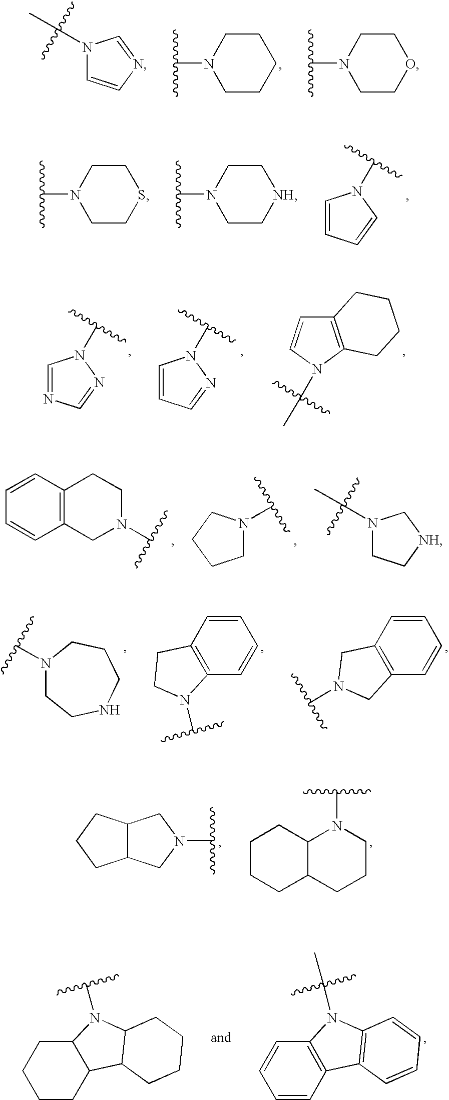

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, , phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

or R^{9k} and R^{10k} together with the bridging nitrogen atom form a moiety selected from the group consisting of

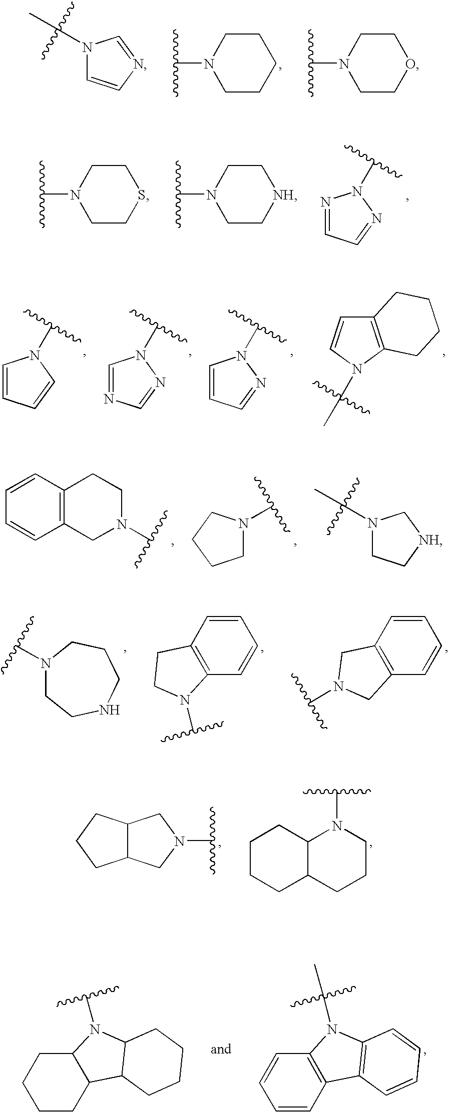

which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Preferred is a substituted dimethylcyclobutyl compound of general formula Ik, wherein the stereoisomers have the general formulae Im or In or Io or Ip,

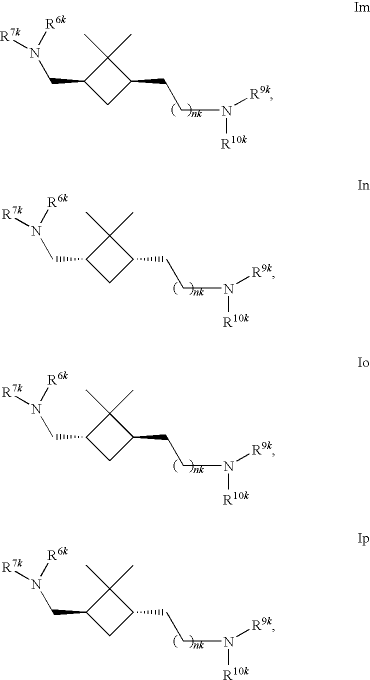

wherein nk, R^{6k}, R^{7k}, R^{9k} and R^{10k} have the above defined meanings.

Most particularly preferred is a dimethylcyclobutyl compound selected from the group consisting of

[1] 4-(((1S,3S)-3-(2-(benzyloxy)ethyl)-2,2-dimethylcyclobutyl)methyl)-morpholine

[2] 1-(((1S,3S)-3-(2-(benzyloxy)ethyl)-2,2-dimethylcyclobutyl)methyl)-piperidine

[3] 1-(((1S,3S)-3-(2-(benzyloxy)ethyl)-2,2-dimethylcyclobutyl)methyl)-4-phenylpiperidine

[4] 1-(((1S,3S)-3-(2-(benzyloxy)ethyl)-2,2-dimethylcyclobutyl)methyl)-4-phenylpiperazine

[5] 1-(((1S,3S)-3-(2-(benzyloxy)ethyl)-2,2-dimethylcyclobutyl)methyl)-4-methylpiperazine

[6] 4-(((1R,3S)-3-(benzyloxymethyl)-2,2-dimethylcyclobutyl)methyl)-morpholine

[7] 1-(((1R,3S)-3-(benzyloxymethyl)-2,2-dimethylcyclobutyl)methyl)-piperidine

[8] 1-(((1R,3S)-3-(benzyloxymethyl)-2,2-dimethylcyclobutyl)methyl)-4-phenylpiperidine

[9] 1-(((1R,3S)-3-(benzyloxymethyl)-2,2-dimethylcyclobutyl)methyl)-4-phenylpiperazine

[10] 1-(((1R,3S)-3-(benzyloxymethyl)-2,2-dimethylcyclobutyl)methyl)-4-methylpiperazine

[11] 1-((1R,3R)-2,2-dimethyl-3-(2-morpholinoethyl)cyclobutyl)ethanol

[12] 1-((1R,3R)-2,2-dimethyl-3-(2-(piperidin-1-yl)ethyl)cyclobutyl)ethanol

[13] 1-((1R,3R)-2,2-dimethyl-3-(2-morpholinoethyl)cyclobutyl)-1-phenylethanol

[14] 1-((1R,3R)-2,2-dimethyl-3-(2-(piperidin-1-yl)ethyl)cyclobutyl)-1-phenylethanol

[15] 4-(((1R,3R)-2,2-dimethyl-3-(2-phenoxyethyl)cyclobutyl)methyl)-morpholine

[16] 1-(((1R,3R)-2,2-dimethyl-3-(2-phenoxyethyl)cyclobutyl)methyl)piperidine

[17] 2-((1R,3R)-2,2-dimethyl-3-(piperidin-1-ylmethyl)cyclobutyl)ethanol

[18] 2-((1S,3S)-2,2-dimethyl-3-(piperidin-1-ylmethyl)cyclobutyl)ethanol

[19] 2-((1R,3R)-2,2-dimethyl-3-(morpholinomethyl)cyclobutyl)ethanol

[20] 2-((1S,3S)-2,2-dimethyl-3-(morpholinomethyl)cyclobutyl)ethanol

[21] 2-((1R,3R)-2,2-dimethyl-3-((4-methylpiperidin-1-yl)methyl)cyclobutyl)-ethanol

[22] 2-((1R,3R)-2,2-dimethyl-3-((4-phenylpiperidin-1-yl)methyl)cyclobutyl)-ethanol

[23] 2-((1R,3R)-3-(((2S,6R)-2,6-dimethylmorpholino)methyl)-2,2-dimethylcyclobutypethanol

[24] 2-((1R,3R)-3-(N-benzyl-N-methylamino)methyl)-2,2-dimethylcyclobutyl)-ethanol

[25] ((1R,3R)-3-(2-(4-benzylpiperidin-1-yl)ethyl)-2,2-dimethylcyclobutyl)-methanol

[26] methyl 2-((1R,3R)-2,2-dimethyl-3-(piperidin-1-ylmethyl)cyclobutyl)-acetate

[27] 1-(((1R,3R)-3-(2-(benzyloxy)ethyl)-2,2-dimethylcyclobutyl)methyl)-piperidine

[28] 1-(((1R,3R)-3-(2-(benzyloxy)ethyl)-2,2-dimethylcyclobutyl)methyl)-4-methylpiperidine

[29] 1-(((1R,3R)-3-(2-(benzyloxy)ethyl)-2,2-dimethylcyclobutyl)methyl)-4-phenylpiperidine

[30] 4-(((1R,3R)-3-(2-(benzyloxy)ethyl)-2,2-dimethylcyclobutyl)methyl)-thiomorpholine

[31] 4-(((1R,3R)-3-(2-(benzyloxy)ethyl)-2,2-dimethylcyclobutyl)methyl)-morpholine

[32] (2S,6R)-4-(((1R,3R)-3-(2-(benzyloxy)ethyl)-2,2-dimethylcyclobutyl)-methyl)-2,6-dimethylmorpholine

[33] 1-(((1R,3R)-3-(2-(benzyloxy)ethyl)-2,2-dimethylcyclobutyl)methyl)-pyrrolidine

[34] 1-(((1R,3R)-3-(2-(benzyloxy)ethyl)-2,2-dimethylcyclobutyl)methyl)-4-methylpiperazine

[35] 1-(((1R,3R)-3-(2-(benzyloxy)ethyl)-2,2-dimethylcyclobutyl)methyl)-4-methylpiperazine oxalate

[36] 1-(((1R,3R)-3-(2-(benzyloxy)ethyl)-2,2-dimethylcyclobutyl)methyl)-4-phenylpiperazine

[37] 1-(((1R,3R)-3-(2-(benzyloxy)ethyl)-2,2-dimethylcyclobutyl)methyl)-4-phenylpiperazine oxalate

[38] 1-(2-((1R,3R)-3-(benzyloxymethyl)-2,2-dimethylcyclobutyl)ethyl)-piperidine

[39] 4-(2-((1R,3R)-3-(benzyloxymethyl)-2,2-dimethylcyclobutyl)ethyl)-morpholine

[40] 4-(((1R,3R)-2,2-dimethyl-3-(2-(3-phenylpiperidin-1-yl)ethyl)cyclobutyl methyl)-morpholine

[41] 4-(((1R,3R)-2,2-dimethyl-3-(2-(4-phenylpiperidin-1-yl)ethyl)cyclobutyl methyl)morpholine

[42] 4-(((1R,3R)-3-(2-(4-benzylpiperidin-1-yl)ethyl)-2,2-dimethylcyclobutyl)-methyl)morpholine

[43] 4-(((1R,3R)-3-(2-(1H-imidazol-1-ypethyl)-2,2-dimethylcyclobutyl)-methyl)morpholine

[44] 4-(((1R,3R)-3-(2-(1H-pyrazol-1-ypethyl)-2,2-dimethylcyclobutyl)-methyl)morpholine

[45] 4-(((1R,3R)-3-(2-(1H-1,2,4-triazol-1-yl)ethyl)-2,2-dimethylcyclobutyly methyl)morpholine

[46] 1-(2-((1R,3R)-2,2-dimethyl-3-(morpholinomethyl)cyclobutyl)ethyl)-6,7-dihydro-1H-indo1-4(5H)-one

[47] 2-(2-((1R,3R)-2,2-dimethyl-3-(morpholinomethypcyclobutypethyl)-1,2,3,4-tetrahydroisoquinoline

[48] 1-(((1R,3R)-3-(2-(1H-imidazol-1-ypethyl)-2,2-dimethylcyclobutyl)-methyl)piperidine

[49] 1-(((1R,3R)-3-(2-(1H-pyrazol-1-yl)ethyl)-2,2-dimethylcyclobutyl)-methyl)piperidine

[50] 1-(((1R,3R)-3-(2-(1H-1,2,4-triazol-1-yl)ethyl)-2,2-dimethylcyclobutyl)-methyl)piperidine

[51] 1-(2-((1R,3R)-2,2-dimethyl-3-(piperidin-1-ylmethyl)cyclobutypethyl)-4-phenylpiperidine

[52] 4-benzyl-1-(2-((1R,3R)-2,2-dimethyl-3-(piperidin-1-ylmethyl)cyclobutyl)-ethyl)piperidine

[53] 1-(2-((1R,3R)-2,2-dimethyl-3-(piperidin-1-ylmethyl)cyclobutypethyl)-6,7-dihydro-1H-indol-4(5H)-one and

[54] 2-(2-((1R,3R)-2,2-dimethyl-3-(piperidin-1-ylmethyl)cyclobutypethyl)-1,2,3,4-tetrahydroisoquinoline;

[55] 2-((1R,3R)-2,2-Dimethyl-3-((piperidin-1-yl)methyl)cyclobutyl)ethyl pivalate

[56] 2-((1R,3R)-2,2-Dimethyl-3-((piperidin-1-yl)methyl)cyclobutyl)ethyl acetate

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

In still another aspect the present invention relates to a process for the preparation of a substituted dimethylcyclobutyl compound of general formula I, wherein at least one compound of general formula II,

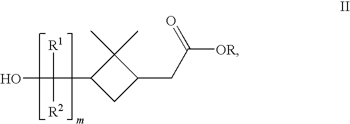

wherein m, R¹ and R² have the meaning given above and R represents a linear or branched C₁₋₅-alkyl radical, is reacted with methane sulfonylchloride, p-toluene sulfonylchloride, trifluormethane sulfonylchloride, thionyl chloride or tetrabromethane, preferably in a reaction medium selected from the group consisting of diethylether, tetrahydrofuran, tert-butylmethylether and dichloromethane or a mixture thereof, more preferably in dichloromethane, preferably in the presence of at least one base selected from the group consisting of pyridine, triethylamine, diisopropylamine, diisopropylethylamine, 4-methylmorpholine and morpholine, to yield at least one compound of general formula III,

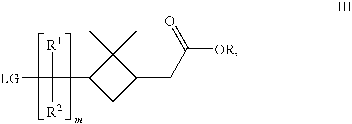

wherein m, R¹ and R² have the meaning given above, R represents a linear or branched C₁₋₅-alkyl radical and LG represents —O—S(═O)₂—CH₃, —O—S(═O)₂-p-toluyl, —O—S(═O)₂—CF₃, Cl or Br, which is optionally purified and/or isolated, and at least one compound of general formula III is reacted with at least one compound of general formula HNR⁶R⁷, wherein R⁶ and R⁷ have the meaning given above, preferably in a reaction medium selected from the group consisting of dimethylformamide, acetonitrile, dichloromethane and tetrahydrofuran or a mixture thereof, more preferably in tetrahydrofuran, preferably in the presence of at least one base selected from the group consisting of pyridine, triethylamine, diisopropylamine, diisopropylethylamine, 4-methylmorpholine and morpholine, to yield at least one compound of general formula IV,

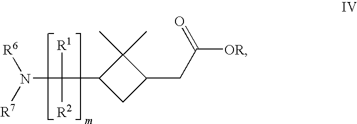

wherein m, R¹, R², R⁶ and R⁷ have the meaning given above and R represents a linear or branched C₁₋₅-alkyl radical, which is optionally purified and/or isolated,

and at least one compound of general formula IV is reacted with at least one reducing agent selected from the group consisting of lithium borohydride, sodium borohydride, lithium aluminium hydride and diborane, preferably with lithium borohydride, preferably in a reaction medium selected from the group consisting of methanol, ethanol, hexane and tetrahydrofuran or a mixture thereof, more preferably in methanol and/or tetrahydrofuran, to yield at least one compound of general formula V,

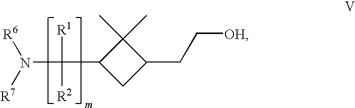

wherein m, R¹, R², R⁶ and R⁷ have the meaning given above, which is optionally purified and/or isolated,

and at least one compound of general formula V is reacted with methane sulfonylchloride, p-toluene sulfonylchloride, trifluormethane sulfonylchloride, thionyl chloride or tetrabromethane, preferably in a reaction medium preferably selected from the group consisting of tetrahydrofurane, diethylether, tert-butylmethylether and dichloromethane or a mixture thereof, more preferably in dichloromethane, preferably in the presence of at least one base selected from the group consisting of pyridine, triethylamine, diisopropylamine, diisopropylethylamine, 4-methylmorpholine and morpholine, to yield at least one compound of general formula VI,

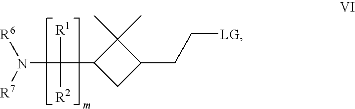

wherein m, R¹, R², R⁶ and R⁷ have the meaning given above and LG represents —O—S(═O)₂—CH₃, —O—S(═O)₂-p-toluyl, —O—S(═O)₂—CF₃, Cl or Br, which is optionally purified and/or isolated,

and at least one compound of general formula VI is reacted with at least one compound of general formula HNR⁹R¹⁰, wherein R⁹ and R¹⁰ have the meaning given above, preferably in a reaction medium selected from the group consisting of dimethylformamide, acetonitrile, dichloromethane and tetrahydrofuran or a mixture thereof, preferably in the presence of at least one base selected from the group consisting of pyridine, triethylamine, diisopropylamine, diisopropylethylamine, 4-methylmorpholine and morpholine, to yield at least one compound of general formula VII,

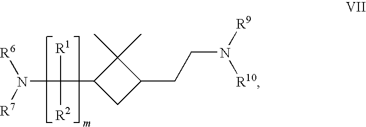

wherein m, R¹, R², R⁶, R⁷, R⁹ and R¹⁰ have the meaning given above, which is optionally purified and/or isolated,

or at least one compound of general formula VI is reacted with at least one compound of general formula M—OR⁸, wherein R⁸ has the meaning given above and M represents a monovalent kation selected from the group consisting of sodium, magnesium, potassium and lithium, preferably M represents a lithium kation, preferably in a reaction medium selected from the group consisting of dimethylformamide, dichloromethane and tetrahydrofuran or a mixture thereof, more preferably in tetrahydrofuran, preferably in the presence of at least one base selected from the group consisting of pyridine, triethylamine, diisopropylamine, diisopropylethylamine, 4-methylmorpholine and morpholine, to yield at least one compound of general formula VIII,

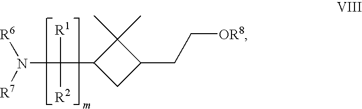

wherein m, R¹, R², R⁶, R⁷ and R⁸ have the meaning given above, which is optionally purified and/or isolated;

and optionally at least one compound of general formula VIII, wherein R⁸ represents hydrogen, is reacted with at least one compound of general formula LG—C(═O)—R¹², wherein R¹² has the above defined meaning and LG represents a leaving group, preferably a leaving group selected from the group consisting of chlorine and bromine, preferably in a reaction medium, preferably in the presence of at least one base selected from the group consisting of pyridine, triethylamine, diisopropylamine, diisopropylethylamine, 4-methylmorpholine and morpholine,

or with at least one compound of general formula HO—C(═O)—R¹², wherein R¹² has the above defined meaning, preferably in a reaction medium, preferably in the presence of at least one base selected from the group consisting of pyridine, triethylamine, diisopropylamine, diisopropylethylamine, 4-methylmorpholine and morpholine, preferably in the presence of at least one coupling agent,

to yield at least one compound of general formula VIIIa,

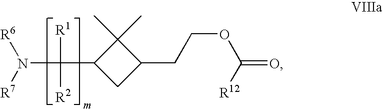

wherein m, R¹, R², R⁶, R⁷ and R¹² have the meaning given above, which is optionally purified and/or isolated.

In still another aspect the present invention relates to a process for the preparation of a substituted dimethylcyclobutyl compound of general formula I, wherein at least one compound of general formula II,

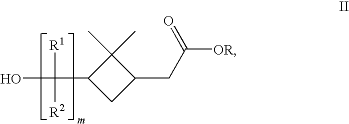

wherein m, R¹ and R² have the meaning given above and R represents a linear or branched C₁₋₅-alkyl radical, is reacted with at least one compound of general formula R⁵-LG, wherein R⁵ has the meaning given above and LG represents a leaving group, preferably LG represents a leaving group selected from the group consisting of chlorine, bromine, —O—S(═O)₂—CH₃, —O—S(═O)₂—CF₃ and —O—S(═O)₂-p-toluyl, more preferably LG represents bromine, preferably in a reaction medium selected from the group consisting of tetrahydrofuran, diethylether, tert-butylmethylether and dichloromethane or a mixture thereof, more preferably in dichloromethane, preferably in the presence of at least one base, more preferably in the presence of at least one base selected from the group consisting of sodium hydride, butyllithium, sodium ethoxide, potassium tert-butoxide and potassium hydride, to yield at least one compound of general formula IX,

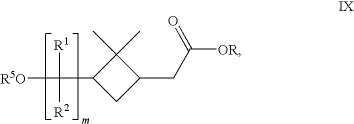

wherein m, R¹, R² and R⁵ have the meaning given above and R represents a linear or branched C₁₋₅-alkyl radical, which is optionally purified and/or isolated,

and at least one compound of general formula IX is reacted with at least one reducing agent selected from the group consisting of lithium borohydride, lithium aluminium hydride and diborane, sodium borohydride, preferably with lithium borohydride, preferably in a reaction medium selected from the group consisting of methanol, ethanol, hexane and tetrahydrofuran or a mixture thereof, more preferably in methanol and/or tetrahydrofuran, to yield at least one compound of general formula X,

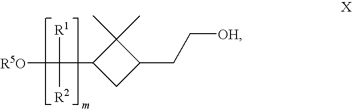

wherein m, R¹, R² and R⁵ have the meaning given above, which is optionally purified and/or isolated,

and optionally at least one compound of general formula X, is reacted with at least one compound of general formula LG—C(═O)—R¹², wherein R¹² has the meaning given above and LG represents a leaving group, preferably a leaving group selected from the group consisting of chlorine and bromine, preferably in a reaction medium, preferably in the presence of at least one base selected from the group consisting of pyridine, triethylamine, diisopropylamine, diisopropylethylamine, 4-methylmorpholine and morpholine,

or with at least one compound of general formula HO—C(═O)—R¹², wherein R¹² has the meaning given above, preferably in a reaction medium, preferably in the presence of at least one base, preferably in the presence of at least one coupling agent,

to yield at least one compound of general formula Xa,

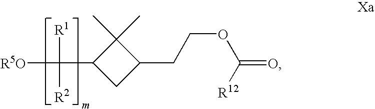

wherein m, R¹, R² and R⁵ have the meaning given above, which is optionally purified and/or isolated,

and at least one compound of general formula X is reacted with methane sulfonylchloride, p-toluene sulfonylchloride, trifluormethane sulfonylchloride, thionyl chloride or tetrabromethane, preferably in a reaction medium selected from the group consisting of diethylether, tetrahydrofuran, tert-butylmethylether and dichloromethane or a mixture thereof, more preferably in dichloromethane, preferably in the presence of at least one base selected from the group consisting of pyridine, triethylamine, diisopropylamine, diisopropylethylamine, 4-methylmorpholine and morpholine, to yield at least one compound of general formula XI,

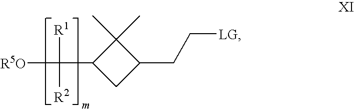

wherein m, R¹, R² and R⁵ have the meaning given above and LG represents —O—S(═O)₂—CH₃, —O—S(═O)₂-p-toluyl, —O—S(═O)₂—CF₃, Cl or Br, which is optionally purified and/or isolated,

and at least one compound of general formula XI is reacted with at least one compound of general formula HNR⁹R¹⁰, wherein R⁹ and R¹⁰ have the meaning given above, preferably in a reaction medium selected from the group consisting of dimethylformamide, acetonitrile, dichloromethane and tetrahydrofuran or a mixture thereof, preferably in the presence of at least one base selected from the group consisting of pyridine, triethylamine, diisopropylamine, diisopropylethylamine, 4-methylmorpholine and morpholine, to yield at least one compound of general formula XII,

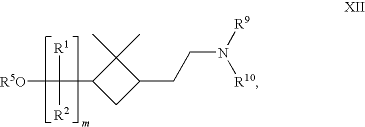

wherein m, R¹, R², R⁵, R⁹ and R¹⁰ have the meaning given above, which is optionally purified and/or isolated.

In still another aspect the present invention relates to a process for the preparation of a substituted dimethylcyclobutyl compound of general formula I, wherein at least one compound of general formula XIII,

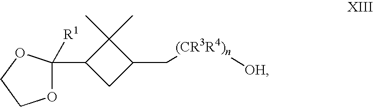

wherein R¹, R³ and R⁴ have the meaning given above, is reacted with methane sulfonylchloride, p-toluene sulfonylchloride, trifluormethane sulfonylchloride, thionyl chloride or tetrabromethane, preferably in a reaction medium selected from the group consisting of diethylether, tetrahydrofuran, tert-butylmethylether and dichloromethane or a mixture thereof, more preferably in dichloromethane, preferably in the presence of at least one base selected from the group consisting of pyridine, triethylamine, diisopropylamine, diisopropylethylamine, 4-methylmorpholine and morpholine,to yield at least one compound of general formula XIV,

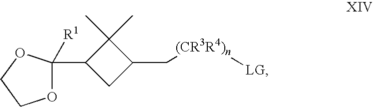

wherein R¹, R³ and R⁴ have have the meaning given above and LG represents —O—S(═O)₂—CH₃, —O—S(═O)₂-p-toluyl, —O—S(═O)₂—CF₃, Cl or Br, which is optionally purified and/or isolated,

and at least one compound of general formula XIV is reacted with at least one compound of general formula HNR⁹R¹⁰, wherein R⁹ and R¹⁰ have the meaning given above, preferably in a reaction medium selected from the group consisting of dimethylformamide, acetonitrile, dichloromethane and tetrahydrofuran or a mixture thereof, preferably in the presence of at least one base selected from the group consisting of pyridine, triethylamine, diisopropylamine, diisopropylethylamine, 4-methylmorpholine and morpholine, to yield at least one compound of general formula XV,

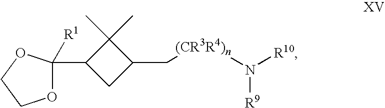

wherein R¹, R³, R⁴, R⁹ and R¹⁰ have the meaning given above, which is optionally purified and/or isolated,

and at least one compound of general formula XV is reacted with at least one reagent selected from the group consisting of hydrochloric acid, pyridinium p-toluenesulfonate, sulfonic acid and trifluoroacetic acid, preferably with pyridinium p-toluenesulfonate, preferably in a reaction medium selected from the group consisting of acetone and water or a mixture thereof, to yield at least one compound of general formula XVI,

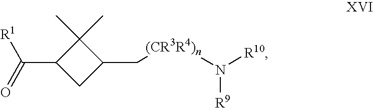

wherein R¹, R³, R⁴, R⁹ and R¹⁰ have the meaning given above, which is optionally purified and/or isolated,

and at least one compound of general formula XVI is reacted with at least one compound of general formula R²—Li, wherein R² has the meaning given above, or R²—Mg-Z, wherein R² has the meaning given above, and Z represents an anion selected from the group consisting of bromide and chloride, preferably in a reaction medium, selected from the group consisting of ether and tetrahydrofurane, to yield at least one compound of general formula XVII,

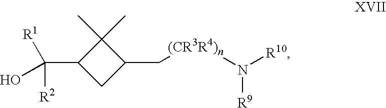

wherein R¹, R², R³, R⁴, R⁹ and R¹⁰ have the meaning given above, which is optionally purified and/or isolated,

and optionally at least one compound of general formula XVII is reacted with at least one compound of general formula R⁵-LG, wherein R⁵ has the meaning given above and LG represents a leaving group, preferably LG represents a leaving group selected from the group consisting of chlorine, bromine, —O—S(═O)₂—CH₃, —O—S(═O)₂—CF₃ and —O—S(═O)₂-p-toluyl, more preferably LG represents bromine, preferably in a reaction medium selected from the group consisting of tetrahydrofuran, diethylether, tert-butylmethylether and dichloromethane or a mixture thereof, more preferably in dichloromethane, preferably in the presence of at least one base, more preferably in the presence of at least one base selected from the group consisting of sodium hydride, butyllithium, sodium ethoxide, potassium tert-butoxide and potassium hydride, to yield at least one compound of general formula XVIII,

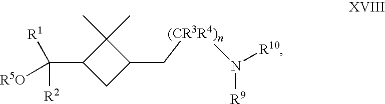

wherein R¹, R², R³, R⁴, R⁵, R⁹ and R¹⁰ have the meaning given above, which is optionally purified and/or isolated.

The compounds of general formulae I, Ia, Ib, Ic, Id, Ie, If, Ig, Ih, Ij, Ik, Im, In, Io and Ip given above may be purified and/or isolated according to methods well known to those skilled in the art. Preferably, the compounds of general formulae I, Ia, Ib, Ic, Id, Ie, If, Ig, Ih, Ij, Ik, Im, In, Io and Ip may be isolated by evaporating the reaction medium, addition of water and adjusting the pH value to obtain the compound in form of a solid that can be isolated by filtration, or by extraction with a solvent that is not miscible with water such as chloroform and purification by chromatography or recrystallisation from a suitable solvent.

The compounds of general formulae HNR⁶R⁷, HNR⁹R¹⁰, MOR⁸, R⁵-LG, R²—Li and R²—Mg-Z are commercially available or may also be prepared according to standard methods known in the prior art.

During some synthetic reactions described above or while preparing the compounds of general formulae I, Ia, Ib, Ic, Id, Ie, If, Ig, Ih, Ij, Ik, Im, In, Io and Ip the protection of sensitive or reactive groups may be necessary and/or desirable. This can be performed by using conventional protective groups like those described in Protective groups in Organic Chemistry, ed. J. F. W. McOmie, Plenum Press, 1973; T.W. Greene & P.G.M. Wuts and Protective Groups in Organic Chemistry, John Wiley & sons, 1993, 3^{rd} edition. The respective parts of the description is hereby incorporated by reference and forms part of the disclosure. The protective groups may be eliminated when convenient by means well-known to those skilled in the art.

If the substituted dimethylcyclobutyl compounds of general formulae I, Ia, Ib, Ic, Id, Ie, If, Ig, Ih, Ij, Ik, Im, In, Io and Ip are obtained in form of a mixture of stereoisomers, particularly enantiomers or diastereomers, said mixtures may be separated by standard procedures known to those skilled in the art, e.g. chromatographic methods or crystallization with chiral reagents.

The substituted dimethylcyclobutyl compounds of general formulae I, Ia, Ib, Ic, Id, Ie, If, Ig, Ih, Ij, Ik, Im, In, Io and Ip and in each case stereoisomers thereof may be obtained in form of a corresponding salt according to methods well known to those skilled in the art, e.g. by reacting said compound with at least one inorganic and/or organic acid, preferably in a suitable reaction medium. Suitable reaction media include, for example, any of the ones given above. Suitable inorganic acids include but are not limited to hydrochloric acid, hydrobromic acid, phosphoric acid, oxalic acid, sulfuric acid, nitric acid, suitable organic acids include but are not limited to citric acid, maleic acid, fumaric acid, tartaric acid, or derivatives thereof, p-toluenesulfonic acid, methanesulfonic acid or camphersulfonic acid.

The term “salt” is to be understood as meaning any form of the substituted dimethylcyclobutyl compounds in which they assume an ionic form or are charged and are coupled with a counter-ion (a cation or anion) or are in solution. By this are also to be understood complexes of the active compound with other molecules and ions, in particular complexes which are complexed via ionic interactions.

The term “physiologically acceptable salt” is understood in particular, in the context of this invention, as salt (as defined above) formed either with a physiologically tolerated acid, that is to say salts of the particular active compound with inorganic or organic acids which are physiologically tolerated—especially if used on humans and/or mammals - or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, hydrobromide, monohydrobromide, monohydrochloride or hydrochloride, methiodide, methanesulfonic acid, formic acid, acetic acid, oxalic acid, succinic acid, malic acid, tartaric acid, mandelic acid, fumaric acid, lactic acid, citric acid, glutamic acid, hippuric acid picric acid and/or aspartic acid. Examples of physiologically tolerated salts of particular bases are salts of alkali metals and alkaline earth metals and with NH₄.

Solvates, preferably hydrates, of the substituted dimethylcyclobutyl compounds of general formulae I, Ia, Ib, Ic, Id, Ie, If, Ig, Ih, Ij, Ik, Im, In, Io and Ip and in each case of corresponding stereoisomers may also be obtained by standard procedures known to those skilled in the art.

The term “solvate” according to this invention is to be understood as meaning any form of the substituted dimethylcyclobutyl compounds in which they have attached to it via non-covalent binding another molecule (most likely a polar solvent) especially including hydrates and alcoholates, e.g. ethanolate.

A further aspect of the present invention relates to a medicament comprising at least one substituted dimethylcyclobutyl compound of general formulae I, Ia, Ib, Ic, Id, Ie, If, Ig, Ih, Ij, Ik, Im, In, Io and Ip given above, optionally in form of one of its stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof, and optionally at least one physiologically acceptable auxiliary agent.

Said medicament is particularly suitable for sigma receptor regulation, preferably for sigma-1 receptor regulation, and can therefore be used for the prophylaxis and/or treatment of a disorder or a disease that is least partially mediated via sigma receptors, preferably sigma-1 receptors.

Preferably said medicament is suitable for the prophylaxis and/or treatment of a disorder or disease related to food intake, preferably for the regulation of appetite, for the maintenance, increase or reduction of body weight, for the prophylaxis and/or treatment of obesity, bulimia, anorexia, cachexia, type II diabetes (non insulin dependent diabetes mellitus), preferably type II diabetes that is caused by obesity; for the prophylaxis and/or treatment of diarrhoea; lipoprotein disorders, preferably selected from the group consisting of hypercholesterolemia (type II hyperlipoproteinemia); hypertriglyceridemia; hypoalphalipoproteinemia and high lipoprotein(a) levels; arthritis; hypertension; arrhythmia; ulcer; tardive dyskinesia; stress; cancer; stroke; ischemic stroke; migraine; epilepsy; anxiety; panic attacks; depression; cognitive disorders; cognitive dysfunction associated with psychiatric diseases; memory disorders; psychosis; schizophrenia; for the prophylaxis and/or treatment of drug addiction and/or withdrawal or for the prophylaxis and/or treatment of cocaine addiction and/or withdrawal.

Also preferably said medicament is suitable for the prophylaxis and/or treatment of pain, preferably neuropathic pain, allodynia, analgesia, causalgia, central pain, dysesthesia, hyperesthesia, hyperalgesia, hypoalgesia, hypoesthesia, or neuralgia, more preferably neuropathic pain, hyperalgesia or allodynia.

In another aspect the present invention relates to the use of at least one substituted dimethylcyclobutyl compound of general formulae I, Ia, Ib, Ic, Id, Ie, If, Ig, Ih, Ij, Ik, Im, In, Io and Ip given above, optionally in form of one of their stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof, for the preparation of a medicament suitable for sigma receptor regulation, preferably for sigma-1 receptor regulation and/or preferably for the prophylaxis and/or treatment of a disorder or a disease that is least partially mediated via sigma receptors, preferably sigma-1 receptors.

The use of at least one substituted dimethylcyclobutyl compound of general formulae I, Ia, Ib, Ic, Id, Ie, If, Ig, Ih, Ij, Ik, Im, In, Io and Ip given above, optionally in form of one of its stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof, for the manufacture of a medicament for the prophylaxis and/or treatment of a disorder or disease related to food intake, preferably for the regulation of appetite, for the maintenance, increase or reduction of body weight, for the prophylaxis and/or treatment of obesity, bulimia, anorexia, cachexia, type II diabetes (non insulin dependent diabetes mellitus), preferably type II diabetes that is caused by obesity; for the prophylaxis and/or treatment of diarrhoea; lipoprotein disorders, preferably selected from the group consisting of hypercholesterolemia (type II hyperlipoproteinemia); hypertriglyceridemia; hypoalphalipoproteinemia and high lipoprotein(a) levels; arthritis; hypertension; arrhythmia; ulcer; tardive dyskinesia; stress; cancer; stroke; ischemic stroke; migraine; epilepsy; anxiety; panic attacks; depression; cognitive disorders; cognitive dysfunction associated with psychiatric diseases; memory disorders; psychosis; schizophrenia; for the prophylaxis and/or treatment of drug addiction and/or withdrawal or for the prophylaxis and/or treatment of cocaine addiction and/or withdrawal is preferred.

The use of at least one substituted dimethylcyclobutyl compound of general formulae I, Ia, Ib, Ic, Id, Ie, If, Ig, Ih, Ij, Ik, Im, In, Io and Ip given above, optionally in form of one of its stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof, for the manufacture of a medicament for the prophylaxis and/or treatment of pain, preferably neuropathic pain, allodynia, analgesia, causalgia, central pain, dysesthesia, hyperesthesia, hyperalgesia, hypoalgesia, hypoesthesia, or neuralgia, more preferably neuropathic pain, hyperalgesia or allodynia, is preferred.

The use of at least one substituted dimethylcyclobutyl compound of general formulae I, Ia, Ib, Ic, Id, Ie, If, Ig, Ih, Ij, Ik, Im, In, Io and Ip given above, optionally in form of one of its stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof, as pharmacological tool or as anxiolytic or as immunosuppressant is preferred.

Any medicament according to the present invention may be in any form suitable for the application to humans and/or animals, preferably humans including infants, children and adults. The medicament can be produced by standard procedures known to those skilled in the art, e.g. from the table of contents of “Pharmaceutics: The Science of Dosage Forms”, Second Edition, Aulton, M. E. (ED. Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 y “The Theory and Practice of Industrial Pharmacy”, Lachman L., Lieberman H. And Kanig J. (Eds.), Lea & Febiger, Philadelphia (1986). The respective descriptions are hereby incorporated by reference and form part of the disclosure. The composition of the medicament may vary depending on the route of administration.

The medicament of the present invention may, for example, be administered parentally in combination with conventional injectable liquid carriers, such as water or suitable alcohols. Conventional pharmaceutical excipients for injection, such as stabilizing agents, solubilizing agents, and buffers, may be included in such injectable compositions. These medicaments may for example be injected intramuscularly, intraperitoneally, or intravenously.

Medicaments according to the present invention may also be formulated into orally administrable compositions containing one or more physiologically compatible carriers or excipients, in solid or liquid form. These compositions may contain conventional ingredients such as binding agents, fillers, lubricants, and acceptable wetting agents. The compositions may take any convenient form, such as tablets, pellets, granules, capsules, lozenges, aqueous or oily solutions, suspensions, emulsions, or dry powdered forms suitable for reconstitution with water or other suitable liquid medium before use, for immediate or retarded release. The multiparticulate forms, such as pellets or granules, may e.g. be filled into a capsule, compressed into tablets or suspended in a suitable liquid.

Suitable controlled release formulations, materials and methods for their preparation are known from the prior art, e.g. from the table of contents of “Modified-Release Drug Delivery Technology”, Rathbone, M. J. Hadgraft, J. and Roberts, M. S. (Eds.), Marcel Dekker, Inc., New York (2002); “Handbook of Pharmaceutical Controlled Release Technology”, Wise, D. L. (Ed.), Marcel Dekker, Inc. New York, (2000); “Controlled Drug Delivery”, Vol, I, Basic Concepts, Bruck, S. D. (Ed.), CRD Press Inc., Boca Raton (1983) y de Takada, K. and Yoshikawa, H., “Oral Drug Delivery”, Encyclopedia of Controlled Drug Delivery, Mathiowitz, E. (Ed.), John Wiley & Sons, Inc., New York (1999), Vol. 2, 728-742; Fix, J., “Oral drug delivery, small intestine and colon”, Encyclopedia of Controlled Drug Delivery, Mathiowitz, E. (Ed.), John Wiley & Sons, Inc., New York (1999), Vol. 2, 698-728. The respective descriptions are hereby incorporated by reference and form part of the disclosure.

Medicaments according to the present invention may also comprise an enteric coating, so that their dissolution is dependent on pH-value. Due to said coating the medicament can pass the stomach undissolved and the respective dimethylcyclobutyl compound is liberated in the intestinal tract. Preferably the enteric coating is soluble at a pH value of 5 to 7.5. Suitable materials and methods for the preparation are known from the prior art.

Typically, the medicaments according to the present invention may contain 1-60% by weight of one or more substituted dimethylcyclobutyl compounds as defined herein and 40-99% by weight of one or more auxiliary substances (additives).

The liquid oral forms for administration may also contain certain additives such as sweeteners, flavoring, preservatives, and emulsifying agents. Non-aqueous liquid compositions for oral administration may also be formulated, containing edible oils. Such liquid compositions may be conveniently encapsulated in e.g., gelatin capsules in a unit dosage amount.

The compositions of the present invention may also be administered topically or via a suppository.

The daily dosage for humans and animals may vary depending on factors that have their basis in the respective species or other factors, such as age, sex, weight or degree of illness and so forth. The daily dosage for humans may preferably be in the range from 1 to 2000 mg, preferably 1 to 1500 mg, more preferably 1 to 1000, even more preferably 1 to 500 mg, most preferably 1 to 100 mg of active substance to be administered during one or several intakes per day.

In the following methods for determining the pharmacological activity of the substituted dimethylcyclobutyl compounds are described.

Pharmacological Methods:

Some representative compounds of the invention were tested for their activity as sigma (sigma-1 and sigma-2) inhibitors. The following protocols were followed:

Sigma-1 Receptor Binding

Brain membrane preparation and binding assays for the sigma-1 receptor were performed as described (DeHaven-Hudkins et al., Characterization of the binding of [³H]-(+)-pentazocine to recognition sites in guinea pig brain, Eur. J. Pharmacol. 227, 371-378) with some modifications.

In brief, guinea pig brains were homogenized in 10 vols. (w/v) of Tris-HCl 50 mM, 0.32 M sucrose, pH 7.4, with a Kinematica Polytron PT 3000 at 15000 r.p.m. for 30 s. The homogenate was centrifuged at 1000 g for 10 min at 4° C. and the supernatants collected and centrifuged again at 48000 g for 15 min at 4° C. The pellet was resuspended in 10 volumes of Tris-HCl buffer (50 mM, pH 7.4), incubated at 37° C. for 30 min, and centrifuged at 48000 g for 20 min at 4° C. Following this, the pellet was resuspended in fresh Tris-HCl buffer (50 mM, pH 7.4) and stored on ice until use.

Each assay tube contained 10 μL of [³H]-(+)-pentazocine (final concentration of 0.5 nM), 900 μL of the tissue suspension to a final assay volume of 1 mL and a final tissue concentration of approximately 30 mg tissue net weight/mL. Non-specific binding was defined by addition of a final concentration of 1 M haloperidol. All tubes were incubated at 37° C. for 150 min before termination of the reaction by rapid filtration over Schleicher & Schuell GF 3362 glass fibre filters [previously soaked in a solution of 0.5% polyethylenimine for at least 1 h]. Filters were then washed with four times with 4 mL of cold Tris-HCl buffer (50 mM, pH 7.4). Following addition of scintillation cocktail, the samples were allowed to equilibrate overnight. The amount of bound radioactivity was determined by liquid scintillation spectrometry using a Wallac Winspectral 1414 liquid scintillation counter. Protein concentrations were determined by the method of Lowry et al. (Protein measurement with the Folin phenol reagent, J. Biol. Chem, 193, 265).

Sigma-2 Receptor Binding

Binding studies for sigma-2 receptor were performed as described (Radesca et al., Synthesis and Receptor Binding of Enantiomeric N—Substituted cis-N-[2-(3,4-Dichlorophenyl)ethyl]-2-(1-pyrrolidinypciclohexylamines as High-Affinity Receptor Ligands, J. Med. Chem. 34, 3065-3074) with some modifications. In brief, brains from sigma receptor type I (sigma-1) knockout mice (Langa, F., Codony X., Tovar V., Lavado A., Gimenez E., Cozar P., Cantero M., Dordal A., Hernandez E., Perez R., Monroy X., Zamanillo D., Guitart X., Montoliu L1., 2003, Generation and phenotypic analysis of sigma receptor type I (Sigmal) knockout mice, European Journal of Neuroscience, Vol. 18, 2188-2196) were homogenized in a volume of 10 mL/g tissue net weight of ice-cold 10 mM Tris-HCl, pH 7.4, containing 320 mM sucrose (Tris-sucrose buffer) with a Potter-Elvehjem homogenizer (10 strokes at 500 r.p.m.) The homogenates were then centrifuged at 1000 g for 10 min at 4° C., and the supernatants were saved. The pellets were resuspended by vortexing in 2 mL/g ice-cold Tris-sucrose buffer and centrifuged again at 1000 g for 10 min. The combined 1000 g supernatants were centrifuged at 31000 g for 15 min at 4° C. The pellets were resuspended by vortexing in 3 mL/g 10 mM Tris-HCl, pH 7.4, and the suspension was kept at 25° C. for 15 min. Following centrifugation at 31000 g for 15 min, the pellets were resuspended by gentle Potter Elvehjem homogenisation to a volume of 1.53 mL/g in 10 mM Tris-HCl pH 7.4.

The assay tubes contained 10 μL of [³H]-DTG (final concentration of 3 nM), 400 μL of the tissue suspension (5.3 mL/g in 50 mM Tris-HCl, pH 8.0) to a final assay volume of 0.5 mL. Non-specific binding was defined by addition of a final concentration of 1 M haloperidol. All tubes were incubated at 25° C. for 120 min before termination of the reaction by rapid filtration over Schleicher & Schuell GF 3362 glass fibre filters [previously soaked in a solution of 0.5% polyethylenimine for at least 1 h]. Filters were washed with three times with 5 mL volumes of cold Tris-HCl buffer (10 mM, pH 8.0). Following addition of scintillation cocktail samples were allowed to equilibrate overnight. The amount of bound radioactivity was determined by liquid scintillation spectrometry using a Wallac Winspectral 1414 liquid scintillation counter. Protein concentrations were determined by the method of Lowry et al. (Protein measurement with the Folin phenol reagent, J. Biol. Chem, 193, 265).

The present invention is illustrated below with the aid of examples. These illustrations are given solely by way of example and do not limit the general spirit of the present invention.

### EXAMPLES

A compound of general formula II, wherein both R¹ and R² represent hydrogen and R represents tert-butyl, can be prepared starting from (−)-α-pinene as depicted in scheme 1. below.

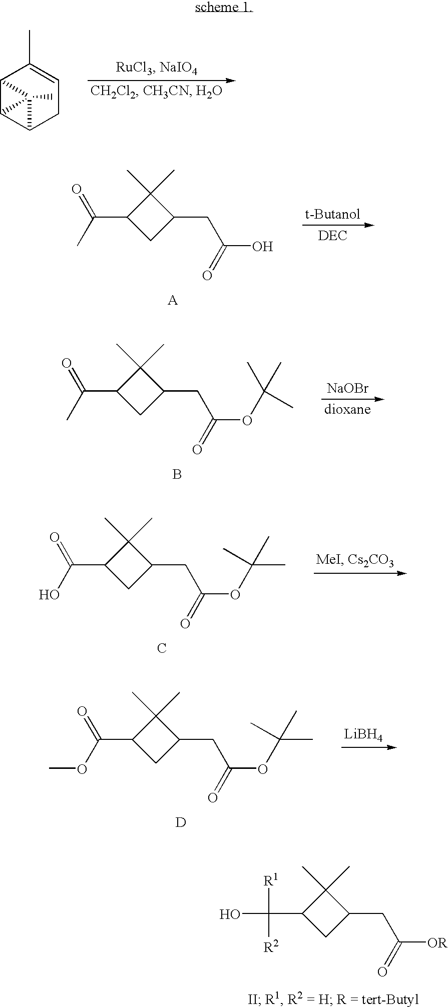

Exact reaction conditions for the each reaction step in scheme 1. are given in A. G. Moglioni et al. J. Org. Chem. 2000, 65, 3934-3940. The respective part of the literature is hereby incorporated by reference.

### Preparation of Compound C1

### (1R,3R)-3-((tert-Butoxycarbonyl)methyl)-2,2-dimethylcyclobutanecarboxylic acid

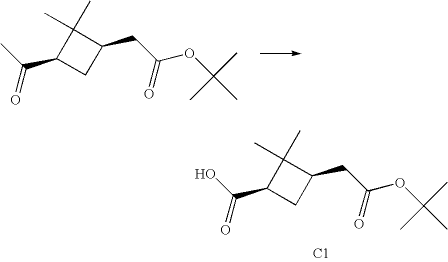

Aqueous NaOBr prepared from bromine (9.4 mL, 183.2 mmol), NaOH (28.4 g, 710.9 mmol) and water (237 mL) was added to a solution of tert-butyl 2-((1R,3R)-3-acetyl-2,2-dimethylcyclobutyl)acetate (5.5 g, 22.9 mmol) in 1,4-dioxane (138 mL) and water (38 mL). The reaction mixture was cooled to −5° C. and stirred at temperature between −5 and 0° C. for 5 h. The reaction mixture was washed with dichloromethane (4×200 mL). 40% aqueous NaHS₂O₃ was added to the basic aqueous layer at 0° C. until pH 5-6 was reached and the solution was acidified to pH 2 with conc. aqueous HCl. The acidic solution was extracted with dichloromethane (6×200 mL) and the organic extracts were dried over MgSO₄. The solvent was evaporated under reduced pressure to afford 4.9 g of (1R,3R)-3-((tert-butoxycarbonyl)methyl)-2,2-dimethylcyclobutanecarboxylic acid (90% yield), which was used in the next step without further purification.

¹H-NMR (250 MHz, CDCl₃)

1.0 (s, 3H), 1.3 (s, 3H), 1.5 (s, 9H), 2.0 (m, 1H), 2.1 (m, 1H), 2.3 (m, 2H), 2.5 (m, 1H), 2.8 (dd, J=10 Hz, J=10.5 Hz, 1H).

Compound C2 (1S,3S)-3-((tert-Butoxycarbonyl)methyl)-2,2-dimethylcyclobutanecarboxylic acid was prepared analogous to compound C1.

### Preparation of Compound D1

### Methyl (1R,3R)-3-((tert-butoxycarbonyl)methyl)-2,2-dimethylcyclobutane-carboxylate

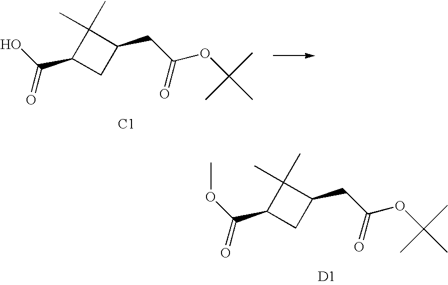

Cs₂CO₃ (7.9 g, 24.2 mmol) and Mel (1.5 mL, 24.2 mmol) were added to an ice-cooled solution of (1R,3R)-3-((tert-butoxycarbonyl)methyl)-2,2-dimethylcyclobutanecarboxylic acid (4.9 g, 20.2 mmol) in DMF (150 mL), and the reaction mixture was stirred at room temperature for 8 h. The reaction mixture was poured into EtOAc (200 mL) and the solution was washed with saturated aqueous NaHCO₃ (6×100 mL). The organic layer was dried over MgSO₄ and the solvents were removed under vacuo to afford 4.1 g of methyl (1R,3R)-3-((tert-butoxycarbonyl)methyl)-2,2-dimethylcyclobutanecarboxylate (80% yield), which was used in the next step without further purification.

¹H-NMR (250 MHz, CDCl₃)

0.9 (s, 3H), 1.2 (s, 3H), 1.5 (s, 9H), 2.0 (m, 1H), 2.1 (m, 1H), 2.3 (m, 2H), 2.4 (m, 1H), 2.8 (dd, J=10.5 Hz, J=11 Hz, 1H), 3.7 (s, 3H).

Compound D2 Methyl (1S,3S)-3-((tert-butoxycarbonyl)methyl)-2,2-dimethylcyclobutanecarboxylate was prepared analogous to compound D1.

### Preparation of Compounds of General Formula II

### tert-Butyl 2-((1R,3R)-3-(hydroxymethyl)-2,2-dimethylcyclobutyl)acetate

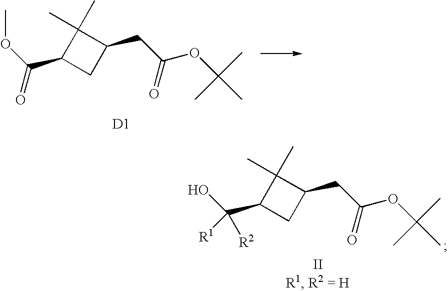

2 M LiBH₄ in THF (40 mL, 80.0 mmol) and dry MeOH (4 mL) was successively added to a solution of methyl (1R,3R)-3-((tert-butoxycarbonyl)methyl)-2,2-climethylcyclobutanecarboxylic acid (4.1 g, 16.0 mmol) in anhydrous THF under nitrogen atmosphere. The mixture was allowed to stand for 15 min then heated to reflux for 12 h. Excess hydride was destroyed with MeOH and water, and the solution was extracted with CH₂Cl₂ (4×150 mL). The organic extracts were dried over MgSO₄ and the solvents were evaporated at reduced pressure to afford tert-butyl 2-((1R,3R)-3-(hydroxymethyl)-2,2-dimethylcyclobutyl)acetate (2.9 g, 79% yield), which was used in the next step without further purification.

¹H-NMR (250 MHz, CDCl₃)

0.9 (s, 3H), 1.2 (s, 3H), 1.5 (s, 9H), 2.1 (m, 3H), 2.2 (m, 3H), 3.6 (m, 2H).

The compound tert-Butyl 2-((1S,3S)-3-(hydroxymethyl)-2,2-dimethylcyclobutyl)-acetate was prepared as described above.

### Preparation of Compounds of General Formula III

### ((1R,3R)-3-((tert-Butoxycarbonyl)methyl)-2,2-dimethylcyclobutyl)methyl methanesulfonate

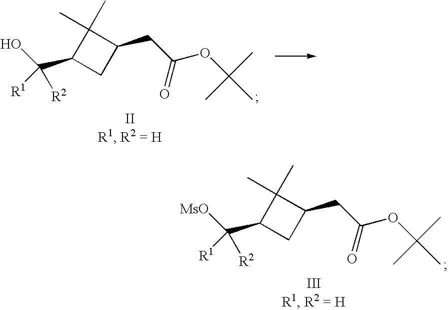

Triethylamine (1.5 mL, 10.4 mmol) and mesyl chloride (0.8 mL, 9.6 mmol) were added to an ice-cooled solution of tert-butyl 2-((1R,3R)-3-(hydroxymethyl)-2,2-dimethylcyclobutyl)acetate (1.7 g, 7.4 mmol) in anhydrous dichloromethane (70 mL) under nitrogen atmosphere. After stirring at 0° C. for 1 h, the reaction mixture was washed with saturated aqueous NaHCO₃ (4×50 mL) and dried over MgSO₄. The solvent was removed to afford ((1R,3R)-3-((tert-butoxycarbonyl)methyl)-2,2-dimethylcyclobutyl)methyl methanesulfonate (1.9 g, 83% yield) that was used in the next step without further purification.

¹H-NMR (250 MHz, CDCl₃)

1.0 (s, 3H), 1.1 (s, 3H), 1.5 (s, 9H), 2.2 (m, 6H), 3.0 (s, 3H), 4.2 (m, 2H).

The compounds ((1S,3S)-3-((tert-Butoxycarbonyl)methyl)-2,2-dimethylcyclobutypmethyl methanesulfonate and 2-((1R,3R)-2,2-Dimethyl-3-(2-methyl-1,3-dioxolan-2-yl)cyclobutypethyl methanesulfonate (compound of general formula (XIV) were prepared as described above.

### Preparation of Compounds of General Formula IV

### tert-Butyl 2-((1R,3R)- 2,2-dimethyl-3-(morpholinomethyl)cyclobutyl)acetate

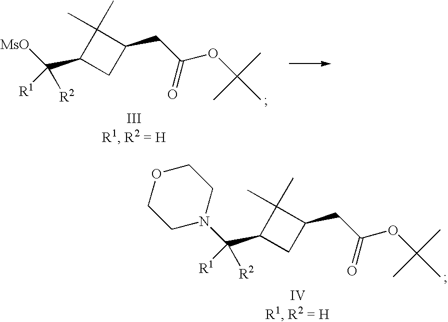

A mixture of ((1R,3R)- 3-((tentbutoxycarbonyl)methyl)-2,2-dimethylcyclobutyl)methyl methanesulfonate (1 g, 1.5 mmol) and morpholine (25 mL, 287 mmol) were heated at 80° C. for 7 days under nitrogen atmosphere. Then the mixture was cooled, EtOAc (25 mL) was added and the resultant solution was subsequently washed with saturated sodium NaHCO₃ and dried over MgSO₄. Solvent was removed to provide tert-butyl 2-((1R,3R)-2,2-dimethyl-3-(morpholinomethyl)cyclobutyl)acetate (920 mg, 95% yield).

¹H-NMR (250 MHz, CDCl₃)

0.9 (s, 3H), 1.1 (s, 3H), 1.5 (s, 9H), 2.2 (m, 5H), 2.4 (m, 7H), 3.7 (m, 4H).

The following compounds of general formula IV were prepared as described above:

tert-Butyl 2-((1S,3S)-2,2-dimethyl-3-(morpholinomethyl)cyclobutyl)acetate

tert-Butyl 2-((1R,3R)-2,2-Dimethyl-3-((piperidin-1-yl)methyl)cyclobutyl)acetate

¹H-NMR (250 MHz, CDCl₃)

0.9 (s, 3H), 1.1 (s, 3H), 1.4 (s, 9H), 1.6 (m, 6H), 2.1 (m, 8H), 2.3 (m, 4H).

tert-Butyl 2-((1S,3S)-2,2-Dimethyl-3-((piperidin-1-yl)methyl)cyclobutyl)acetate

### Preparation of Compounds of General Formula V

### 2-((1R,3R)-2,2-Dimethyl-3-(morpholinomethyl)cyclobutyl)ethanol

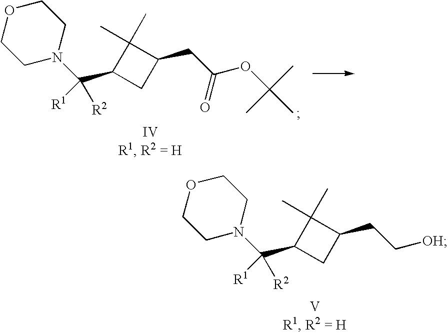

2 M LiBH₄ in THF (10 mL, 20.0 mmol) and dry methanol (1 mL) was successively added to a solution of tert-butyl 2-((1R,3R)-2,2-dimethyl-3-(morpholinomethyl)cyclobutyl)acetate (720 mg, 2.4 mmol) in anhydrous THF (20 mL) under nitrogen atmosphere. After 15 minutes standing, the mixture was heated to reflux for 24 h. Excess hydride was destroyed with methanol and the reaction mixture was poured into water. The resultant solution was extracted with CH₂Cl₂ (5×60 mL) and the organic extracts were dried over MgSO₄. Solvents were removed under reduced pressure to afford 700 g of a residue that was chromatographed on Baker® silica gel using 1% TEA-1:9 MeOH—CH₂Cl₂ as eluent to give pure 2-((1R,3R)-2,2-dimethyl-3-(morpholinomethyl)cyclobutyl)ethanol (410 mg, 75% yield).

¹H-NMR (250 MHz, CDCl₃)

0.9 (s, 3H), 1.1 (s, 3H), 1.6 (m, 3H), 1.9 (m, 1H), 2.2 (m, 3H), 2.4 (m, 5H), 3.6 (m, 2H), 3.7 (m, 4H).

¹³C-NMR (67.5 MHz, CDCl₃)

16.4 (1C), 30.1 (1C), 30.5 (1C), 33.4 (1C), 39.8 (1C), 39.9 (1C), 40.2 (1C), 53.9 (2C), 60.0 (1C), 61.6 (1C), 67.0 (2C).

MS (IFE-IT), m/z (%): 228.1 (M+1⁺,100)

The following products of general formulae and V and XVII were prepared as described above:

### 2-((1S,3S)-2,2-Dimethyl-3-(morpholinomethyl)cyclobutyl)ethanol

[a]=−23 (c=0.8, CH₂Cl₂)

### 2-((1R,3R)-2,2-Dimethyl-3-((piperidin-1-yl)methyl)cyclobutyl)ethanol

¹H-NMR (250 MHz, CDCl₃)

0.9 (s, 3H), 1.1 (s, 3H), 1.5 (m, 10H), 1.9 (m, 1H), 2.1 (m, 2H), 2.4 (m, 5H), 3.6 (m, 2H).

¹³C-NMR (67.5 MHz, CDCl₃)

16.4 (1C), 24.2 (1C), 25.8 (2C), 30.1 (1C), 31.3 (1C), 33.4 (1C), 40.0 (1C), 40.2 (1C), 40.4 (1C), 54.8 (2C), 60.2 (1C), 61.6 (1C).

MS (IFE-IT), m/z (%): 226.1 (M+1⁺,100)

[a]=+16 (c=0.3, CH₂Cl₂)

### 2-((1S,3S)-2,2-Dimethyl-3-((piperidin-1-yl)methyl)cyclobutyl)ethanol

[a]=−22 (c=1.2, CH₂Cl₂)

### 2-((1R,3R)-2,2-Dimethyl-3-((4-phenylpiperidin-1-yl)methyl)cyclobutyl)ethanol

¹H-NMR (250 MHz, CDCl₃)

1.0 (s, 3H), 1.1 (s, 3H), 1.6 (m, 3H), 1.8 (m, 5H), 2.1 (m, 7H), 2.5 (dd, J=12 Hz, 1H), 3.0 (m, 1H), 3.6 (m, 2H), 7.3 (m, 5H).

¹³C-NMR (67.5 MHz, CDCl₃)

16.4 (1C), 30.1 (1C), 31.2 (1C), 33.0 (1C), 33.2 (1C), 33.3 (1C), 39.9 (1C),

40.3 (1C), 40.7 (1C), 42.8 (1C), 54.3 (1C), 55.3 (1C), 60.0 (1C), 61.6 (1C), 126.2 (1C), 127.0 (2C), 128.3 (2C), 146.5 (1C).

MS (IFE-IT), m/z (%): 302.2 (M+1⁺,100)

[a]=+8 (c=1.3, CH₂Cl₂)

### 2-((1R,3R)-2,2-Dimethyl-3-(((2S,6R)-2,6-dimethylmorpholino)-methyl)cyclobutyl)ethanol

¹H-NMR (250 MHz, CDCl₃)

1.1 (s, 3H), 1.2 (s, 3H), 1.3 (d, J=4.5 Hz, 6H), 1.5 (m, 2H), 1.7 (m, 3H), 1.9 (m, 1H), 2.1 (m, 3H), 2.4 (dd, J=11.5 Hz, 1H), 2.7 (m, 2H), 3.6 (m, 2H), 3.7 (m, 2H).

MS (IFE-IT), m/z (%): 256.2 (M+1⁺,100)

### 2-((1R,3R)-2,2-Dimethyl-3-((4-methylpiperidin-1-yl)methyl)cyclobutyl)ethanol

¹H-NMR (250 MHz, CDCl₃)

0.9 (s, 3H), 0.9 (d, J=5 Hz, 3H), 1.1 (s, 3H), 1.5 (m, 2H), 1.7 (m, 4H), 1.9 (m, 4H), 2.1 (m, 4H), 2.5 (dd, J=15 Hz, J=12.5 Hz, 1H), 2.9 (m, 2H), 3.6 (m, 2H).

MS (IFE-IT), m/z (%): 240.1 (M+1⁺,100)

### 2-((1R,3R)-2,2-Dimethyl-3-(N-benzyl-N-methylamino)methyl)cyclobutyl)ethanol

¹H-NMR (250 MHz, CDCl₃)

0.9 (s, 3H), 1.1 (s, 3H), 1.6 (m, 4H), 1.9 (m, 1H), 2.1 (m, 2H), 2.2 (s, 3H), 2.5 (m, 1H), 3.4 (d, J=11.2 Hz, 1H), 3.5 (d, J=11.2 Hz, 1H), 3.6 (m, 2H), 7.3 (m, 5H).

MS (IFE-IT), m/z (%): 262.1 (M+1⁺,100)

### ((1R,3R)-3-(2-(4-Benzylpiperidin-1-yl)ethyl)-2,2-dimethylcyclobutyl)methanol

¹H-NMR (250 MHz, CDCl₃)

0.94 (s, 3H), 1.1 (s, 3H), 1.6 (m, 9H), 2.0 (m, 4H), 2.3 (m, 2H), 2.5 (d, J=6.7 Hz, 2H), 3.0 (m, 2H), 3.5 (m, 2H), 7.2 (m, 5H).

### Preparation of Compounds of General Formula VI

### 2-((1R,3R)-2,2-Dimethyl-3-(morpholinomethyl)cyclobutyl)ethyl methanesulfonate

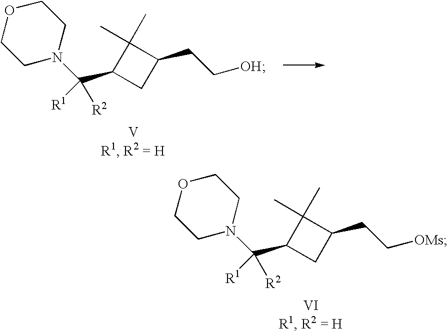

Triethylamine (1.2 mL, 8.7 mmol) and mesyl chloride 0.6 mL, 8.1 mmol) were added to an ice-cooled solution of alcohol 2-((1R,3R)-2,2-dimethyl-3-(morpholinomethyl)cyclobutyl)ethanol (1.4 g, 6.2 mmol) under nitrogen atmosphere. After 1 h stirring the reaction mixture was washed with saturated aqueous NaHCO₃ (4×50 mL), and the organic phase was dried over MgSO₄. The solvent was evaporated in vacuo to afford mesylate 2-((1R,3R)-2,2-Dimethyl-3-(morpholinomethyl)cyclobutyl)ethyl methanesulfonate (1.6 g, 85% yield) that was used in the next step without further purification.

¹H-NMR (250 MHz, CDCl₃)

1.0 (s, 3H), 1.1 (s, 3H), 1.6 (m, 2H), 1.9 (m, 2H), 2.2 (m, 3H), 2.4 (m, 5H), 3.0 (s, 3H), 3.7 (m, 4H), 4.2 (m, 2H).

The following product of general formula VI was prepared as described above:

### 2-((1R,3R)-2,2-Dimethyl-3-((piperidin-1-yl)methyl)cyclobutyl)ethyl methanesulfonate

¹H-NMR (250 MHz, CDCl₃)

0.9 (s, 3H), 1.1 (s, 3H), 1.4 (m, 2H), 1.6 (m, 6H), 1.8 (m, 2H), 2.1 (m, 3H), 2.4 (m, 5H), 3.0 (s, 3H), 4.2 (m, 2H).

### Preparation of Example Compound 46

### 1-(2-((1R,3R)-2,2-dimethyl-3-(morpholinomethyl)cyclobutyl)ethyl)-6,7-dihydro-1H-indol-4(5H)-one

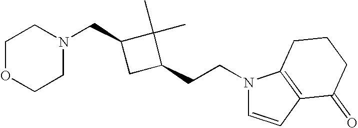

1.6 M BuLi in hexane (1.1 mL, 1.8 mmol) was added dropwise to an ice-cooled solution of 2-((1R,3R)-2,2-dimethyl-3-(morpholinomethyl)cyclobutyl)ethyl methanesulfonate (500 mg, 1.6 mmol) and 6,7-dihydro-1H-indol-4(5H)-one (245 mg, 1.8 mmol) in anhydrous DMF (45 mL) under nitrogen atmosphere. The light-protected mixture was heated to reflux for 9 days, then cooled to room temperature and diluted with EtOAc (60 mL). The resultant solution was washed with saturated aqueous NaHCO₃ and dried over MgSO₄. The solvents were removed at reduced pressure affording 750 mg of crude 1-(2-((1R,3R)-2,2-dimethyl-3-(morpholinomethyl)cyclobutypethyl)-6,7-dihydro-1H-indol-4(5H)-one, which was chromatographed on Baker® silica gel using 1% Triethylamine in 1:9 MeOH—CH₂Cl₂ as eluent to provide pure 1-(2-((1R,3R)-2,2-dimethyl-3-(morpholinomethyl)cyclobutypethyl)-6,7-dihydro-1H-indol-4(5H)-one (325 mg, 58% yield).

¹H-NMR (250 MHz, CDCl₃)

1.0 (s, 3H), 1.1 (s, 3H), 1.6 (m, 2H), 1.8 (m, 2H), 2.1 (m, 2H), 2.2 (m, 3H), 2.5 (m, 7H), 2.7 (m, 2H), 3.7 (m, 6H), 6.6 (s, 2H).

¹³C-NMR (67.5 MHz, CDCl₃)

16.2 (1C), 21.6 (1C), 23.5 (1C), 29.8 (1C), 30.0 (1C), 31.4 (1C), 37.5 (1C), 39.4 (1C), 40.0 (1C), 40.2 (1C), 45.1 (1C), 53.7 (2C), 59.6 (1C), 66.7 (2C), 105.3 (1C), 120.5 (1C), 121.9 (1C), 142.9 (1C), 194.2 (1C).

MS (IFE-IT), m/z (%): 345.2 (M+1⁺,100)

The following example compounds were prepared as described above:

### Example Compound 53

### 6,7-Dihydro-1-(2-((1R,3R)-2,2-dimethyl-3-((piperidin-1-yl)methyl)cyclobutyl)ethyl)-1H-indol-4(5H)-one

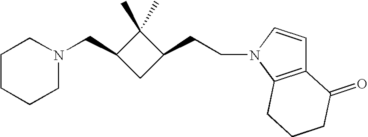

¹H-NMR (250 MHz, CDCl₃)

0.9 (s, 3H), 1.1 (s, 3H), 1.6 (m, 6H), 1.8 (m, 3H), 2.1 (m, 6H), 2.5 (m, 7H), 2.7 (m, 2H), 3.7 (m, 2H), 7.3 (s, 2H).

¹³C-NMR (67.5 MHz, CDCl₃)

16.5 (1C), 21.6 (1C), 22.7 (1C), 23.6 (2C), 23.7 (1C), 29.5 (1C), 30.9 (1C), 31.2 (1C), 37.6 (1C), 38.0 (1C), 40.6 (2C), 45.1 (1C), 53.8 (2C), 58.8 (1C), 105.4 (1C), 120.7 (1C), 121.9 (1C), 142.9 (1C), 194.0 (1C).

MS (IFE-IT), m/z (%): 343.2 (M+1⁺,100)

### Example Compound 42

### 4-(((1R,3R)-3-(2-(4-Benzylpiperidin-1-yl)ethyl)-2,2-dimethylcyclobutyl)methyl)morpholine

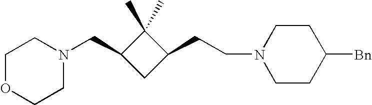

¹H-NMR (250 MHz, CDCl₃)

0.9 (s, 3H), 1.1 (s, 3H), 1.3 (m, 4H), 1.6 (m, 3H), 1.8 (m, 3H), 2.1 (m, 6H), 2.4 (m, 5H), 2.5 (d, J=7.8 Hz, 2H), 2.9 (m, 2H), 3.7 m, 4H), 7.2 (m, 3H), 7.3 (m, 2H).

MS (IFE-IT), m/z (%): 385.3 (M+1⁺,100)

### Example Compound 52

### 4-Benzyl-1-(2-((1R,3R)-2,2-dimethyl-3-((piperidin-1-yl)methyl)cyclobutyl)ethyl)piperidine

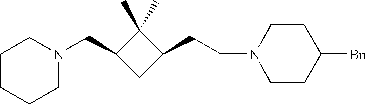

¹H-NMR (250 MHz, CDCl₃)

0.9 (s, 3H), 1.1 (s, 3H), 1.3 (m, 6H), 1.6 (m, 6H), 1.9 (m, 4H), 2.1 (m, 6H), 2.4 (m, 5H), 2.5 (d, J=8 Hz, 2H), 2.9 (m, 2H), 7.2 (m, 3H), 7.3 (m, 2H).

MS (IFE-IT), m/z (%): 383.3 (M+1⁺,100)

### Example Compound 41

### 4-(((1R,3R)-2,2-Dimethyl-3-(2-(4-phenylpiperidin-1-yl)ethyl)cyclobutyl)methyl)morpholine

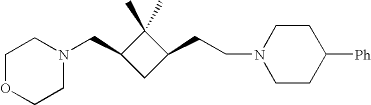

¹H-NMR (250 MHZ, CDCl₃)

1.0 (s, 3H), 1.1 (s, 3H), 1.5 (m, 1H), 1.6 (m, 1H), 1.8 (m, 6H), 2.1 (m, 7H), 2.4 (m, 7H), 3.1 (m, 1H), 3.7 (m, 4H), 7.3 (m, 5H).

MS (IFE-IT), m/z (%): 371.3 (M+1⁺,100)

### Example Compound 51

### 1-(2-((1R,3R)-2,2-Dimethyl-3-((piperidin-1-yl)methypcyclobutyl)ethyl)-4-phenylpiperidine

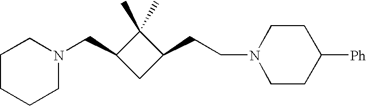

¹H-NMR (250 MHz, CDCl₃)

0.9 (s, 3H), 1.1 (s, 3H), 1.4 (m, 2H), 1.6 (m, 6H), 1.8 (m, 5H), 2.1 (m, 6H), 2.4 (m, 9H), 3.1 (m, 1H), 7.3 (m, 5H).

MS (IFE-IT), m/z (%): 369.3 (M+1⁺,100)

### Example Compound 40

### 4-(((1R,3R)-2,2-Dimethyl-3-(2-(3-phenylpiperidin-1-yl)ethyl)cyclobutyl)methyl)morpholine

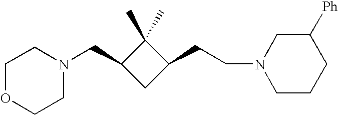

¹H-NMR (250 MHz, CDCl₃)

0.9 (s, 3H), 1.1 (s, 3H), 1.3 (m, 2H), 1.6 (m, 2H), 1.7 (m, 2H), 2.0 (m, 3H), 2.2 (m, 4H), 2.4 (m, 9H), 3.2 (m, 1H), 3.7 (m, 4H), 7.3 (m, 5H).

MS (IFE-IT), m/z (%): 371.2 (M+1⁺,100)

### Example Compound 43

### 4-(((1R,3R)-3-(2-(1H-Imidazol-1-yl)ethyl)-2,2-dimethylcyclobutyl)methyl)morpholine

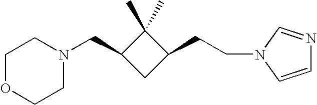

¹H-NMR (250 MHz, CDCl₃)

1.0 (s, 3H), 1.1 (s, 3H), 1.8 (m, 3H), 2.1 (m, 4H), 2.4 (m, 5H), 3.7 (m, 4H), 3.9 (m, 2H), 6.9 (s, 1H), 7.1 (s, 1H), 7.5 (s, 1H).

MS (IFE-IT), m/z (%): 278.1 (M+1⁺,100)

### Example Compound 48

### 1-(((1R,3R)-3-(2-(1H-Imidazol-1-yl)ethyl)-2,2-dimethylcyclobutyl)methyl)piperidine

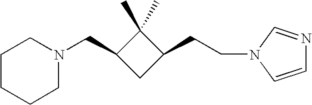

¹H-NMR (250 MHz, CDCl₃)

0.9 (s, 3H), 1.0 (s, 3H), 1.6 (m, 6H), 1.7 (m, 3H), 2.1 (m, 4H), 2.4 (m, 5H), 3.9 (m, 2H), 6.9 (s, 1H), 7.0 (s, 1H), 7.4 (s, 1H).

MS (IFE-IT), m/z (%): 276.2 (M+1⁺,100)

### Example Compound 44

### 4-(((1R,3R)-3-(2-(1H-Pyrazol-1-yl)ethyl)-2,2-dimethylcyclobutyl)methyl)morpholine

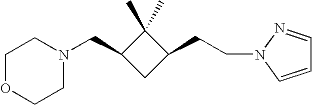

¹H-RMN (250 MHz, CDCl₃)

1.0 (s, 3H), 1.1 (s, 3H), 1.8 (m, 2H), 2.0 (m, 5H), 2.4 (m, 5H), 3.7 (m, 4H), 4.1 (m, 2H), 6.2 (t, J=2.5 Hz, 1H), 7.3 (d, J=2.5 Hz, 1H), 7.5 (d, J=2.5 Hz, 1H).

MS (IFE-IT), m/z (%): 278.1 (M+1⁺,100)

### Example Compound 49

### 1-(((1R,3R)-3-(2-(1H-Pyrazol-1-yl)ethyl)-2,2-dimethylcyclobutyl)methyl)piperidine

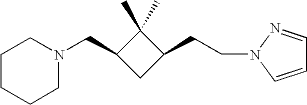

¹H-NMR (250 MHz, CDCl₃)

0.9 (s, 3H), 1.0 (s, 3H), 1.6 (m, 6H), 1.8 (m, 3H), 2.1 (m, 4H), 2.3 (m, 5H), 4.0 (m, 2H), 6.2 (t, J=2 Hz, 1H), 7.3 (d, J=2.8 Hz, 1H), 7.5 (d, J=1.6 Hz, 1H).

MS (IFE-IT), m/z (%): 276.2 (M+1⁺,100)

### Example Compound 45

### 4-(((1R,3R)-3-(2-(1H-1,2,4-Triazol-1-yl)ethyl)-2,2-dimethylcyclobutyl)methyl)morpholine

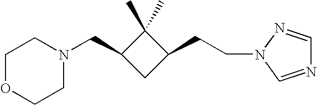

¹H-NMR (250 MHz, CDCl₃)

1.0 (s, 3H), 1.1 (s, 3H), 1.8 (m, 3H), 2.1 (m, 4H), 2.4 (m, 5H), 3.9 (m, 4H), 4.1 (m, 2H), 7.9 (s, 1H), 8.0 (s, 1H).

MS (IFE-IT), m/z (%): 279.1 (M+1⁺,100)

### Example Compound 50

### 1-(((1R,3R)-3-(2-(1H-1,2,4-Triazol-1-yl)ethyl)-2,2-dimethylcyclobutyl)methyl)piperidine

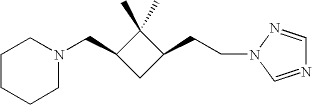

¹H-NMR (250 MHz, CDCl₃)

0.9 (s, 3H), 1.1 (s, 3H), 1.6 (m, 6H), 1.8 (m, 3H), 2.1 (m, 4H), 2.5 (m, 5H), 4.0 (m, 2H), 8.0 (s, 1H), 8.1 (s, 1H).

MS (IFE-IT), m/z (%): 277.2 (M+1⁺,100)

### Example Compound 47

### 1,2,3,4-Tetrahydro-2-(2-((1R,3R)-2,2-dimethyl-3-(morpholinomethyl)cyclobutyl)ethyl) isoquinoline

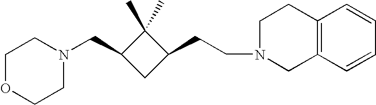

¹H-RMN (250 MHz, CDCl₃)

1.0 (s, 3H), 1.1 (s, 3H), 1.5 (m, 2H), 1.6 (m, 1H), 1.8 (m, 1H), 2.2 (m, 3H), 2.5 (m, 7H), 2.8 (m, 2H), 3.0 (m, 2H), 3.7 (s, 2H), 3.7 (m, 4H), 7.0 (m, 1H), 7.1 (m, 3H).

MS (IFE-1T), m/z (%): 343.3 (M+1⁺,100)

### Example Compound 54

### 1,2,3,4-Tetrahydro-2-(2-((1R,3R)-2,2-dimethyl-3-((piperidin-1-yl)methyl)cyclobutyl)ethylpsoquinoline

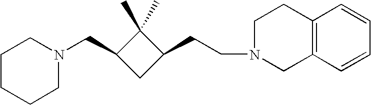

¹H-RMN (250 MHz, CDCl₃)

0.9 (s, 3H), 1.1 (s, 3H), 1.5 (m, 2H), 1.7 (m, 6H), 1.9 (m, 1H), 2.2 (m, 4H), 2.4 (m, 6H), 2.7 (m, 2H), 2.8 (s, 1H), 2.9 (m, 2H), 3.6 (m, 2H), 7.0 (m, 1H), 7.1 (m, 3H).

MS (IFE-IT), m/z (%): 341.3 (M+1⁺,100)

### Preparation of Example Compound 15

### 4-(((1R,3R)-2,2-Dimethyl-3-(2-phenoxyethyl)cyclobutyl)methyl)morpholine)

1.6 M n-BuLi in hexane (2.6 mL, 4.2 mmol) was added to an ice-cooled solution of phenol (350 mg, 3.7 mmol) in anhydrous THF (10 mL) under nitrogen atmosphere, and the mixture was stirred at 0° C. for 1 h. Then 2-((1R,3R)-2,2-dimethyl-3-(morpholinomethyl)cyclobutyl)ethyl methanesulfonate (500 mg, 1.6 mmol) in 20 mL THF was added and the mixture was subsequently heated to reflux for 6 days and evaporated to dryness. The residue was poured into EtOAc and the solution was washed with saturated aqueous NaHCO₃ and dried over MgSO₄. The solvent was removed under vacuo to give 879 mg of crude 4-(((1R,3R)-2,2-dimethyl-3-(2-phenoxyethyl)cyclobutyl)methyl)morpholine that was chromatographed twice on Baker® silica gel (1:40 MeOH—CH₂Cl₂ and CH₂Cl₂ as the respective eluents) giving 178 mg of 4-(((1R,3R)-2,2-dimethyl-3-(2-phenoxyethyl)cyclobutyl)methyl)morpholine (36% yield).

¹H-NMR (250 MHz, CDCl₃)

1.0 (s, 3H), 1.1 (s, 3H), 1.7 (m, 2H), 2.1 (m, 5H), 2.4 (m, 5H), 3.7 (m, 4H), 3.9 (m, 2H), 6.9 (m, 3H), 7.3 (m, 2H).

¹³C-NMR (67.5 MHz, CDCl₃)

16.6 (1C), 29.9 (1C), 30.1 (1C), 30.5 (1C), 39.8 (1C), 40.2 (1C), 40.3 (1C), 54.3 (2C), 60.0 (1C), 66.2 (1C), 66.7 (2C), 114.5 (2C), 120.5 (1C), 129.5 (2C), 159.2 (1C).

MS (IFE-IT), m/z (%): 304.2 (M+1⁺,100)

[a]=+13 (c=0.9, CH₂Cl₂)

### Example Compound 16

### 1-(((1R,3R)-2,2-Dimethyl-3-(2-phenoxyethyl)cyclobutyl)methyl)piperidine was prepared in a similar manner.

¹H-NMR (250 MHz, CDCl₃)

0.9 (s, 3H), 1.0 (s, 3H), 1.5 (m, 6H), 1.8 (m, 2H), 2.1 (m, 5H), 2.4 (m, 5H), 3.8 (m, 2H), 6.8 (m, 3H), 7.2 (m, 2H).

MS (IFE-IT), m/z (%): 302.2 (M+1⁺,100)

### Preparation of Example Compound 55

### 2-((1R,3R)-2,2-Dimethyl-3-((piperidin-1-yl)methyl)cyclobutyl)ethyl pivalate

Triethylamine (0.8 mL, 5.7 mmol) and pivaloyl chloride (0.6 mL, 5.2 mmol) were added to an ice-cooled solution of 2-((1R,3R)-2,2-dimethyl-3-(piperidin-1-ylmethyl)cyclobutyl)ethanol (0.9 g, 4.0 mmol) in anhydrous dichloromethane (50 mL) under nitrogen atmosphere. After stirring at 0° C. for 1 h, the reaction mixture was washed with saturated aqueous NaHCO₃ (4×25 mL) and dried over MgSO₄. The solvent was removed to afford 1.2 g of a residue that was cromatographed on Baker® silica gel using 1:9 MeOH—CH₂Cl₂ as eluent to give 2-((1R,3R)-2,2-dimethyl-3-(piperidin-1-ylmethyl)cyclobutyl)ethyl pivalate (300 mg, 24% yield). The compound was dissolved in hot dichloromethane and allowed to stand at room temperature for 96 h under pentane atmosphere. The produced precipitate was filtered and dried to afford 2-((1R,3R)-2,2-dimethyl-3-(piperidin-1-ylmethyl)cyclobutyl)ethyl pivalate (250 mg, 20% yield).

¹H-NMR (250 MHz, CDCl₃)

0.9 (s, 3H), 1.1 (s, 3H), 1.2 (s, 9H), 1.5 (m, 4H), 1.8 (m, 3H), 2.0 (m, 1H), 2.3 (m, 3H), 2.6 (m, 3H), 2.7 (m, 1H), 3.0 (m, 1H), 3.4 (m, 2H), 4.0 (m, 2H).

¹³C-NMR (67.5 MHz, CDCl₃)

16.5 (1C), 21.9 (2C), 22.5 (1C), 27.1 (3C), 28.8 (1C), 29.2 (1C), 31.5 (1C), 36.7 (1C), 38.6 (1C), 40.2 (1C), 40.9 (1C), 52.7 (1C), 53.6 (1C), 58.2 (1C), 62.5 (1C), 178.5 (1C).

MS (IFE-IT), m/z (%): 310.3 (M+1⁺,100)

[a]==16 (c=0.6, CH₂Cl₂)

mp=198° C. dec. (CH₂Cl₂/pentane)

The example compound 56 2-((1R,3R)-2,2-Dimethyl-3-((piperidin-1-yl)methypcyclobutypethyl acetate was prepared accordingly.

¹H-NMR (250 MHz, CDCl₃)

0.9 (s, 3H), 1.0 (s, 3H), 1.5 (m, 9H), 1.9 (m, 1H), 2.1 (s, 3H), 2.2 (m, 3H), 2.3 (m, 5H), 4.0 (m, 2H).

MS (IFE-IT), m/z (%): 268.2 (M+1⁺,100)

### Preparation of Compounds of General Formula IX

### tert-Butyl 2-((1R,3R)-3-((benzyloxy)methyl)-2,2-dimethylcyclobutypacetate

An ice-cooled mixture of tert-butyl 2-((1R,3R)-3-(hydroxymethyl)-2,2-dimethylcyclobutypacetate (2.5 g, 10.9 mmol) and 60% by weight NaH in mineral oil (3.2 g, 78.8 mmol) in DMF (100 mL) was stirred for 1.5 h under nitrogen atmosphere. Then benzyl bromide (7.3 mL, 61.4 mmol) was added dropwise keeping the temperature at 0 ° C. The light-protected mixture was allowed to warm and was stirred at room temperature for 72 h. The reaction mixture was diluted with wet EtOAc (100 mL) and water (40 mL) was carefully added. The resultant emulsion was washed with saturated aqueous NaHCO₃ (5×75 mL) and dried over MgSO₄. The solvents were removed under vacuo and the residue was lyophilized to afford tert-butyl 2-((1R,3R)-3-(benzyloxymethyl)-2,2-dimethylcyclobutyl)acetate (3.1 g, 85% yield) that was used in the next step without further purification.

¹H-NMR (250 MHz, CDCl₃)

0.9 (s, 3H), 1.1 (s, 3H), 1.5 (s, 9H), 2.2 (m, 6H), 3.5 (m, 2H), 4.5 (s, 2H), 7.4 (m, 5H).

### Preparation of Compounds of General Formula X

### 2-((1R,3R)-3-((Benzyloxy)methyl)-2,2-dimethylcyclobutyl)ethanol

2M LiBH₄ (19 mL, 20.0 mmol) and dry MeOH (1.9 mL) were successively added to a solution of tert-butyl 2-((1R,3R)-3-(benzyloxymethyl)-2,2-dimethylcyclobutyl)acetate (700 mg, 2.5 mmol) in anydrous THF under nitrogen atmosphere. The mixture was stirred at room temperature for 15 minutes then heated to reflux for 24 h. Excess hydride was destroyed with MeOH and the mixture was poured into water and extracted with CH₂Cl₂ (5×80 mL). The combined organic extracts were dried over MgSO4 and solvents were removed at reduced pressure to afford 2-((1R,3R)-3-(benzyloxymethyl)-2,2-dimethylcyclobutypethanol (500 mg, 79% yield) that was used in the next step without further purification.

¹H-NMR (250 MHz, CDCl₃)

1.0 (s, 3H), 1.1 (s, 3H), 1.6 (m, 2H), 2.0 (m, 4H), 3.4 (m, 2H), 3.6 (m, 2H), 4.5 (s, 2H), 7.4 (m, 5H).

### Preparation of Compounds of General Formula XI

### 2-((1R,3R)-3-((Benzyloxy)methyl)-2,2-dimethylcyclobutyl)ethyl methanesulfonate

An ice-cooled solution of alcohol 2-((1R,3R)-3-(benzyloxymethyl)-2,2-dimethylcyclobutyl)ethanol (500 mg, 2.0 mmol), mesyl chloride (0.2 mL, 2.6 mmol), triethylamine ((0.4 mL, 2.8 mmol) in anhydrous CH₂Cl₂ (10 mL) was stirred for 1 h under nitrogen atmosphere. The reaction mixture was washed with saturated aqueous NaHCO₃ (4×10 mL) and the organic phase was dried over MgSO₄. The solvent was removed at reduced pressure to afford crude mesylate 2-((1R,3R)-3-((Benzyloxy)methyl)-2,2-dimethylcyclobutyl)ethyl methanesulfonate (510 mg, 78% yield) that was used in the next step without further purification.

¹H-NMR (250 MHz, CDCl₃)

1.0 (s, 3H), 1.1 (s, 3H), 1.6 (m, 2H), 2.0 (m, 4H), 3.0 (s, 3H), 3.4 (m, 2H), 3.6 (m, 2H), 4.5 (s, 2H), 7.4 (m, 5H).

The compound 2-((1R,3R)-3-(1-hydroxyethyl)-2,2-dimethylcyclobutypethyl methanesulfonate was prepared accordingly.

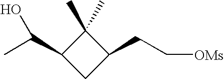

¹H-NMR (250 MHz, CDCl₃)

1.0 (s, 3H), 1.1 (d, J=5.5 Hz, 3H), 1.2 (s, 3H), 1.8 (m, 6H), 3.0 (s, 3H), 3.8 (m, 1H), 4.1 (m, 2H).

### Preparation of Example Compound 32

### (2S,6R)-4-(((1R,3R)-3-(2-(Benzyloxy)ethyl)-2,2-dimethylcyclobutyl)methyl)-2,6-dimethylmorpholine

A mixture of ((1R,3R)-3-(2-(benzyloxy)ethyl)-2,2-dimethylcyclobutyl)methyl methanesulfonate (500 mg, 1.5 mmol) and cis-(2R,6R)-dimethylmorpholine was heated to reflux for 24 h under nitrogen atmosphere. The reaction mixture was diluted with EtOAc (25 mL) and washed with saturated aqueous NaHCO₃. The organic layer was dried over MgSO₄ and the solvent was evaporated. The residue was chromatographed on Baker® silica gel (1:9 MeOH—CH₂Cl₂) to provide (2S,6R)-4-(((1R,3R)-3-(2-(benzyloxy)ethyl)-2,2-dimethylcyclobutyl)methyl)-2,6-dimethylmorpholine (429 mg, 81% yield).

¹H-NMR (250 MHz, CDCl₃)

0.9 (s, 3H), 1.1 (s, 3H), 1.2 (d, J=6 Hz, 6H), 1.6 (m, 4H), 2.0 (m, 5H), 2.4 (dd, J=10.5 Hz, 1H), 2.7 (m, 2H), 3.4 (m, 2H), 3.7 (m, 2H), 4.5 (s, 2H), 7.4 (m, 5H).

¹³C-NMR (67.5 MHz, CDCl₃)

16.4 (10), 19.1 (1C), 19.2 (1C), 30.1 (1C), 30.3 (10), 30.6 (1C), 39.9 (1C), 40.0 (1C), 40.1 (10), 59.5 (2C), 60.2 (1C), 69.0 (2C), 71.6 (1C), 72.9 (1C), 127.4 (10), 127.6 (2C), 128.3 (2C), 138.6 (1C).

MS (IFE-IT), m/z (%): 346.2 (M+1⁺,100)

[a]=+8 (c=2.3, CH₂Cl₂)

### Preparation of Example Compound 29

### 1-(((1R,3R)- 3-(2-(Benzyloxy)ethyl)-2,2-dimethylcyclobutyl)methyl)-4-phenylpiperidine

4-Phenylpiperidine (2.5 g, 15.5 mmol) was added to a solution of ((1R,3R)-3-(2-(benzyloxy)ethyl)-2,2-dimethylcyclobutyl)methyl methanesulfonate (250 mg, 0.8 mmol) in dry THF (15 mL) and the mixture was heated to reflux for 48 h under nitrogen atmosphere. The solvent was evaporated under vacuo and the residue was poured into EtOAc (25 mL) and successively washed with saturated aqueous NaHCO₃ and brine. The organic phase was dried over MgSO₄ and the solvent was removed. The residue (320 mg) was chromatographed on Baker® silica gel (1:9 MeOH—CH₂Cl₂) to afford pure 1-(((1R,3R)-3-(2-(benzyloxy)ethyl)-2,2-dimethylcyclobutypmethyl)-4-phenylpiperidine (144 mg, 50% yield).

¹H-NMR (250 MHz, CDCl₃)

0.9 (s, 3H), 1.1 (s, 3H), 1.7 (m, 9H), 2.1 (m, 6H), 2.5 (m, 1H), 3.0 (m, 1H), 3.4 (m, 2H), 4.5 (s, 2H), 7.4 (m, 10H).

¹³C-NMR (67.5 MHz, CDCl₃)

16.4 (1C), 30.1 (1C), 30.4 (1C), 31.2 (1C), 33.4 (1C), 33.5 (1C), 40.1 (1C), 40.2 (1C), 40.4 (1C), 42.6 (1C), 54.1 (1C), 55.8 (1C), 60.0 (1C), 69.1 (1C), 73.0 (1C), 126.0 (1C), 126.8 (2C), 127.5 (1C), 127.6 (2C), 128.3 (2C), 128.4 (2C), 138.6 (1C), 146.4 (1C).

MS (IFE-IT), m/z (%): 392.3 (M+1⁺,100)

[a]=+14 (c=1.9, CH₂Cl₂)

The following example compounds were prepared as described above:

### Example Compound 3

### 1-(((1S,3S)- 3-(2-(Benzyloxy)ethyl)-2,2-dimethylcyclobutyl)methyl)-4-phenylpiperidine

[a]=−19 (c=0.3, CH₂Cl₂)

### Example Compound 27

### 1-(((1R,3R)-3-(2-(Benzyloxy)ethyl)-2,2-dimethylcyclobutyl)methyl)piperidine

¹H-NMR (250 MHz, CDCl₃)

0.9 (s, 3H), 1.1 (s, 3H), 1.4 (m, 2H), 1.6 (m, 6H), 1.7 (m, 1H), 1.9 (m, 1H), 2.1 (m, 3H), 2.4 (m, 5H), 3.4 (m, 2H), 4.5 (s, 2H), 7.4 (m, 5H).

MS (IFE-IT), m/z (%): 316.3 (M+1⁺,100)

### Example Compound 2

### 1-(((1S,3S)-3-(2-(Benzyloxy)ethyl)-2,2-dimethylcyclobutyl)methyl)piperidine

[α]=−13 (c=2.0, CH₂Cl₂)

### Example Compound 31

### 4-(((1R,3R)-3-(2-(Benzyloxy)ethyl)-2,2-dimethylcyclobutyl)methyl)morpholine

¹H-NMR (250 MHz, CDCl₃)

0.9 (s, 3H), 1.1 (s, 3H), 1.6 (m, 3H), 1.9 (m, 1H), 2.0 (m, 2H), 2.1 (m, 1H), 2.5 (m, 5H), 3.4 (m, 2H), 3.7 (m, 4H), 4.5 (s, 2H), 7.4 (m, 5H).

MS (IFE-IT), m/z (%): 318.2 (M+1⁺,100)

### Example Compound 1

### 4-(((1S,3S)-3-(2-(Benzyloxy)ethyl)-2,2-dimethylcyclobutyl)methyl)morpholine

[α]=−16 (c=0.7, CH₂Cl₂)

### Example Compound 30

### 4-(((1R,3R)-3-(2-(Benzyloxy)ethyl)-2,2-dimethylcyclobutyl)methyl)thiomorpholine

¹H-NMR (250 MHz, CDCl₃)

0.9 (s, 3H), 1.1 (s, 3H), 1.5 (m, 1H), 1.7 (m, 1H), 1.9 (m, 1H), 2.0 (m, 2H), 2.2 (m, 1H), 2.4 (m, 1H), 2.7 (m, 9H), 3.4 (m, 2H), 4.5 (s, 2H), 7.4 (m, 5H).

MS (IFE-IT), m/z (%): 334.2 (M+1⁺,100)

### Example Compound 33

### 1-(((1R,3R)-3-(2-(Benzyloxy)ethyl)-2,2-dimethylcyclobutyl)methyl)pyrrolidine

¹H-NMR (250 MHz, CDCl₃)

0.9 (s, 3H), 1.1 (s, 3H), 1.6 (m, 2H), 1.8 (m, 4H), 2.1 (m, 5H), 2.4 (m, 4H), 2.6 (dd, J=11.5 Hz, 1H), 3.4 (m, 2H), 4.5 (s, 2H), 7.4 (m, 5H).

¹³C-NMR (67.5 MHz, CDCl₃)

16.5 (1C), 23.3 (2C), 30.1 (1C), 30.3 (1C), 30.8 (1C), 40.0 (1C), 40.1 (1C), 42.1 (1C), 54.5 (2C), 57.2 (1C), 69.1 (1C), 73.0 (1C), 127.0 (1C), 127.5 (2C), 128.3 (2C), 138.5 (1C).

MS (IFE-IT), m/z (%): 302.2 (M+1⁺,100)

### Example Compound 28

### 1-(((1R,3R)-3-(2-(Benzyloxy)ethyl)-2,2-dimethylcyclobutyl)methyl)-4-methylpiperidine

¹H-NMR (250 MHz, CDCl₃)

0.9 (s, 3H), 0.9 (d, J=4 Hz, 3H), 1.1 (s, 3H), 1.3 (m, 2H), 1.7 (m, 8H), 2.1 (m, 4H), 2.4 (dd, J=10.5 Hz, 1H), 2.8 (m, 2H), 3.4 (m, 2H), 4.5 (s, 2H), 7.4 (m, 5H).

¹³C-NMR (67.5 MHz, CDCl₃)

16.4 (2C), 22.0 (1C), 30.2 (1C), 30.4 (1C), 30.7 (1C), 31.2 (1C), 34.2 (1C), 34.3 (1C), 40.2 (2C), 40.5 (1C), 53.8 (1C), 54.8 (1C), 60.1 (1C), 69.2 (1C), 73.0 (1C), 127.4 (2C), 127.5 (1C), 128.3 (2C), 138.6 (1C).

MS (IFE-IT), m/z (%): 330.3 (M+1⁺,100)

### Example Compound 34

### 1-(((1R,3R)-3-(2-(Benzyloxy)ethyl)-2,2-dimethylcyclobutyl)methyl)-4-methylpiperazine

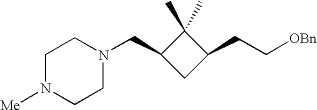

¹H-NMR (250 MHz, CDCl₃)

0.9 (s, 3H), 1.1 (s, 3H), 1.5 (m, 1H), 1.7 (m, 1H), 1.9 (m, 2H), 2.1 (m, 4H), 2.3 (s, 3H), 2.5 (m, 8H), 3.4 (m, 2H), 4.5 (s, 2H), 7.3 (m, 5H).

MS (IFE-IT), m/z (%): 331.3 (M+1⁺,100)

### Example Compound 5

### 1-(((1S,3S)-3-(2-(Benzyloxy)ethyl)-2,2-dimethylcyclobutyl)methyl)-4-methylpiperazine

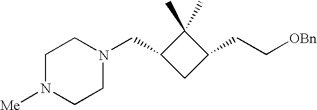

[α]=−19 (c=0.4, CH₂Cl₂)

### Example Compound 36

### 1-(((1R,3R)-3-(2-(Benzyloxy)ethyl)-2,2-dimethylcyclobutyl)methyl)-4-phenylpiperazine

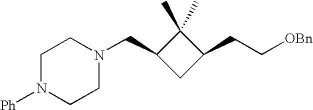

¹H-NMR (250 MHz, CDCl₃)

0.9 (s, 3H), 1.1 (s, 3H), 1.6 (m, 3H), 2.1 (m, 4H), 2.5 (m, 5H), 3.2 (m, 4H), 3.4 (m, 2H), 4.5 (s, 2H), 6.9 (m, 3H), 7.4 (m, 7H).

¹³C-NMR (67.5 MHz, CDCl₃)

16.4 (1C), 30.1 (1C), 30.4 (1C), 30.6 (1C), 40.0 (1C), 40.1 (2C), 49.1 (2C), 53.5 (2C), 59.6 (1C), 69.1 (1C), 72.9 (1C), 115.9 (2C), 119.5 (1C), 127.4 (1C), 127.6 (2C), 128.3 (2C), 129.0 (2C), 138.7 (1C), 151.4 (1C).

MS (IFE-IT), m/z (%): 393.3 (M+1⁺,100)

[α]=+11 (c=1.2, CH₂Cl₂)

### Example Compound 4

### 1-(((1S,3S)-3-(2-(Benzyloxy)ethyl)-2,2-dimethylcyclobutyl)methyl)-4-phenylpiperazine

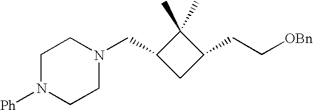

[α]=−15 (c=0.3, CH₂Cl₂)

### Example Compound 7

### 1-(((1R,3S)-3-((Benzyloxy)methyl)-2,2-dimethylcyclobutyl)methyl)piperidine

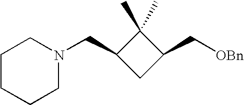

¹H-NMR (250 MHz, CDCl₃)

1.0 (s, 3H), 1.2 (s, 3H), 1.6 (m, 6H), 2.1 (m, 5H), 2.3 (m, 5H), 3.4 (m, 2H), 4.5 (d, J=2 Hz, 2H), 7.3 (m, 5H).

MS (IFE-IT), m/z (%): 302.2 (M+1⁺,100)

[α]=−8 (c=0.5, CH₂Cl₂)

### Example Compound 6

### 4-(((1R,3S)-3-((Benzyloxy)methyl)-2,2-dimethylcyclobutyl)methyl)morpholine

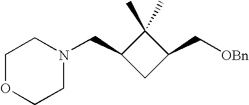

¹H-NMR (250 MHz, CDCl₃)

1.0 (s, 3H), 1.1 (s, 3H), 2.1 (m, 5H), 2.4 (m, 5H), 3.4 (m, 2H), 3.7 (m, 4H), 4.5 (d, J=2 Hz, 2H), 7.4 (m, 5H).

MS (IFE-IT), m/z (%): 304.2 (M+1⁺,100)

[α]=−3 (c=0.6, CH₂Cl₂

### Example Compound 10

### 1-(((1R,3S)-3-((Benzyloxy)methyl)-2,2-dimethylcyclobutyl)methyl)-4-methylpiperazine

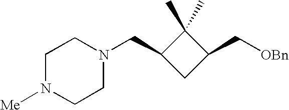

¹H-NMR (250 MHz, CDCl₃)

1.0 (s, 3H), 1.2 (s, 3H), 2.2 (m, 5H), 1.9 (m, 1H), 2.3 (s, 3H), 2.5 (m, 8H), 3.5 (m, 2H), 4.5 (d, J=2 Hz, 2H), 7.4 (m, 5H).

MS (IFE-IT), m/z (%): 317.2 (M+1⁺,100)

[α]=−3 (c=0.6, CH₂Cl₂)

### Example Compound 9

### 1-(((1R,3S)-3-((Benzyloxy)methyl)-2,2-dimethylcyclobutyl)methyl)-4-phenylpiperazine

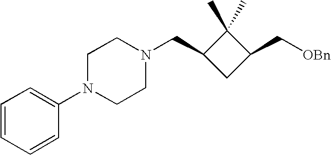

¹H-NMR (250 MHz, CDCl₃)

1.0 (s, 3H), 1.2 (s, 3H), 2.2 (m, 5H), 2.6 (m, 5H), 3.4 (m, 4H), 3.6 (m, 2H), 4.5 (d, J=2 Hz, 2H), 7.0 (m, 5H), 7.3 (m, 5H).

MS (IFE-IT), m/z (%): 379.2 (M+1⁺,100)

[α]=−9 (c=0.2, CH₂Cl₂)

### Example Compound 8

### 1-(((1R,3S)-3-((Benzyloxy)methyl)-2,2-dimethylcyclobutyl)methyl)-4-phenylpiperidine

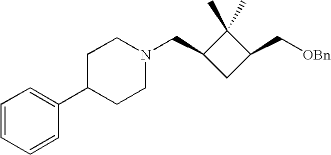

¹H-NMR (250 MHz, CDCl₃)

1.0 (s, 3H), 1.2 (s, 3H), 1.9 (m, 9H), 1.9 (m, 1H), 2.2 (m, 4H), 3.1 (m, 1H), 3.5 (m, 2H), 4.5 (d, J=2 Hz, 2H), 7.4 (m, 10H).

MS (IFE-IT), m/z (%): 378.3 (M+1⁺,100)

[α]=−18 (c=0.1, CH₂Cl₂)

### Preparation of Compounds of General Formula XII

1-(2-((1R,3R)-3-((Benzyloxy)methyl)-2,2-dimethylcyclobutyl)ethyl)piperidine

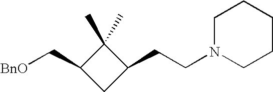

¹H-NMR (250 MHz, CDCl₃)

0.9 (s, 3H), 1.1 (s, 3H), 1.5 (m, 2H), 1.6 (m, 6H), 1.8 (m, 3H), 2.0 (m, 1H), 2.2 (m, 2H), 2.4 (m, 4H), 3.4 (m, 2H), 4.5 (s, 2H), 7.3 (m, 5H).

MS (IFE-IT), m/z (%): 316.2 (M+1⁺,100)

### 4-(2-((1R,3R)-3-((Benzyloxy)methyl)-2,2-dimethylcyclobutyl)ethyl)morpholine

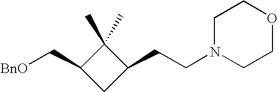

¹H-NMR (250 MHz, CDCl₃)

0.9 (s, 3H), 1.1 (s, 3H), 1.4 (m, 1H), 1.6 (m, 1H), 1.8 (m, 1H), 2.0 (m, 1H), 2.2 (m, 4H), 2.4 (m, 4H), 3.4 (m, 2H), 3.7 (m, 4H), 4.5 (s, 2H), 7.3 (m, 5H).

MS (IFE-IT), m/z (%): 318.2 (M+1⁺,100)

### Preparation of Example Compound 37

### 1-(((1R,3R)-3-(2-(benzyloxy)ethyl)-2,2-dimethylcyclobutyl)methyl)-4-phenylpiperazine oxalate

1-(((1R,3R)-3-(2-(benzyloxy)ethyl)-2,2-dimethylcyclobutyl)methyl)-4-phenylpiperazine (83 mg, 0.2 mmol) was dissolved in hot EtOH and oxalic acid dihydrate (25.8 mg, 0.2 mmol) was added. The mixture was allowed to stand at room temperature for 72 h under pentane atmosphere. The produced precipitate was filtered and dried to afford the 1-(((1R,3R)-3-(2-(benzyloxy)ethyl)-2,2-dimethylcyclobutyl)methyl)-4-phenylpiperazine oxalate (39 mg, 40% yield).

¹H-NMR (250 MHz, MeOD-d₄)

1.0 (s, 3H), 1.1 (s, 3H), 1.6 (m, 3H), 2.1 (m, 4H), 2.5 (m, 5H), 3.2 (m, 4H), 3.4 (m, 2H), 4.5 (s, 2H), 7.0 (m, 3H), 7.3 (m, 7H).

MS (IFE-IT), m/z (%): 393.3 (M+1⁺,100)

mp=162° C. (EtOH/pentane)

The following product was prepared in a similar manner:

### Example Compound 35

### 1-(((1R,3R)-3-(2-(Benzyloxy)ethyl)-2,2-dimethylcyclobutyl)methyl)-4-methylpiperazine oxalate

¹H-NMR (250 MHz, MeOD-d₄)

0.9 (s, 3H), 1.1 (s, 3H), 1.5 (m, 3H), 1.7 (m, 1H), 2.0 (m, 4H), 2.6 (s, 3H), 2.9 (m, 8H), 3.4 (m, 2H), 4.5 (s, 2H), 7.3 (m, 5H).

MS (IFE-IT), m/z (%): 331.3 (M+1⁺,100)

mp=230° C. dec. (EtOH/pentane)

A compound of general formula XIII, wherein R¹, R³ and R⁴ represent hydrogen and n is 1, can be prepared starting from (−)-α-pinene as depicted in scheme 2. below.

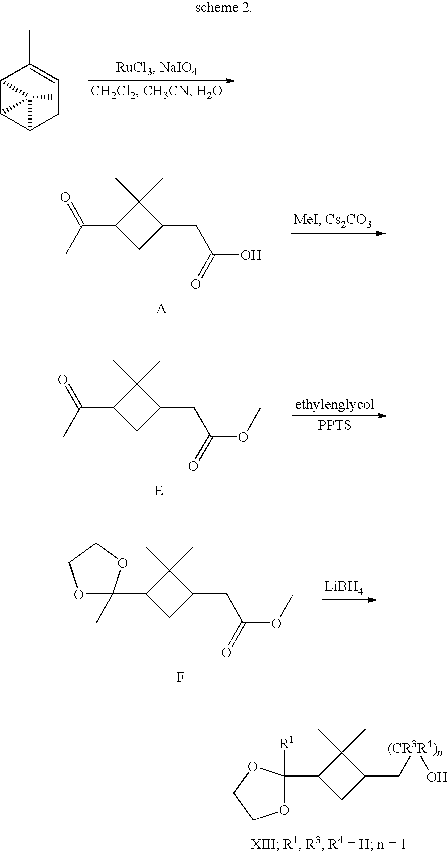

### Preparation of Compounds of General Formula XVI

### 1-((1R,3R)-2,2-Dimethyl-3-(2-morpholinoethyl)cyclobutyl)ethanone

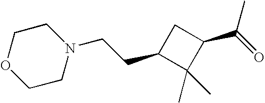

2M HCl (0.1 mL, 0.2 mmol) was added to a solution of 4-(2-((1R,3R)-2,2-dimethyl-3-(2-methyl-1,3-dioxolan-2-yl)cyclobutyl)ethyl)morpholine (200 mg, 0.7 mmol) in MeOH (10 mL) and the mixture was subsequently stirred at room temperature for 4.5 h and evaporated to dryness. The residue was poured into EtOAc and washed with saturated aqueous NaHCO₃. The organic phase was dried over MgSO₄ and the solvent was removed to afford 1-((1R,3R)-2,2-dimethyl-3-(2-morpholinoethyl)cyclobutyl)ethanone (120 mg, 71% yield) that was used in the next step without further purification.

¹H-NMR (250 MHz, CDCl₃)

0.9 (s, 3H), 1.3 (s, 3H), 1.5 (m, 2H), 1.9 (m, 3H), 2.0 (s, 3H), 2.2 (m, 2H), 2.4 (m, 4H), 2.8 (m, 1H), 3.7 (m, 4H).

The following compound of general formula XVI was prepared in a similar manner:

### 1-((1R,3R)-2,2-Dimethyl-3-(2-(piperidin-1-yl)ethyl)cyclobutyl)ethanone

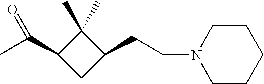

¹H-NMR (250 MHz, CDCl₃)

0.9 (s, 3H), 1.3 (s, 3H), 1.5 (m, 2H), 1.7 (m, 6H), 1.9 (m, 3H), 2.0 (s, 3H), 2.2 (m, 2H), 2.4 (m, 4H), 2.8 (m, 1H).

### Preparation of Compounds of General Formula XVII

### Example Compound 13

### 1-((1R,3R)-2,2-Dimethyl-3-(2-morpholinoethyl)cyclobutyl)-1-phenylethanol

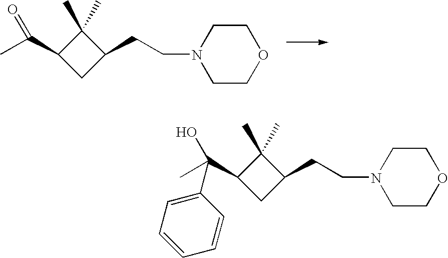

Phenyllithium [1.9 M in ether (0.8 mL, 1.5 mmol)] was added to an ice-cooled solution of 1-((1R,3R)-2,2-dimethyl-3-(2-morpholinoethyl)cyclobutyl)ethanone (170 mg, 0.7 mmol) in ahydrous THF (8 mL). The mixture was allowed to warm to room temperature and stirred overnight under nitrogen atmosphere. Excess of Phenyllithium was destroyed with wet ether and then water was added. The layers were separated and the solvent was evaporated from the organic phase. The residue was poured into EtOAc, washed with brine and dried over Mg SO4. The solvent was removed and the residue (225 mg) was chroamtographed on Baker® silica (1:3 EtOAc-hexane) to afford (S)-1-((1R,3R)-2,2-dimethyl-3-(2-morpholinoethyl)cyclobutyl)-1-phenylethanol (45 mg, 20% yield).

¹H-NMR (250 MHz, CDCl₃)

1.1 (s, 3H), 1.2 (s, 3H), 1.3 (m, 3H), 1.6 (s, 3H), 2.1 (m, 2H), 2.2 (m, 2H), 2.4 (m, 5H), 3.7 (m, 4H), 7.4 (m, 5H).

MS (IFE-IT), m/z (%): 318.3 (M+1⁺,100)

The following product was prepared in a similar manner:

### Example Compound 14

### 1-((1R,3R)-2,2-Dimethyl-3-(2-(piperidin-1-yl)ethyl)cyclobutyl)-1-phenylethanol

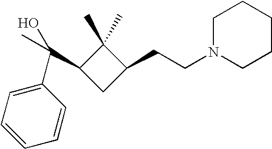

¹H-NMR (250 MHz, CDCl₃)

1.1 (s, 3H), 1.2 (s, 3H), 1.4 (m, 3H), 1.5 (m, 6H), 1.6 (s, 3H), 2.1 (m, 2H), 2.2 (m, 3H), 2.4 (m, 4H), 7.3 (m, 5H).

MS (IFE-IT), m/z (%): 316.2 (M+1⁺,100)

### Example Compound 12

### 1-((1R,3R)-2,2-Dimethyl-3-(2-(piperidin-1-yl)ethyl)cyclobutyl)ethanol

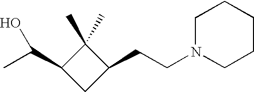

¹H-NMR (250 MHz, CDCl₃)

1.0 (s, 3H), 1.0 (d, J=6.25 Hz, 3H), 1.1 (s, 3H), 1.4 (m, 2H), 1.6 (m, 8H), 1.9 (m, 2H), 2.2 (m, 2H), 2.4 (m, 4H), 3.7 (m, 1H).

MS (IFE-IT), m/z (%): 240.1 (M+1⁺,100)

### Example Compound 11

### 1-((1R,3R)-2,2-Dimethyl-3-(2-morpholinoethyl)-cyclobutyl)ethanol)

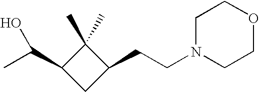

¹H-NMR (250 MHz, CDCl₃)

1.0 (s, 3H), 1.1 (d, J=6.75 Hz, 3H), 1.2 (s, 3H), 1.5 (m, 2H), 1.6 (m, 2H), 1.9 (m, 2H), 2.2 (m, 2H), 2.4 (m, 4H), 3.7 (m, 5H).

MS (IFE-IT), m/z (%): 242.1 (M+1⁺,100)

Pharmacological Data

The binding of the compounds of the present invention to sigma-1 receptors was tested as described above.

The substituted dimethylcyclobutyl compounds of general formula I show a high affinity to sigma1 receptors (table 1).

TABLE 1

| | Percent | Percent | |
|---|---|---|---|
| | Inhibition | Inhibition | |
| | Percent | Percent | |
| | Concentration | Concentration | |
| Compound | (μM) 0.1000 | (μM) 0.0100 | Binding |
| according to | SIGMA-1 | SIGMA-1 | Sigma-1 |
| example | receptor | receptor | Kᵢ (nM) |
| 26 | 50 | 23.9 | |
| 51 | 81.9 | 36.1 | |
| 48 | 72.4 | 34.3 | |
| 17 | 61.5 | 45.2 | 41.8 |
| 31 | 101.4 | 89.9 | 1.7 |
| 27 | 104.2 | 96.5 | 1.1 |
| 40 | 60.1 | 16.3 | |
| 54 | 87.1 | 42.2 | 11.7 |
| 47 | 92 | 36.8 | |
| 42 | 88 | 19.7 | |
| 25 | 98.3 | 83 | 1.2 |
| 49 | 92 | 45.7 | 4.9 |
| 53 | 73.6 | −6.6 | |
| 52 | 52.6 | −0.6 | |
| 50 | 20.9 | 2 | |
| 46 | 83 | 50.2 | 13.5 |
| 45 | 28.4 | 10.3 | |
| 44 | 82.8 | 41.7 | |
| 30 | 91.8 | 74 | 1 |
| 33 | 89.8 | 85.3 | 0.6 ± 0.2 |
| 39 | 95.8 | 94.1 | 8.4 |
| 38 | 98.5 | 88.6 | 1.2 |
| 13 | 95.2 | 77.8 | 1.2 |
| 14 | 97.5 | 75.6 | 1.9 |
| 11 | 44.4 | −8.3 | |
| 12 | 44.1 | 5.5 | |
| 28 | 100.5 | 93.5 | 5 |
| 34 | 96.2 | 92.3 | 3.6 |
| 35 | 95.9 | 76.7 | 5.3 |
| 36 | 88.3 | 65.8 | 47 |
| 29 | 95.6 | 76.9 | 16.6 |
| 32 | 99.1 | 94.6 | 3.6 |
| 37 | 98.8 | 79.7 | 33.5 |
| 19 | 42.3 | 17.1 | |
| 16 | 96.4 | 97.5 | 2.2 |
| 15 | 95.4 | 93.6 | 4.2 |
| 18 | 44.8 | −21.4 | |
| 20 | 63.1 | 31.5 | |
| 23 | 70.6 | 27.6 | |
| 24 | 92 | 69.7 | 3.3 |
| 22 | 96.8 | 91.9 | 1.4 |
| 21 | 77.1 | 25.7 | |
| 7 | 101 | 94.5 | 0.79 |
| 6 | 98.4 | 91 | 1.3 |
| 10 | 99.5 | 86.7 | 0.95 |
| 9 | 94.7 | 60 | 14.6 |
| 8 | 101.2 | 48.3 | 10.9 |
| 2 | 95.9 | 66.5 | 1.5 |
| 1 | 79.7 | 62.8 | 6.1 |
| 5 | 92.4 | 42.5 | |
| 4 | 82.2 | 24.1 | |
| 3 | 97.7 | 30.2 | |

1. A substituted dimethylcyclobutyl compound of general formula I, 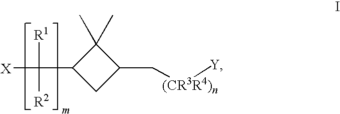
wherein
m is 1, 2, 3 or 4;
n is 0, 1, 2 or 3;
X and Y are different;
X represents a —OR⁵ moiety or a —NR⁶R⁷ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
Y represents a —OR⁸ moiety; a —NR⁹R¹⁰ moiety; a —C(═O)—OR¹¹ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R¹ represents a hydrogen atom or a linear or branched, saturated or
unsaturated, unsubstituted or at least mono-substituted aliphatic radical;
R² represents a hydrogen atom;
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, saturated or unsaturated, unsubstituted or at least mono-substituted aliphatic radical;
R⁵ and R⁸, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system
or a —C(═O)—R¹² moiety;
with the proviso that
if R⁵ represents hydrogen and m is 1,
R¹ represents a linear or branched, saturated or
unsaturated, unsubstituted or at least mono-substituted aliphatic radical;
R² represents a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system; or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R⁶, R⁷, R⁹ and R¹⁰, independently of one another, each represent a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
R¹¹ represents a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system; and
R¹² represents a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

2. A compound according to claim 1, characterized in that the stereoisomers have the general formulae Ia or Ib or Ic or Id, 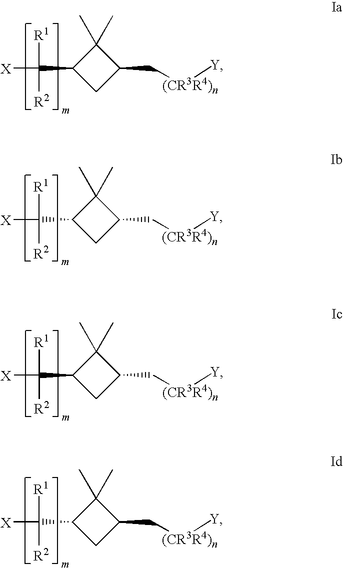
wherein
X and Y are different; and
m, X, Y, R¹, R², R³, R⁴ and n are defined as in claim 1.

3. A compound according to claim 1 or 2, characterized in that
m is 1, 2, 3 or 4;
n is 0, 1, 2 or 3;
X and Y are different;
X represents a —OR⁵ moiety or a —NR⁶R⁷ moiety;
a C₃₋₉ cycloalkyl radical or C₄₋₉ cycloalkenyl radical, which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system;
Y represents a —OR⁸ moiety; a —NR⁹R¹⁰ moiety; a —C(═O)—OR¹¹ moiety;
a C₃₋₉ cycloalkyl radical or C₄₋₉ cycloalkenyl radical, which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system;
R¹ represents a hydrogen atom or a linear or branched, unsubstituted or at least mono-substituted C₁₋₁₀ alkyl radical, C₂₋₁₀ alkenyl radical or C₂₋₁₀ alkinyl radical;
R² represents a hydrogen atom;
a linear or branched, unsubstituted or at least mono-substituted C₁₋₁₀ alkyl radical, C₂₋₁₀ alkenyl radical or C₂₋₁₀ alkinyl radical;
a C₃₋₉ cycloalkyl radical or C₄₋₉ cycloalkenyl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, unsubstituted or at least mono-substituted C₁₋₁₀ alkyl radical, C₂₋₁₀ alkenyl radical or C₂₋₁₀ alkinyl radical;
R⁵ and R⁸, independently of one another, each represent a hydrogen atom or a linear or branched, unsubstituted or at least mono-substituted C₁₋₁₀ alkyl radical, C₂₋₁₀ alkenyl radical or C₂₋₁₀alkinyl radical;
a C₃₋₉ cycloalkyl radical or C₄₋₉ cycloalkenyl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated, unsaturated or aromatic mono- or bicyclic ring system;
an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system
or a —C(═O)—R¹² moiety;
R⁶, R⁷, R⁹ and R¹⁰, independently of one another, each represent a linear or branched, unsubstituted or at least mono-substituted C₁₋₁₀ alkyl radical, C₂₋₁₀ alkenyl radical or C₂₋₁₀ alkinyl radical;
a C₃₋₉ cycloalkyl radical or C₄₋₉ cycloalkenyl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic 3-, 4-, 5-, 6-, 7-, 8-, 9-, 10-, 11-, 12-, 13- or 14-membered heterocyclic ring which may contain 1, 2 or 3 additional heteroatom(s) independently selected from the group consisting of nitrogen, oxygen and sulfur as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic 3-, 4-, 5-, 6-, 7-, 8-, 9-, 10-, 11-, 12-, 13- or 14-membered heterocyclic ring which may contain 1, 2 or 3 additional heteroatom(s) independently selected from the group consisting of nitrogen, oxygen and sulfur as ring member(s) and which may be condensed with an unsubstituted or at least mono-substituted saturated, unsaturated or aromatic mono- or bicyclic ring system;
R¹¹ represents a linear or branched, unsubstituted or at least mono-substituted C₁₋₁₀ alkyl radical, C₂₋₁₀ alkenyl radical or C₂₋₁₀ alkinyl radical;
a C₃₋₉ cycloalkyl radical or C₄₋₉ cycloalkenyl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system; and
R¹² represents a hydrogen atom or a linear or branched, unsubstituted or at least mono-substituted C₁₋₁₀ alkyl radical, C₂₋₁₀ alkenyl radical or C₂₋₁₀ alkinyl radical;
a C₃₋₉ cycloalkyl radical or C₄₋₉ cycloalkenyl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated, unsaturated or aromatic mono- or bicyclic ring system;
or an unsubstituted or at least mono-substituted 6-, 10- or 14-membered aryl or a 5-, 6-, 8-, 9-, 10-, 11-, 12-, 13- or 14-membered mono-, bi- or tricyclic heteroaryl radical, which may be bonded via a linear or branched, unsubstituted or at least mono-substituted C₁₋₆ alkylene, C₂₋₆ alkenylene or C₂₋₆ alkinylene group and which may be condensed with an unsubstituted or at least mono-substituted saturated or unsaturated, but not aromatic, mono- or bicyclic ring system;
whereby
the aforementioned C₁₋₁₀ alkyl radical, C₂₋₁₀ alkenyl radical or C₂₋₁₀ alkinyl radicals may in each case be unsubstituted or optionally substituted with 1, 2, 3, 4 or 5 substituent(s) independently selected from the group consisting of F, Cl, Br, I, —CN, —CF₃, —OCF₃, —SCF₃, —OH, —SH, —NH₂, —O—C₁₋₅-alkyl, —S—C₁₋₅-alkyl, —NH(C₁₋₅-alkyl) and —N(C₁₋₅-alkyl)₂;
the aforementioned C₃₋₉ cycloalkyl radicals and C₄₋₉ cycloalkenyl radicals may in each case be unsubstituted or optionally substituted with 1, 2, 3, 4 or 5 substituent(s) independently selected from the group consisting of oxo (═O), thioxo (═S), C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenethyl, phenoxy and benzyl, whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
the aforementioned 3-, 4-, 5-, 6-, 7-, 8-, 9-, 10-, 11-, 12-, 13- or 14-membered heterocyclic rings may in each case be unsubstituted or optionally substituted with 1, 2, 3, 4 or 5 substituent(s) independently selected from the group consisting of oxo (═O), thioxo (═S), C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenethyl, phenoxy and benzyl, whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
the aforementioned C₃₋₉ cycloalkyl radicals and C₄₋₉ cycloalkenyl radicals in each case may optionally contain 1, 2 or 3 heteroatom(s) independently selected from the group consisting of nitrogen, oxygen and sulfur as ring member(s);
the aforementioned C₁₋₆ alkylene, C₂₋₆ alkenylene or C₂₋₆ alkinylene groups may in each case be unsubstituted or substituted with 1, 2 or 3 substituent(s) independently selected from the group consisting of F, Cl, Br, I, —CN, —CF₃, —OCF₃, —SCF₃, —OH, —SH, —NH₂, —O—C₁₋₅-alkyl, —S—C₁₋₅-alkyl, —NH(C₁₋₅-alkyl) and —N(C₁₋₅-alkyl)₂;
the rings of the aforementioned ring system are in each case independently of one another 5-, 6- or 7-membered and may in each case independently of one another optionally contain 1, 2 or 3 heteroatom(s) independently selected from the group consisting of nitrogen, oxygen and sulfur;
and the rings of the ring system may in each case be unsubstituted or substituted with 1, 2 or 3 substituent(s) independently selected from the group consisting of oxo (═O), thioxo (═S), C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenethyl, phenoxy and benzyl, whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂; the 6-, 10- or 14-membered aryl or 5-, 6-, 8-, 9-, 10-, 11-, 12-, 13- or 14-membered mono-, bi- or tricyclic heteroaryl radicals may be unsubstituted or optionally substituted with 1, 2, 3, 4 or 5 substituent(s) independently selected from the group consisting of C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenethyl, phenoxy and benzyl, whereby in each occurrence C₁₋₅-alkyl may be linear or branched and whereby said cyclic substituent(s) may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
and the aforementioned 5-, 6-, 8-, 9-, 10-, 11-, 12-, 13- or 14-membered heteroaryl radicals in each case optionally contain 1, 2, 3 or 4 heteroatom(s) independently selected from the group consisting of nitrogen, oxygen and sulfur as ring member(s);
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

4. A compound according to one or more of claims 1 to 3, characterized in that n is 0 or 1.

5. A compound according to one or more of claims 1 to 4, characterized in that m is 1.

6. A compound according one or more of claims 1 to 5, characterized in that
X and Y are different; and
X represents a —OR⁵ moiety or a —NR⁶R⁷ moiety; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl, azepanyl (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S-OH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

7. A compound according one or more of claims 1 to 6, characterized in that
X and Y are different; and
Y represents a —OR⁸ moiety; a —NR⁹R¹⁰ moiety; a —C(═O)—OR¹¹ moiety; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S-OH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

8. A compound according to one or more of claims 1 to 7, characterized in that
R¹ represents a hydrogen atom or a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅.

9. A compound according to one or more of claims 1 to 8, characterized in that
R² represents a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(OH₃)₃, —S—CH₃, —S—C₂H₅, —S—CH₂—CH₂—CH₃, —S—CH(CH₃)₂, —S—C(OH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(OH₃)₂, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

10. A compound according to one or more of claims 1 to 9, characterized in that R³ and R⁴ both represent a hydrogen atom.

11. A compound according to one or more of claims 1 to 10, characterized in that
R⁵ and R⁸, independently of one another, each represent a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of imidazolidinyl, aziridinyl, azetidinyl, pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl; which may be bonded via a —(CH₂)_{1, 2 or 3}-group and which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃
or a —C(═O)—R¹² moiety;
with the proviso that
if R⁵ represents hydrogen and m is 1,
R¹ represents a linear or branched, saturated or
unsaturated, unsubstituted or at least mono-substituted aliphatic radical;
R² represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system; or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system.

12. A compound according to one or more of claims 1 to 11, characterized in that
R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be bonded via a —(CH₂)_{1, 2 or 3}-group and which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(OH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

13. A compound according to one or more of claims 1 to 12, characterized in that
R⁶ and R⁷ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)——C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl, phenoxy and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

14. A compound according to one or more of claims 1 to 13, characterized in that
R⁹ and R¹⁰ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl, phenoxy and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

15. A compound according to one or more of claims 1 to 14, characterized in that
R¹¹ represents a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—N H₂, —CH₂—N H—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(OH₃)₂, —O—C(OH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; and
R¹² represents a hydrogen atom; a radical selected from the group consisting of methyl, ethyl, n-propyl, isopropyl, n-butyl, sec-butyl, isobutyl, tert-butyl, n-pentyl, neo-pentyl, n-hexyl, vinyl, allyl, ethinyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH, —CH₂—CH₂—SH, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; a (hetero)cycloaliphatic radical selected from the group consisting of cyclopropyl, cyclobutyl, cyclopentyl, cyclohexyl, cycloheptyl, cyclooctyl, cyclononyl, imidazolidinyl, aziridinyl, azetidinyl, pyrrolidinyl, piperidinyl, morpholinyl, thiomorpholinyl, piperazinyl, pyrazolidinyl and azepanyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(C H₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

16. A compound according to one or more of claims 1 to 15, characterized in that
m is 1 or 2;
n is 0 or 1;
X and Y are different;
X represents a —OR⁵ moiety or a —NR⁶R⁷ moiety; or a (hetero)cycloaliphatic radical selected from the group consisting of pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH and —NH₂;
Y represents a —OR⁸ moiety; a —NR⁹R¹⁰ moiety; a —C(═O)—OR¹¹ moiety; or a (hetero)cycloaliphatic radical selected from the group consisting of pyrrolidinyl, piperidinyl, morpholinyl, thiomorpholinyl, piperazinyl, pyrazolidinyl, azepanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl and octahydro-cyclopenta[c]pyrrolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH and —NH₂;
R¹ represents a hydrogen atom or a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH;
R² represents a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl and pyridinyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅—S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H and —CFH₂;
R³ and R⁴ both represent a hydrogen atom;
R⁵ and R⁸, independently of one another, each represent a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH and —CH₂—SH; an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H, —OF H₂ and —S(═O)₂—CH₃
or a —C(═O)—R¹² moiety;
R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, oxazolyl, isoxazolyl, thiazolyl, isothiazolyl, imidazolyl, pyrazolyl, oxadiazolyl, thiadiazolyl, triazolyl, pyridinyl, pyridazinyl, pyrimidinyl, pyrazinyl, quinolinyl, isoquinolinyl, benzo[b]furanyl, benzo[b]thiophenyl, benzothiadiazolyl and imidazo[2,1-b]thiazolyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;
or R⁶ and R⁷ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of oxo (═O), thioxo (═S), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃; phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
or R⁹ and R¹⁰ together with the bridging nitrogen atom form a heterocyclic ring selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, indolinyl, isoindolinyl, (1,2,3,4)-tetrahydroquinolinyl, (1,2,3,4)-tetrahydroisoquinolinyl, octahydro-cyclopenta[c]pyrrolyl, decahydroquinolinyl, dodecahydrocarbazolyl, 9H-carbazolyl, decahydroisoquinolinyl, (2,3)-dihydro-1H-cyclopenta[b]indolyl, [1,2,3,4]-tetrahydroquinazolinyl, [3,4]-dihydro-2H-benzo[1,4]oxazinyl, (4,5,6,7)-tetrahydro-1H-indolyl, pyrrolyl, imidazolyl, pyrazolyl and triazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
R¹¹ represents a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, and pyridinyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —S—CH₃, —S—C₂H₅, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃; and
R¹² represents a hydrogen atom; a radical selected from the group consisting of methyl, ethyl, n-propyl, isopropyl, n-butyl, sec-butyl, isobutyl, tert-butyl, n-pentyl, —CF₃, —CFH₂, —CF₂H, —CH₂—CF₃, —CF₂—CF₃, —CH₂—CN, —CH₂—CH₂—CN, —CH₂—OH, —CH₂—CH₂—OH, —CH₂—SH and —CH₂—CH₂—SH; or an aryl or heteroaryl radical selected from the group consisting of phenyl, naphthyl, furyl (furanyl), thienyl (thiophenyl), pyrrolyl, and pyridinyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, —S—CH₃, —S—C₂H₅, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

17. A compound according to one or more of claims 1 to 16, characterized in that
m is 1;
n is 0 or 1;
X and Y are different;
X represents a —OR⁵ moiety or a —NR⁶R⁷ moiety;
Y represents a —OR⁸ moiety; a —NR⁹R¹⁰ moiety or a —C(═O)—OR¹¹ moiety;
R¹ represents a hydrogen atom or a radical selected from the group consisting of methyl, ethyl, n-propyl, isopropyl, n-butyl, sec-butyl, isobutyl, tert-butyl and n-pentyl;
R² represents a hydrogen atom; a radical selected from the group consisting of methyl, ethyl, n-propyl, isopropyl, n-butyl, sec-butyl, isobutyl, tert-butyl and n-pentyl; or an aryl radical selected from the group consisting of phenyl and naphthyl, which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅—S—CH₃, —S—C₂H₅, —C(═O)—OH, —C(═O)—O—CH₃, —C(═O)—O—C₂H₅, —C(═O)—CH₃, —C(═O)—C₂H₅, F, Cl, Br, I, —CN, —CF₃, —OCF₃, —SCF₃, —OH, —SH, —NH₂, —NO₂, —CHO, —CF₂H and —CFH₂;
R³ and R⁴ both represent a hydrogen atom;
R⁵ and R⁸, independently of one another, each represent a hydrogen atom; a radical selected from the group consisting of methyl, ethyl, n-propyl, isopropyl, n-butyl, sec-butyl, isobutyl, tert-butyl and n-pentyl; an aryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅—F, Cl, Br, I, —CN, —CF₃, —OCF₃, —SCF₃, —OH, —SH, —NH₂, —NO₂, —CHO, —CF₂H, —CF H₂ and —S(═O)₂—CH₃
or a —C(═O)—R¹² moiety;
R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;
or R⁶ and R⁷ together with the bridging nitrogen atom form a moiety selected from the group consisting of 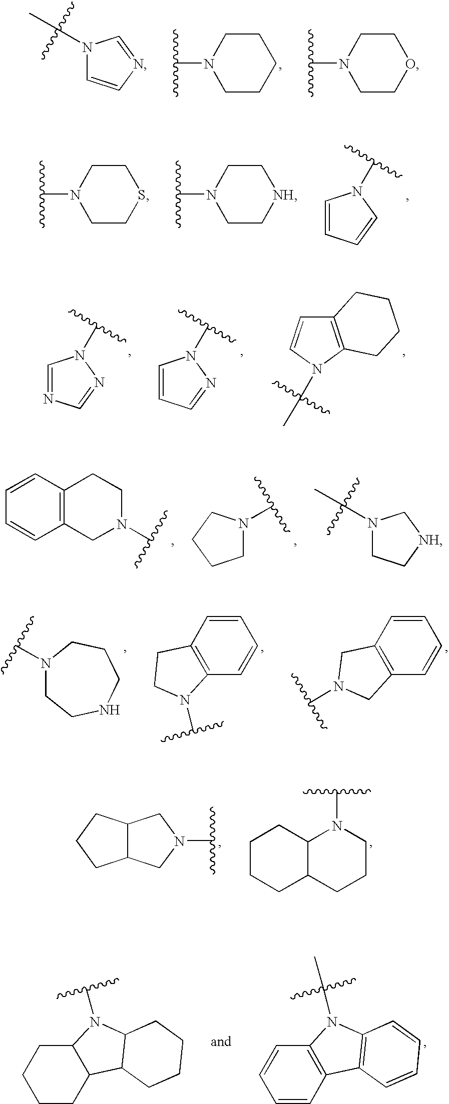
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
or R⁹ and R¹⁰ together with the bridging nitrogen atom form a moiety selected from the group consisting of 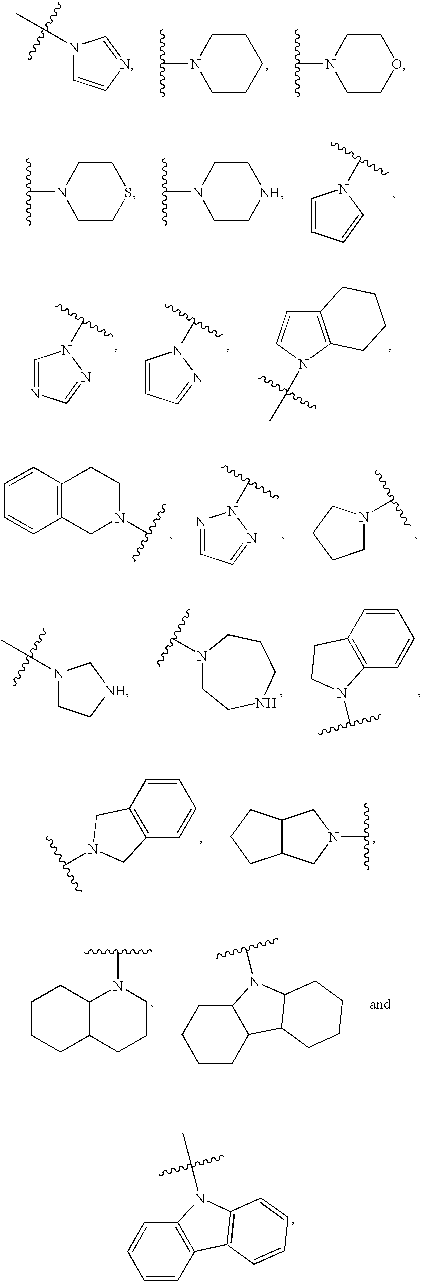
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
R¹¹ represent a radical selected from the group consisting of methyl, ethyl, n-propyl, isopropyl, n-butyl, sec-butyl, isobutyl, tert-butyl and n-pentyl; and
R¹² represent a hydrogen atom; or a radical selected from the group consisting of methyl, ethyl, n-propyl, isopropyl, n-butyl, sec-butyl, isobutyl, tert-butyl and n-pentyl;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

18. A compound according to one or more of claims 1 to 17, characterized in that
m is 1;
n is 0 or 1;
X and Y are different;
X represents a —OR⁵ moiety or a —NR⁶R⁷ moiety;
Y represents a —OR⁸ moiety; a —NR⁹R¹⁰ moiety or a —C(═O)—OR¹¹ moiety;
R¹ represents a hydrogen atom or a radical selected from the group consisting of methyl, ethyl, n-propyl, isopropyl, n-butyl, sec-butyl, isobutyl, tert-butyl and n-pentyl;
R² represents a hydrogen atom; a radical selected from the group consisting of methyl, ethyl, n-propyl, isopropyl, n-butyl, sec-butyl, isobutyl, tert-butyl and n-pentyl; or a phenyl radical which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, F, Cl, Br, I, —CN, —CF₃, —OCF₃ and —SCF₃;
R³ and R⁴ both represent a hydrogen atom;
R⁵ and R⁸, independently of one another, each represent a hydrogen atom; a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅, F, Cl, Br, I, —CN, —CF₃ and —OCF₃
or a —C(═O)—R¹² moiety;
R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, F, Cl, Br, I, —CN, —CF₃, —OCF₃ and —SCF₃;
or R⁶ and R⁷ together with the bridging nitrogen atom form a moiety selected from the group consisting of 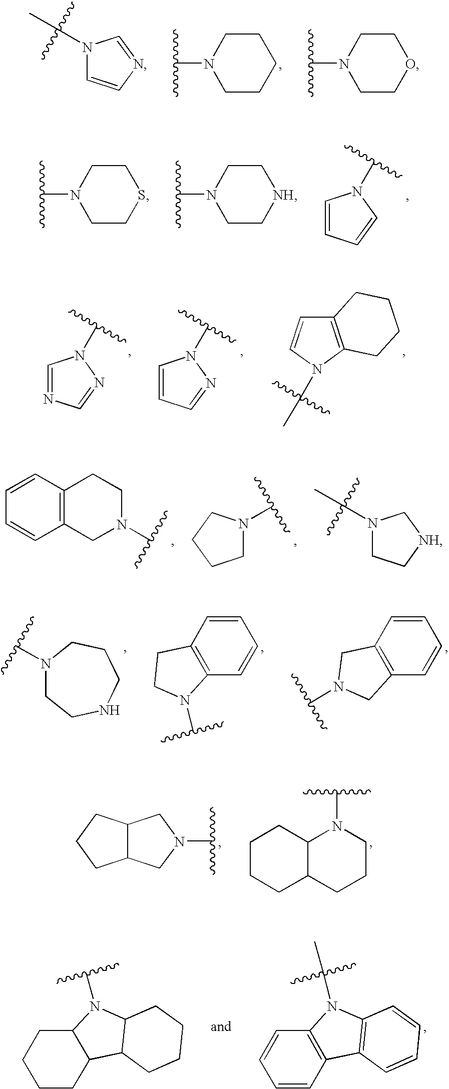
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
or R⁹ and R¹⁰ together with the bridging nitrogen atom form a moiety selected from the group consisting of 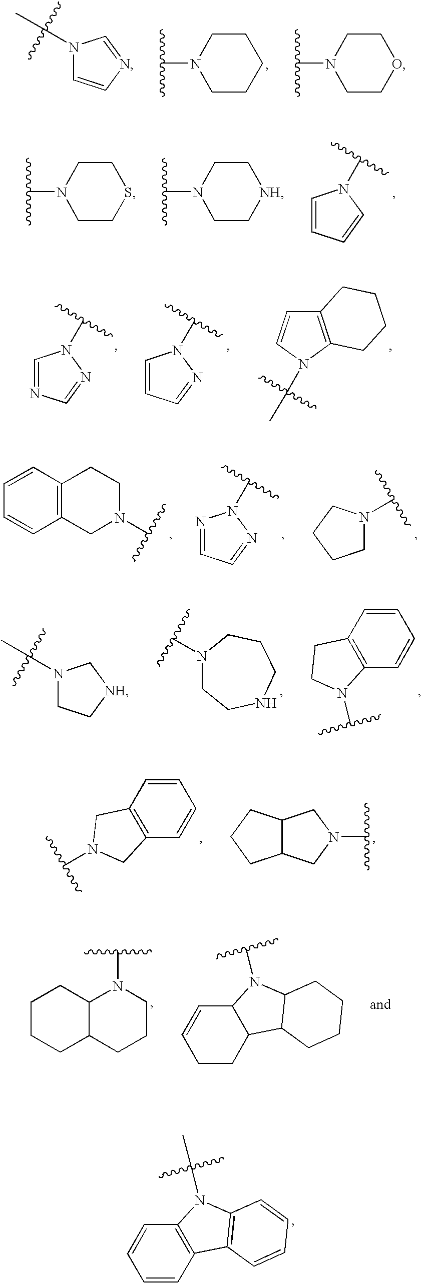
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
and R¹¹ and R¹², independently of one another, represents a radical selected from the group consisting of methyl, ethyl, n-propyl, isopropyl, n-butyl, sec-butyl, isobutyl, tert-butyl and n-pentyl;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

19. A compound according to one or more of claims 1 to 18, characterized in that
m is 1;
n is 0 or 1;
X and Y are different;
X represents a —OR⁵ moiety or a —NR⁶R⁷ moiety;
Y represents a —OR⁸ moiety; a —NR⁹R¹⁰ moiety or a —C(═O)—OR¹¹ moiety;
R¹ represents a hydrogen atom or a radical selected from the group consisting of methyl, ethyl, n-propyl, isopropyl, n-butyl, sec-butyl, isobutyl, tert-butyl and n-pentyl;
R² represents a hydrogen atom; a radical selected from the group consisting of methyl, ethyl, n-propyl, isopropyl, n-butyl, sec-butyl, isobutyl, tert-butyl and n-pentyl; or a phenyl radical which may be unsubstituted or substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, F, Cl, Br, I, —CN, —CF₃, —OCF₃ and —SCF₃;
R³ and R⁴ both represent a hydrogen atom;
R⁵ and R⁸, independently of one another, each represent a hydrogen atom; or a phenyl radical, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅, F, Cl, Br, I, —CN, —CF₃ and —OCF₃ or a —C(═O)—R¹² moiety;
R⁶, R⁷, R⁹ and R¹⁰, independently of one another, each represent a radical selected from the group consisting of methyl, ethyl, n-propyl, isopropyl, n-butyl, sec-butyl, isobutyl, tert-butyl and n-pentyl; or a phenyl radical, which is bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, F, Cl, Br, I, —CN, —CF₃, —OCF₃ and —SCF₃;
or R⁶ and R⁷ together with the bridging nitrogen atom form a moiety selected from the group consisting of 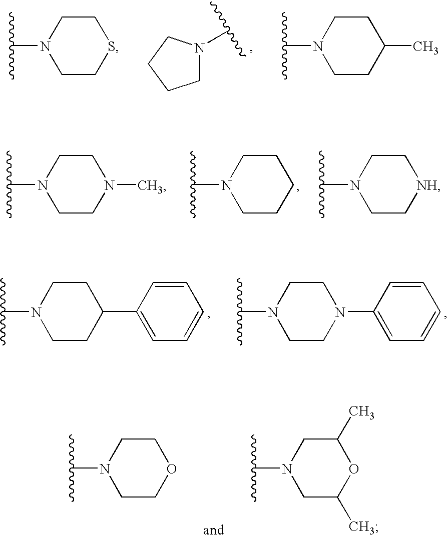
or R⁹ and R¹⁰ together with the bridging nitrogen atom form a moiety selected from the group consisting of 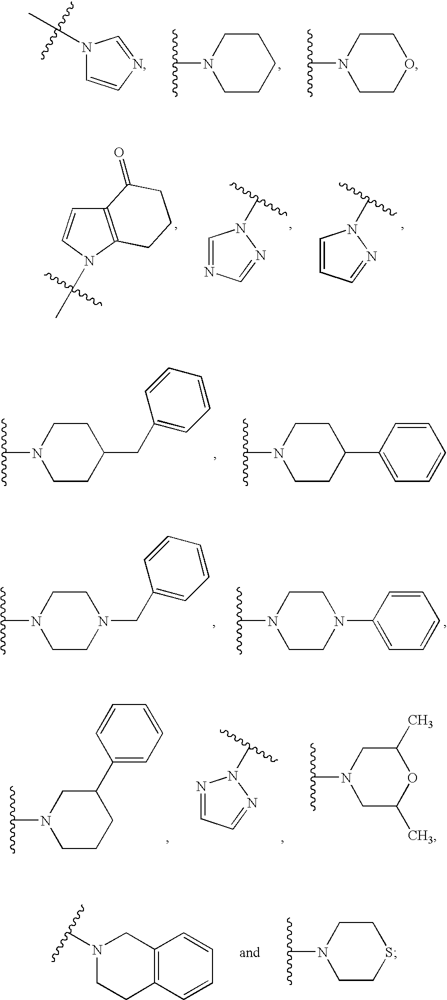
and R¹¹ and R¹², independently of one another, represent a radical selected from the group consisting of methyl, ethyl, n-propyl, isopropyl, n-butyl, sec-butyl, isobutyl, tert-butyl and n-pentyl;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

20. A compound of general formula Ie according to one or more of claims 1 to 17, 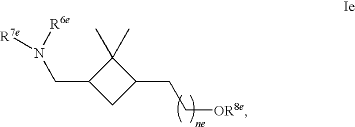
wherein
ne is 0 or 1;
R^{6e} and R^{7e}, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;
or R^{6e} and R^{7e} together with the bridging nitrogen atom form a moiety selected from the group consisting of 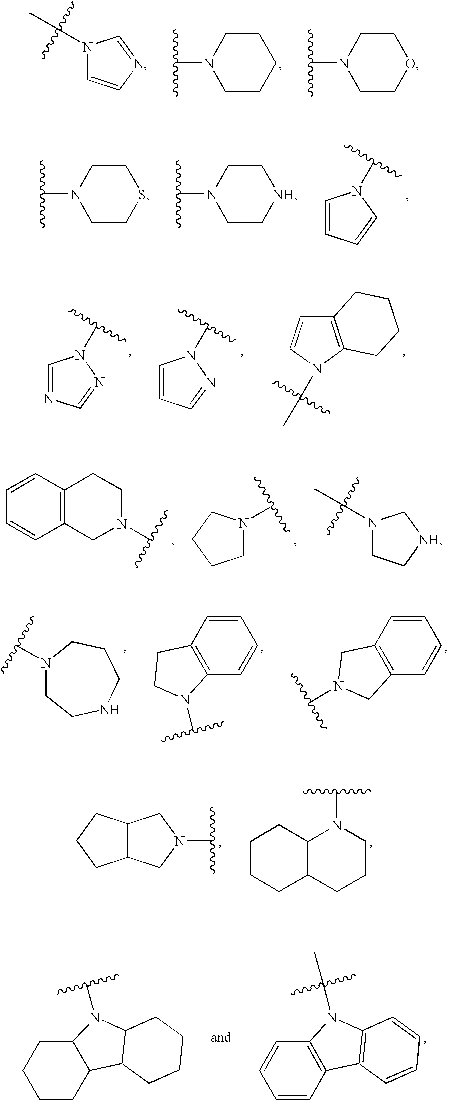
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
R^{8e} represents a hydrogen atom; a radical selected from the group consisting of methyl, ethyl, n-propyl, isopropyl, n-butyl, sec-butyl, isobutyl, tert-butyl and n-pentyl; an aryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, —O—CH₃, —O—C₂H₅—F, Cl, Br, I, —CN, —CF₃, —OCF₃, —SCF₃, —OH, —SH, —NH₂, —NO₂, —CHO, —CF₂H, —CF H₂ and —S(═O)₂—CH₃ or a —C(═O)—R^{12e} moiety;
and R^{12e} represents a radical selected from the group consisting of methyl, ethyl, n-propyl, isopropyl, n-butyl, sec-butyl, isobutyl, tert-butyl and n-pentyl;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

21. A compound according to claim 20, characterized in that the stereoisomers have the general formulae If or Ig or Ih or Ij, 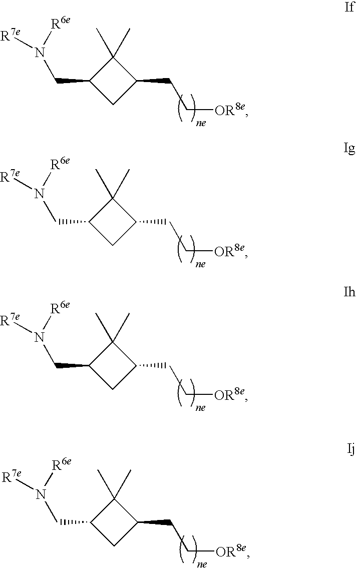
wherein ne, R^{6e}, R^{7e} and R^{8e} are defined as in claim 19.

22. A compound of general formula Ik according to one or more of claims 1 to 17, 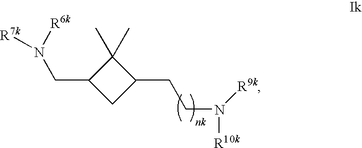
wherein
R^{6k}R^{7k}N— and R^{9k}R^{10k}N— are different;
R^{6k}, R^{7k}, R^{9k} and R^{10k}, independently of one another, each represent a radical selected from the group consisting of methyl, ethyl, n-propyl, isopropyl, n-butyl, sec-butyl, isobutyl, tert-butyl and n-pentyl; or an aryl or heteroaryl radical selected from the group consisting of phenyl and naphthyl, which may be bonded via a —(CH₂)_{1, 2 or 3}-group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, —O—CH₃, —O—C₂H₅, —S—CH₃, —S—C₂H₅, F, Cl, Br, I, —CN, —CF₃, —OCF₃, —SCF₃, —OH, —SH, —NH₂—NO₂, —CHO, —CF₂H, —CFH₂, —C(═O)—NH₂ and —S(═O)₂—CH₃;
or R^{6k} and R^{7k} together with the bridging nitrogen atom form a moiety selected from the group consisting of 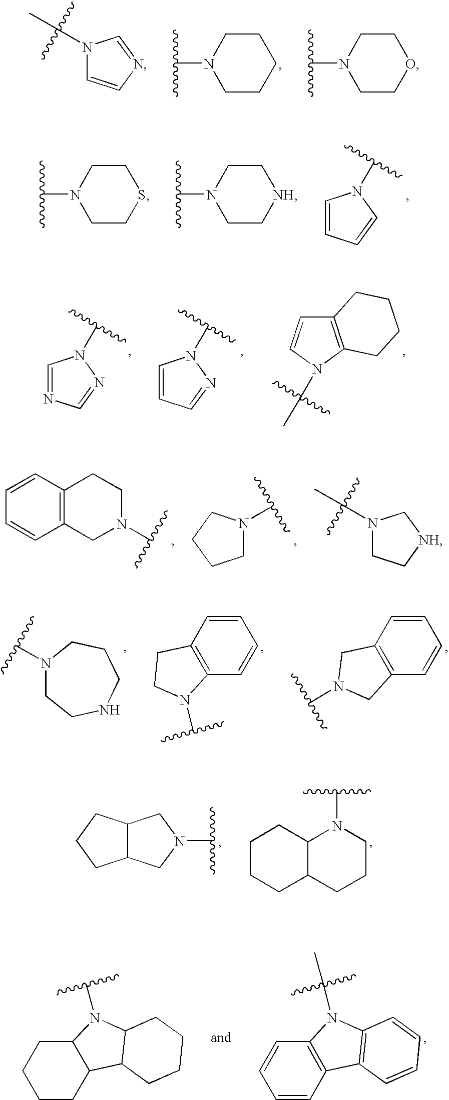
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(═O)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
or R^{9k} and R^{10k} together with the bridging nitrogen atom form a moiety selected from the group consisting of 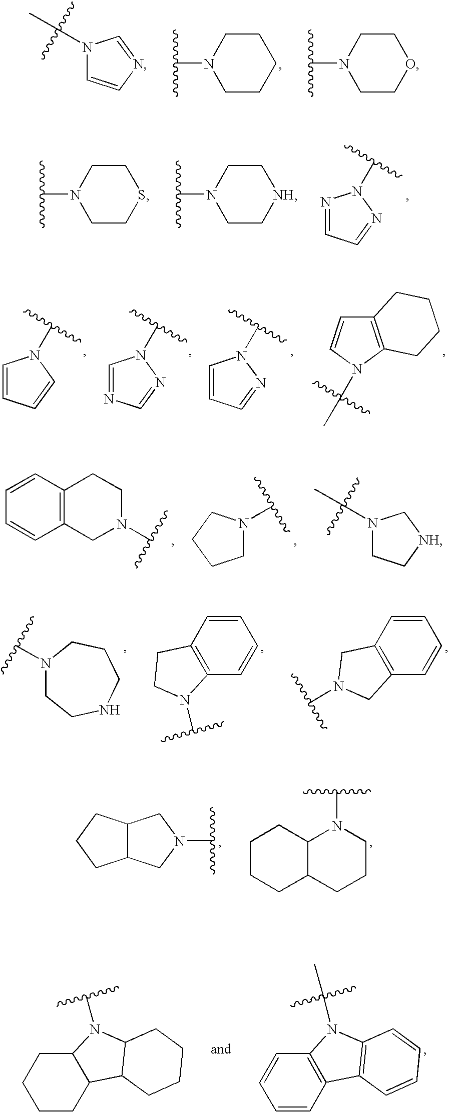
which may be unsubstituted or optionally substituted in any position including the —NH-groups with 1, 2, 3, 4 or 5 substituent(s) independently selected from the group consisting of oxo (═O), methyl, ethyl, n-propyl, isopropyl, n-butyl, tert-butyl, sec-butyl, —O—CH₃, —O—C₂H₅, —S—CH₃, —S—C₂H₅, —C(═O)—OH, —C(═O)—O—CH₃, —C(═O)—O—C₂H₅, —C(═O)—O—CH₂—CH₂—CH₃, —C(═O)—O—CH(CH₃)₂, —C(═O)—O—C(CH₃)₃, —C(═O)—CH₃, —C(═O)—C₂H₅, —C(αO)—CH₂—CH₂—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, phenyl, phenethyl and benzyl, whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

23. A compound according to claim 22, characterized in that the stereoisomers have the general formulae Im or In or Io or Ip, 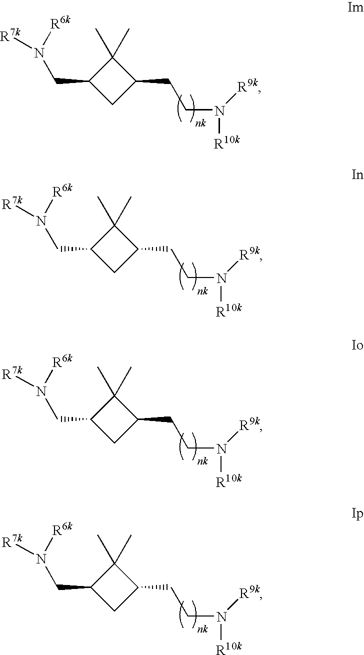
wherein nk, R^{6k}, R^{7k}, R^{9k} and R^{10k} are defined as in claim 21.

24. A compound according to one or more of claims 1 to 23 selected from the group consisting of
[1] 4-(((1S,3S)-3-(2-(benzyloxy)ethyl)-2,2-dimethylcyclobutyl)methyl)-morpholine
[2] 1-(((1S,3S)-3-(2-(benzyloxy)ethyl)-2,2-dimethylcyclobutyl)methyl)-piperidine
[3] 1-(((1S,3S)-3-(2-(benzyloxy)ethyl)-2,2-dimethylcyclobutyl)methyl)-4-phenylpiperidine
[4] 1-(((1S,3S)-3-(2-(benzyloxy)ethyl)-2,2-dimethylcyclobutyl)methyl)-4-phenylpiperazine
[5] 1-(((1S,3S)-3-(2-(benzyloxy)ethyl)-2,2-dimethylcyclobutyl)methyl)-4-methylpiperazine
[6] 4-(((1R,3S)-3-(benzyloxymethyl)-2,2-dimethylcyclobutyl)methyl)-morpholine
[7] 1-(((1R,3S)-3-(benzyloxymethyl)-2,2-dimethylcyclobutyl)methyl)-piperidine
[8] 1-(((1R,3S)-3-(benzyloxymethyl)-2,2-dimethylcyclobutyl)methyl)-4-phenylpiperidine
[9] 1-(((1R,3S)-3-(benzyloxymethyl)-2,2-dimethylcyclobutyl)methyl)-4-phenylpiperazine
[10] 1-(((1R,3S)-3-(benzyloxymethyl)-2,2-dimethylcyclobutyl)methyl)-4-methylpiperazine
[13] 1-((1R,3R)-2,2-dimethyl-3-(2-morpholinoethyl)cyclobutyl)-1-phenylethanol
[14] 1-((1R,3R)-2,2-dimethyl-3-(2-(piperidin-1-yl)ethyl)cyclobutyl)-1-phenylethanol
[15] 4-(((1R,3R)-2,2-dimethyl-3-(2-phenoxyethyl)cyclobutyl)methyl)-morpholine
[16] 1-(((1R,3R)-2,2-dimethyl-3-(2-phenoxyethyl)cyclobutyl)methyl)piperidine
[17] 2-((1R,3R)-2,2-dimethyl-3-(piperidin-1-ylmethyl)cyclobutyl)ethanol
[18] 2-((1S,3S)-2,2-dimethyl-3-(piperidin-1-ylmethyl)cyclobutyl)ethanol
[19] 2-((1 R,3R)-2,2-dimethyl-3-(morpholinomethyl)cyclobutyl)ethanol
[20] 2-((1 S,3S)-2,2-dimethyl-3-(morpholinomethyl)cyclobutyl)ethanol
[21] 2-((1R,3R)-2,2-dimethyl-3-((4-methylpiperidin-1-yl)methyl)cyclobutyl)-ethanol
[22] 2-((1R,3R)-2,2-dimethyl-3-((4-phenylpiperidin-1-yl)methyl)cyclobutyl)-ethanol
[23] 2-((1R,3R)-3-(((2S,6R)-2,6-dimethylmorpholino)methyl)-2,2-dimethylcyclobutyl)ethanol
[24] 2-((1R,3R)-3-(N-benzyl-N-methylamino)methyl)-2,2-dimethylcyclobutyl)-ethanol
[26] methyl 2-((1R,3R)-2,2-dimethyl-3-(piperidin-1-ylmethyl)cyclobutyl)-acetate
[27] 1-(((1R,3R)-3-(2-(benzyloxy)ethyl)-2,2-dimethylcyclobutyl)methyl)-piperidine
[28] 1-(((1R,3R)-3-(2-(benzyloxy)ethyl)-2,2-dimethylcyclobutyl)methyl)-4-methylpiperidine
[29] 1-(((1R,3R)-3-(2-(benzyloxy)ethyl)-2,2-dimethylcyclobutyl)methyl)-4-phenylpiperidine
[30] 4-(((1R,3R)-3-(2-(benzyloxy)ethyl)-2,2-dimethylcyclobutyl)methyl)-thiomorpholine
[31] 4-(((1R,3R)-3-(2-(benzyloxy)ethyl)-2,2-dimethylcyclobutyl)methyl)-morpholine
[32] (2S,6R)-4-(((1R,3R)-3-(2-(benzyloxy)ethyl)-2,2-dimethylcyclobutyl)-methyl)-2,6-dimethylmorpholine
[33] 1-(((1R,3R)-3-(2-(benzyloxy)ethyl)-2,2-dimethylcyclobutyl)methyl)-pyrrolidine
[34] 1-(((1R,3R)-3-(2-(benzyloxy)ethyl)-2,2-dimethylcyclobutyl)methyl)-4-methylpiperazine
[35] 1-(((1R,3R)-3-(2-(benzyloxy)ethyl)-2,2-dimethylcyclobutyl)methyl)-4-methylpiperazine oxalate
[36] 1-(((1R,3R)-3-(2-(benzyloxy)ethyl)-2,2-dimethylcyclobutyl)methyl)-4-phenylpiperazine
[37] 1-(((1R,3R)-3-(2-(benzyloxy)ethyl)-2,2-dimethylcyclobutyl)methyl)-4-phenylpiperazine oxalate
[38] 1-(2-((1R,3R)-3-(benzyloxymethyl)-2,2-dimethylcyclobutyl)ethyl)-piperidine
[39] 4-(2-((1R,3R)-3-(benzyloxymethyl)-2,2-dimethylcyclobutyl)ethyl)-morpholine
[40] 4-(((1R,3R)-2,2-dimethyl-3-(2-(3-phenylpiperidin-1-yl)ethyl)cyclobutyl)-methyl)-morpholine
[41] 4-(((1R,3R)-2,2-dimethyl-3-(2-(4-phenylpiperidin-1-yl)ethyl)cyclobutyl)-methyl)morpholine
[42] 4-(((1R,3R)-3-(2-(4-benzylpiperidin-1-yl)ethyl)-2,2-dimethylcyclobutyl)-methyl)morpholine
[43] 4-(((1R,3R)-3-(2-(1H-imidazol-1-yl)ethyl)-2,2-dimethylcyclobutyl)-methyl)morpholine
[44] 4-(((1R,3R)-3-(2-(1H-pyrazol-1-yl)ethyl)-2,2-dimethylcyclobutyl)-methyl)morpholine
[45] 4-(((1R,3R)-3-(2-(1H-1,2,4-triazol-1-yl)ethyl)-2,2-dimethylcyclobutyl)-methyl)morpholine
[46] 1-(2-((1R,3R)-2,2-dimethyl-3-(morpholinomethyl)cyclobutyl)ethyl)-6,7-dihydro-1H-indol-4(5H)-one
[47] 2-(2-((1R,3R)-2,2-dimethyl-3-(morpholinomethyl)cyclobutyl)ethyl)-1,2,3,4-tetrahydroisoquinoline
[48] 1-(((1R,3R)-3-(2-(1H-imidazol-1-yl)ethyl)-2,2-dimethylcyclobutyl)-methyl)piperidine
[49] 1-(((1R,3R)-3-(2-(1H-pyrazol-1-yl)ethyl)-2,2-dimethylcyclobutyl)-methyl)piperidine
[50] 1-(((1R,3R)-3-(2-(1H-1,2,4-triazol-1-yl)ethyl)-2,2-dimethylcyclobutyl)-methyl)piperidine
[51] 1-(2-((1R,3R)-2,2-dimethyl-3-(piperidin-1-ylmethyl)cyclobutyl)ethyl)-4-phenylpiperidine
[52] 4-benzyl-1-(2-((1R,3R)-2,2-dimethyl-3-(piperidin-1-ylmethyl)cyclobutyl)-ethyl)piperidine
[53] 1-(2-((1R,3R)-2,2-dimethyl-3-(piperidin-1-ylmethyl)cyclobutyl)ethyl)-6,7-dihydro-1H-indol-4(5H)-one
[54] 2-(24(1R,3R)-2,2-dimethyl-3-(piperidin-1-ylmethyl)cyclobutyl)ethyl)-1,2,3,4-tetrahydroisoquinoline
[55] 2-((1R,3R)-2,2-Dimethyl-3-((piperidin-1-yl)methyl)cyclobutyl)ethyl pivalate
[56] 2-((1R,3R)-2,2-Dimethyl-3-((piperidin-1-yl)methyl)cyclobutyl)ethyl acetate
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

25. Process for the preparation of a compound of general formula I according to one or more of claims 1 to 24, characterized in that at least one compound of general formula II, 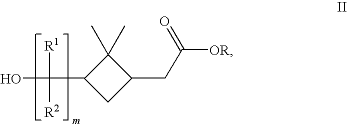
wherein m, R¹ and R² have the meaning according to one or more of claims 1 to 24 and R represents a linear or branched C₁₋₅-alkyl radical or a benzyl radical,
is reacted with methane sulfonylchloride, p-toluene sulfonylchloride, trifluormethane sulfonylchloride, thionyl chloride or tetrabromethane, preferably in a reaction medium, preferably in the presence of at least one base, to yield at least one compound of general formula III, 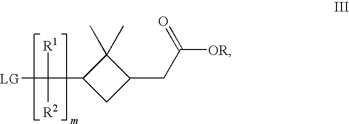
wherein m, R¹ and R² have the meaning given above, R represents a linear or branched C₁₋₅-alkyl radical and LG represents —O—S(═O)₂—CH₃, —O—S(═O)₂-p-toluyl, —O—S(═O)₂—CF₃, Cl or Br, which is optionally purified and/or isolated,
and at least one compound of general formula III is reacted with at least one compound of general formula HNR⁶R⁷, wherein R⁶ and R⁷ have the meaning according to one or more of claims 1 to 24, preferably in a reaction medium, preferably in the presence of at least one base, to yield at least one compound of general formula IV, 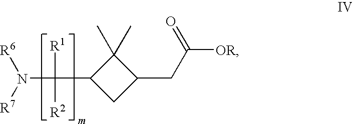
wherein m, R¹, R², R⁶ and R⁷ have the meaning given above and R represents a linear or branched C₁₋₅-alkyl radical, which is optionally purified and/or isolated,
and at least one compound of general formula IV is reacted with at least one reducing agent selected from the group consisting of lithium borohydride, sodium borohydride, lithium aluminium hydride and diborane, preferably with lithium borohydride, preferably in a reaction medium, to yield at least one compound of general formula V, 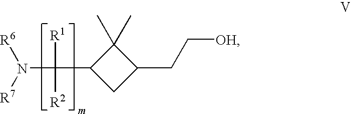
wherein m, R¹, R², R⁶ and R⁷ have the meaning given above, which is optionally purified and/or isolated,
and at least one compound of general formula V is reacted with methane sulfonylchloride, p-toluene sulfonylchloride, trifluormethane sulfonylchloride, thionyl chloride or tetrabromethane,
preferably in a reaction medium, preferably in the presence of at least one base, to yield at least one compound of general formula VI, 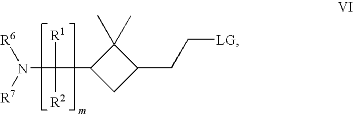
wherein m, R¹, R², R⁶ and R⁷ have the meaning given above and LG represents —O—S(═O)₂—CH₃, —O—S(═O)₂-p-toluyl, —O—S(═O)₂—CF₃, Cl or Br, which is optionally purified and/or isolated,
and at least one compound of general formula VI is reacted with at least one compound of general formula HNR⁹R¹⁰, wherein R⁹ and R¹⁰ have the meaning according to one or more of claims 1 to 24, preferably in a reaction medium, preferably in the presence of at least one base, to yield at least one compound of general formula VII, 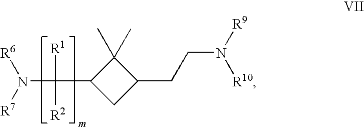
wherein m, R¹, R², R⁶, R⁷, R⁹ and R¹⁰ have the meaning given above, which is optionally purified and/or isolated,
or at least one compound of general formula VI is reacted with at least one compound of general formula M—OR⁸, wherein R⁸ has the meaning according to one or more of claims 1 to 24 and M represents a monovalent kation selected from the group consisting of sodium, magnesium, potassium and lithium, preferably M represents a lithium kation, preferably in a reaction medium, preferably in the presence of at least one base, to yield at least one compound of general formula VIII, 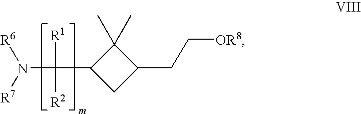
wherein m, R¹, R², R⁶, R⁷ and R⁸ have the meaning given above, which is optionally purified and/or isolated;
and optionally at least one compound of general formula VIII, wherein R⁸ represents hydrogen, is reacted with at least one compound of general formula LG-C(═O)—R¹², wherein R¹² has the meaning according to one or more of claims 1 to 24 and LG represents a leaving group, preferably a leaving group selected from the group consisting of chlorine and bromine, preferably in a reaction medium, preferably in the presence of at least one base,
or with at least one compound of general formula HO—C(═O)—R¹², wherein R¹² has the meaning according to one or more of claims 1 to 24, preferably in a reaction medium, preferably in the presence of at least one base, preferably in the presence of at least one coupling agent, 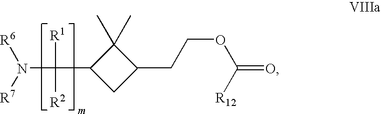
to yield at least one compound of general formula VIIIa,
wherein m, R¹, R², R⁶, R⁷ and R¹² have the meaning given above, which is optionally purified and/or isolated.

26. Process for the preparation of a compound of general formula I according to one or more of claims 1 to 24, characterized in that at least one compound of general formula II, 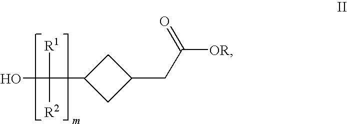
wherein m, R¹ and R² have the meaning according to one or more of claims 1 to 24 and R represents a linear or branched C₁₋₅-alkyl radical, is reacted with at least one compound of general formula R⁵-LG, wherein R⁵ has the meaning according to one or more of claims 1 to 24 and LG represents a leaving group, preferably LG represents a leaving group selected from the group consisting of chlorine, bromine, —O—S(═O)₂—CH₃, —O—S(═O)₂—CF₃ and —O—S(═O)₂-p-toluyl, more preferably LG represents bromine, preferably in a reaction medium, preferably in the presence of at least one base, more preferably in the presence of at least one base selected from the group consisting of butyllithium, sodium ethoxide, potassium tert-butoxide, sodium hydride and potassium hydride, to yield at least one compound of general formula IX, 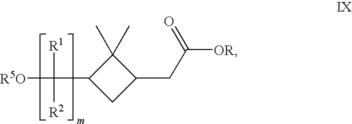
wherein m, R¹, R² and R⁵ have the meaning given above and R represents a linear or branched C₁₋₅-alkyl radical, which is optionally purified and/or isolated,
and at least one compound of general formula IX is reacted with at least one reducing agent selected from the group consisting of lithium borohydride, sodium borohydride, lithium aluminium hydride and diborane, preferably with lithium borohydride, preferably in a reaction medium, to yield at least one compound of general formula X, 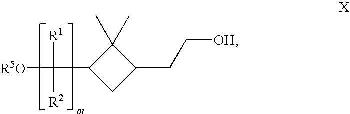
wherein m, R¹, R² and R⁵ have the meaning given above, which is optionally purified and/or isolated,
and optionally at least one compound of general formula X, is reacted with at least one compound of general formula LG-C(═O)—R¹², wherein R¹² has the meaning according to one or more of claims 1 to 24 and LG represents a leaving group, preferably a leaving group selected from the group consisting of chlorine and bromine, preferably in a reaction medium, preferably in the presence of at least one base, or with at least one compound of general formula HO—C(═O)—R¹², wherein R¹² has the meaning according to one or more of claims 1 to 24, preferably in a reaction medium, preferably in the presence of at least one base, preferably in the presence of at least one coupling agent,
to yield at least one compound of general formula Xa, 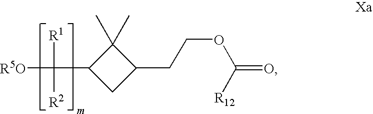
wherein m, R¹, R² and R⁵ have the meaning given above, which is optionally purified and/or isolated,
and at least one compound of general formula X is reacted with methane sulfonylchloride, p-toluene sulfonylchloride, trifluormethane sulfonylchloride, thionyl chloride or tetrabromethane, preferably in a reaction medium, preferably in the presence of at least one base, to yield at least one compound of general formula XI, 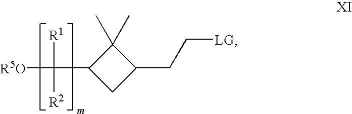
wherein m, R¹, R² and R⁵ have the meaning given above and LG represents —O—S(═O)₂—CH₃, —O—S(═O)₂-p-toluyl, —O—S(═O)₂—CF₃, Cl or Br, which is optionally purified and/or isolated,
and at least one compound of general formula XI is reacted with at least one compound of general formula HNR⁹R¹⁰, wherein R⁹ and R¹⁰ have the meaning according to one or more of claims 1 to 24, preferably in a reaction medium, preferably in the presence of at least one base, to yield at least one compound of general formula XII, 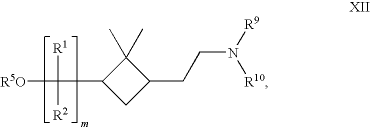
wherein m, R¹, R², R⁵, R⁹ and R¹⁰ have the meaning given above, which is optionally purified and/or isolated.

27. Process for the preparation of a compound of general formula I according to one or more of claims 1 to 24, characterized in that at least one compound of general formula XIII, 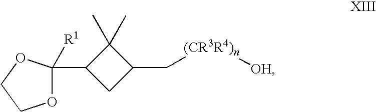
wherein R¹, R³ and R⁴ have the meaning according to one or more of claims 1 to 24, is reacted with methane sulfonylchloride, p-toluene sulfonylchloride, trifluormethane sulfonylchloride, thionyl chloride or tetrabromethane,
preferably in a reaction medium, preferably in the presence of at least one base, to yield at least one compound of general formula XIV, 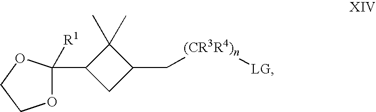
wherein R¹, R³ and R⁴ have have the meaning given above and LG represents —O—S(═O)₂—CH₃, —O—S(═O)₂-p-toluyl, —O—S(═O)₂—CF₃, Cl or Br, which is optionally purified and/or isolated,
and at least one compound of general formula XIV is reacted with at least one compound of general formula HNR⁹R¹⁰, wherein R⁹ and R¹⁰ have the meaning according to one or more of claims 1 to 24, preferably in a reaction medium, preferably in the presence of at least one base, to yield at least one compound of general formula XV, 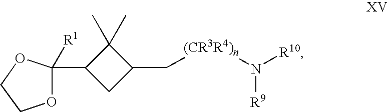
wherein R¹, R³, R⁴, R⁹ and R¹⁰ have the meaning given above, which is optionally purified and/or isolated,
and at least one compound of general formula XV is reacted with at least one reagent selected from the group consisting of hydrochloric acid, pyridinium p-toluenesulfonate, sulfonic acid and trifluoroacetic acid, preferably with pyridinium p-toluenesulfonate, preferably in a reaction medium, to yield at least one compound of general formula XVI, 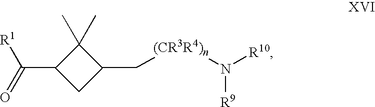
wherein R¹, R³, R⁴, R⁹ and R¹⁰ have the meaning given above, which is optionally purified and/or isolated,
and at least one compound of general formula XVI is reacted with at least one compound of general formula R²—Li, wherein R² has the meaning according to one or more of claims 1 to 24, or R²—Mg—Z, wherein R² has the meaning according to one or more of claims 1 to 24 and Z represents an anion selected from the group consisting of bromide and chloride, preferably in a reaction medium, to yield at least one compound of general formula XVII, 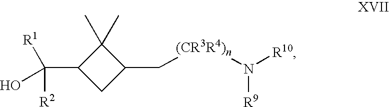
wherein R¹, R², R³, R⁴, R⁹ and R¹⁰ have the meaning given above, which is optionally purified and/or isolated,
and optionally at least one compound of general formula XVII is reacted with at least one compound of general formula R⁵-LG, wherein R⁵ has the meaning according to one or more of claims 1 to 24 and LG represents a leaving group, preferably LG represents a leaving group selected from the group consisting of chlorine, bromine, —O—S(═O)₂—CH₃, —O—S(═O)₂—CF₃ and —O—S(═O)₂-p-toluyl more preferably LG represents bromine, preferably in a reaction medium, preferably in the presence of at least one base, more preferably in the presence of at least one base selected from the group consisting of butyllithium, sodium ethoxide, potassium tert-butoxide, sodium hydride and potassium hydride, to yield at least one compound of general formula XVIII, 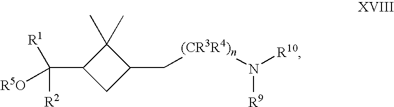
wherein R¹, R², R³, R⁴, R⁵, R⁹ and R¹⁰ have the meaning given above, which is optionally purified and/or isolated.

28. Process for the preparation of a compound of genera formula I according to one or more of claims 1 to 24, characterized in that at least one compound of general formula IXX, 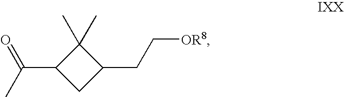
wherein R⁸ has the meaning according to one or more of claims 1 to 24, is reacted with NaOBr, preferably in a reaction medium, more preferably in a reaction medium selected from the group consisting of dioxane, THF and water or a mixture thereof, to yield at least one compound of general formula XX, 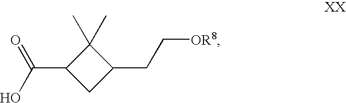
wherein R⁸ has the meaning according to one or more of claims 1 to 24, which is optionally purified and/or isolated,
and at least one compound of general formula XX is reacted with methyl iodide, preferably in a reaction medium, preferably in the presence of at least one base, more preferably in the presence of caesium carbonate, to yield at least one compound of general formula 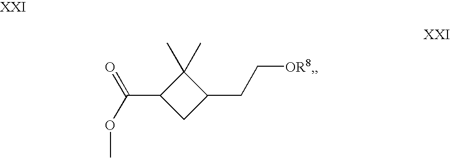
wherein R⁸ has the meaning according to one or more of claims 1 to 24, which is optionally purified and/or isolated,
and at least one compound of general formula XXI is reacted with at least one reducing agent selected from the group consisting of lithium borohydride, sodium borohydride, lithium aluminium hydride and diborane, preferably with lithium borohydride, preferably in a reaction medium, to yield at least one compound of general formula XXII, 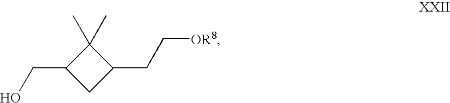
wherein R⁸ has the meaning according to one or more of claims 1 to 24, which is optionally purified and/or isolated.

29. A medicament comprising at least one compound of general formula I according to one or more of claims 1 to 24 and optionally at least one physiologically acceptable auxiliary agent.

30. A medicament according to claim 29 for the treatment and/or prophylaxis of sigma receptor mediated disease or disorder.

31. A medicament according to claim 29 or 30 for the prophylaxis and/or treatment of a disorder or disease related to food intake, preferably for the regulation of appetite, for the maintenance, increase or reduction of body weight, for the prophylaxis and/or treatment of obesity, bulimia, anorexia, cachexia, type II diabetes (non insulin dependent diabetes mellitus), preferably type II diabetes that is caused by obesity; for the prophylaxis and/or treatment of diarrhoea; lipoprotein disorders, preferably selected from the group consisting of hypercholesterolemia (type II hyperlipoproteinemia); hypertriglyceridemia; hypoalphalipoproteinemia and high lipoprotein(a) levels; arthritis; hypertension; arrhythmia; ulcer; tardive dyskinesia; stress; cancer; stroke; ischemic stroke; migraine; epilepsy; anxiety; panic attacks; depression; cognitive disorders; cognitive dysfunction associated with psychiatric diseases; memory disorders; psychosis; schizophrenia; for the prophylaxis and/or treatment of drug addiction and/or withdrawal or for the prophylaxis and/or treatment of cocaine addiction and/or withdrawal.

32. A medicament according to claim 29 or 30 for the prophylaxis and/or treatment of pain, preferably neuropathic pain, allodynia, analgesia, causalgia, central pain, dysesthesia, hyperesthesia, hyperalgesia, hypoalgesia, hypoesthesia, or neuralgia, more preferably neuropathic pain, hyperalgesia or allodynia.

33. Use of at least one substituted dimethylcyclobutyl compound of general formula I, 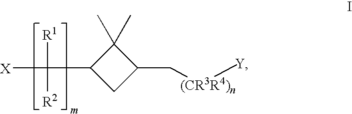
wherein
m is 1, 2, 3 or 4;
n is 0, 1, 2 or 3;
X represents a —OR⁵ moiety or a —NR⁶R⁷ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
Y represents a —OR⁸ moiety; a —NR⁹R¹⁰ moiety; a —C(═O)—OR¹¹ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R¹ represents a hydrogen atom or a linear or branched, saturated or
unsaturated, unsubstituted or at least mono-substituted aliphatic radical;
R² represents a hydrogen atom;
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, saturated or unsaturated, unsubstituted or at least mono-substituted aliphatic radical;
R⁵ and R⁸, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system
or a —C(═O)—R¹² moiety;
R⁶, R⁷, R⁹ and R¹⁰, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
and
R¹¹ and R¹², independently of one other, represents a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof,
for the manufacture of a medicament for treatment and/or prophylaxis of sigma receptor mediated disease or disorder.

34. Use of at least one substituted dimethylcyclobutyl compound of general formula I, 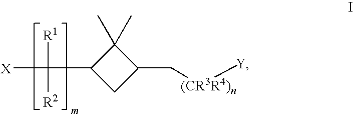
wherein
m is 1, 2, 3 or 4;
n is 0, 1, 2 or 3;
X represents a —OR⁵ moiety or a —NR⁶R⁷ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
Y represents a —OR⁸ moiety; a —NR⁹R¹⁰ moiety; a —C(═O)—OR¹¹ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R¹ represents a hydrogen atom or a linear or branched, saturated or
unsaturated, unsubstituted or at least mono-substituted aliphatic radical;
R² represents a hydrogen atom;
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, saturated or unsaturated, unsubstituted or at least mono-substituted aliphatic radical;
R⁵ and R⁸, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system
or a —C(═O)—R¹² moiety;
R⁶, R⁷, R⁹ and R¹⁰, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
and
R¹¹ and R¹², independently of one other, represents a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system; optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof,
for the manufacture of a medicament for the prophylaxis and/or treatment of a disorder or disease related to food intake, preferably for the regulation of appetite, for the maintenance, increase or reduction of body weight, for the prophylaxis and/or treatment of obesity, bulimia, anorexia, cachexia, type II diabetes (non insulin dependent diabetes mellitus), preferably type II diabetes that is caused by obesity; for the prophylaxis and/or treatment of diarrhoea; lipoprotein disorders, preferably selected from the group consisting of hypercholesterolemia (type II hyperlipoproteinemia); hypertriglyceridemia; hypoalphalipoproteinemia and high lipoprotein(a) levels; arthritis; hypertension; arrhythmia; ulcer; tardive dyskinesia; stress; cancer; stroke; ischemic stroke; migraine; epilepsy; anxiety; panic attacks; depression; cognitive disorders; cognitive dysfunction associated with psychiatric diseases; memory disorders; psychosis; schizophrenia; for the prophylaxis and/or treatment of drug addiction and/or withdrawal or for the prophylaxis and/or treatment of cocaine addiction and/or withdrawal.

35. Use of at least one substituted dimethylcyclobutyl compound of general formula I, 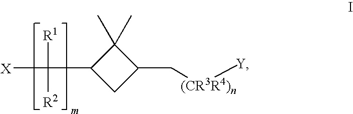
wherein
m is 1, 2, 3 or 4;
n is 0, 1, 2 or 3;
X represents a —OR⁵ moiety or a —NR⁶R⁷ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
Y represents a —OR⁸ moiety; a —NR⁹R¹⁰ moiety; a —C(═O)—OR¹¹ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R¹ represents a hydrogen atom or a linear or branched, saturated or
unsaturated, unsubstituted or at least mono-substituted aliphatic radical;
R² represents a hydrogen atom;
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, saturated or unsaturated, unsubstituted or at least mono-substituted aliphatic radical;
R⁵ and R⁸, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system
or a —C(═O)—R¹² moiety;
R⁶, R⁷, R⁹ and R¹⁰, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
and
R¹¹ and R¹², independently of one other, represents a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof,
for the manufacture of a medicament for the prophylaxis and/or treatment of pain, preferably neuropathic pain, allodynia, analgesia, causalgia, central pain, dysesthesia, hyperesthesia, hyperalgesia, hypoalgesia, hypoesthesia, or neuralgia, more preferably neuropathic pain, hyperalgesia or allodynia.

36. Use of at least one substituted dimethylcyclobutyl compound of general formula I, 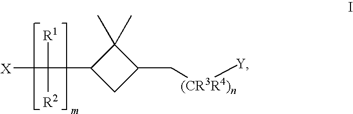
wherein
m is 1, 2, 3 or 4;
n is 0, 1, 2 or 3;
X represents a —OR⁵ moiety or a —NR⁶R⁷ moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
Y represents a —R⁸ moiety; a —NR⁹R¹⁰ moiety; a —C(═O)—OR¹¹
moiety;
a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R¹ represents a hydrogen atom or a linear or branched, saturated or
unsaturated, unsubstituted or at least mono-substituted aliphatic radical;
R² represents a hydrogen atom;
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
R³ and R⁴, independently of one another, each represent a hydrogen atom or a linear or branched, saturated or unsaturated, unsubstituted or at least mono-substituted aliphatic radical;
R⁵ and R⁸, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system
or a —C(═O)—R¹² moiety;
R⁶, R⁷, R⁹ and R¹⁰, independently of one another, each represent a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
or R⁶ and R⁷, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
or R⁹ and R¹⁰, together with the bridging nitrogen atom form an unsubstituted or at least mono-substituted, saturated, unsaturated or aromatic heterocyclic ring which may contain one or more additional heteroatom(s) as ring member(s) and which may be condensed with an unsubstituted or at least mono-substituted saturated, unsaturated or aromatic mono- or bicyclic ring system;
and
R¹¹ and R¹², independently of one other, represents a hydrogen atom or a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched, unsubstituted or at least-mono substituted alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched, unsubstituted or at least-mono substituted alkylene, alkenylene or alkinylene group and which may be condensed with an unsubstituted or at least mono-substituted saturated or unsaturated, but not aromatic, mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof,
as pharmacological tool or as anxiolytic or as immunosuppressant.
